SEQUENCE LISTING (242 sequences)

SEQ ID NO: 1 - protein, 13 aa, Streptococcus pyogenes
AHIVMVDAYKPTK

SEQ ID NO: 2 - protein, 13 aa, Streptococcus dysgalactiae
DPIVMIDNDKPIT

SEQ ID NO: 3 - protein, 12 aa, Streptococcus pneumoniae
KLGDIEFIKVNK

SEQ ID NO: 4 - protein, 22 aa, Streptococcus phocae
LVTGTAHIVMVDNYKPIVETGD

SEQ ID NO: 5 - protein, 15 aa, Enterococcus faecalis
NTIVMVDKLKEVPPT

SEQ ID NO: 6 - protein, 20 aa, Ruminococcus sp.
  source 1..20: note = Ruminococcus sp. AF26-25AA
SENGNPLIVMVDDTTKVKIS

SEQ ID NO: 7 - protein, 17 aa, Ruminococcus sp.
  source 1..17: note = Ruminococcus sp. AF25-19
GTPIVIMVDEAKPSLPD

SEQ ID NO: 8 - protein, 17 aa, Ruminococcus sp.
  source 1..17: note = Ruminococcus sp. Marseille-P6503
GNPLIVMIDEAEQKEIP

SEQ ID NO: 9 - protein, 17 aa, Ruminococcus sp.
AGGIIVMKDNTTKVSIS

SEQ ID NO: 10 - protein, 17 aa, Ruminococcus flavefaciens
GNPIVTMIDDATLVKIS

SEQ ID NO: 11 - protein, 17 aa, Ruminococcus sp.
GNSTITMVDDTTKVHIT

SEQ ID NO: 12 - protein, 17 aa, Ruminococcus sp.
  source 1..17: note = Ruminococcus sp. AM43-6
GTPLIVMVDDTTKVEIS

SEQ ID NO: 13 - protein, 15 aa, synthetic construct
  REGION 1..15: note = Rumtrunk D9N
  REGION 1..15: note = MISC_FEATURE - Rumtrunk D9N
GNPLIVMVNDTTKVK

SEQ ID NO: 14 - protein, 15 aa, Ruminococcus sp.
GNPLIVMVDDTTKVK

SEQ ID NO: 15 - protein, 20 aa, Bacillus cereus
NEKVTGQFEIVKVDANDKTK

SEQ ID NO: 16 - protein, 19 aa, Bacillus cereus
SKSLGQFEIVKVDAQDKTK

SEQ ID NO: 17 - protein, 16 aa, Bacillus cereus
LGQFEIVKVDSQDKTK

SEQ ID NO: 18 - protein, 17 aa, Bacillus cereus
VTGQFEIVKVDAEDKTR

SEQ ID NO: 19 - protein, 19 aa, Bacillus cereus
EKVMGQFEIMKVDANDKTK

SEQ ID NO: 20 - protein, 23 aa, Clostridium perfringens
DTKQVVKHEDKNDKAQTLVVEKP

SEQ ID NO: 21 - protein, 116 aa, Streptococcus pyogenes
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 22 - protein, 104 aa, Streptococcus dysgalactiae
IDTMSGLSGETGQSGNTTIEEDSTTHVKFSKRDSNGKELAGAMIELRNLSGQTIQSWVSDGTVKDFYLMPGTYQFVETAAPEGYELAAPITFTIDEKGQIWVDS

SEQ ID NO: 23 - protein, 123 aa, Streptococcus pneumoniae
SSGLVPRGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 24 - protein, 129 aa, Actinomyces viscosus
GSLSKYGKVILTKTGTDDLADKTKYNGAQFQVYECTKTASGATLRDSDPSTQTVDPLTIGGEKTFTTAGQGTVEINYLRANDYVNGAKKDQLTDEDYYCLVETKAPEGYNLQADPLPFRVLAEKAEKKA

SEQ ID NO: 25 - protein, 102 aa, Streptococcus pneumoniae
GSTTKVKLIKVDQDHNRLEGVGFKLVSVARDVSAAAVPLIGEYRYSSSGQVGRTLYTDKNGEIFVTNLPLGNYRFKEVEPLAGYAVTTLDTDVQLVDHQLVT

SEQ ID NO: 26 - protein, 94 aa, Streptococcus pneumoniae
PRGNVDFMKVDGRTNTSLQGAMFKVMKEESGHYTPVLQNGKEVVVTSGKDGRFRVEGLEYGTYYLWELQAPTGYVQLTSPVSFTIGKDTRKELV

SEQ ID NO: 27 - protein, 123 aa, Corynebacterium diphtheriae
VVTYHGKLKVVKKDGKEAGKVLKGAEFELYQCTSAAVLGKGPLTVDGVKKWTTGDDGTFTIDGLHVTDFEDGKEAAPATKKFCLKETKAPAGYALPDPNVTEIEFTRAKISEKDKFEGDDEVT

SEQ ID NO: 28 - protein, 134 aa, Lactobacillus rhamnosus
STNDTTTQNVVLTKYGFDKDVTAIDRATDQIWTGDGAKPLQGVDFTIYNVTANYWASPKDYKGSFDSAPVAATGTTNDKGQLTQALPIQSKDASGKTRAAVYLFHETNPRAGYNTSADFWLTLPAKAAADGNVY

SEQ ID NO: 29 - protein, 114 aa, Lactobacillus rhamnosus
TTYERTFVKKDAETKEVLEGAGFKISNSDGKFLKLTDKDGQSVSIGEGFIDVLANNYRLTWVAESDATVFTSDKSGKFGLNGFADNTTTYTAVETNVPDGYDAAANTDFKADNS

SEQ ID NO: 30 - protein, 107 aa, Streptococcus agalactiae
GQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNP

SEQ ID NO: 31 - protein, 91 aa, Streptococcus intermedius
EQDVVFSKVNVAGEEIAGAKIQLKDAQGQVVHSWTSKAGQSETVKLKAGTYTFHEASAPTGYLAVTDITFEVDVQGKVTVKDANGNGVKAD

SEQ ID NO: 32 - protein, 104 aa, Streptococcus pneumoniae
KLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQ

SEQ ID NO: 33 - protein, 130 aa, Corynebacterium diphtheriae
GSERKGSLTLHKKKGAESEKRATGKEMDDVAGEPLNGVTFKITKLNFDLQNGDWAKFPKTAADAKGHETSTTKEVETSGNGTAVFDNLDLGIYLVEETKAPDGIVTGAPFIVSIPMVNEASDAWNYNVVA

SEQ ID NO: 34 - protein, 149 aa, Clostridium perfringens
NLPEVKDGTLRTTVIADGVNGSSEKEALVSFENSKDGVDVKDTINYEGLVANQNYTLTGTLMHVKADGSLEEIATKTTNVTAGENGNGTWGLDFGNQKLQVGEKYVVFENAESVENLIDTDKDYNLDTKQVVKHEDKNDKAQTLVVEKP

SEQ ID NO: 35 - DNA, 39 bp, Streptococcus pyogenes
  source 1..39: mol_type = other DNA
gctcacatcgtgatggtggacgcttacaagcccaccaag

SEQ ID NO: 36 - DNA, 39 bp, Streptococcus dysgalactiae
  source 1..39: mol_type = other DNA
gaccccatcgtgatgatcgacaacgacaagcccatcacc

SEQ ID NO: 37 - DNA, 36 bp, Streptococcus pneumoniae
  source 1..36: mol_type = other DNA
aaactgggtgatattgaatttattaaagttaataaa

SEQ ID NO: 38 - DNA, 66 bp, Streptococcus phocae
  source 1..66: mol_type = other DNA
ctggttaccggcaccgcacatattgttatggttgataactataagccgatcgtggaaaccggtgat

SEQ ID NO: 39 - DNA, 60 bp, Enterococcus faecalis
  source 1..60: mol_type = other DNA
accgaagttagcggtaataccattgtgatggtggataaactgaaagaagttccgcctacc

SEQ ID NO: 40 - DNA, 60 bp, Ruminococcus sp.
  source 1..60: mol_type = other DNA; note = Ruminococcus sp. AF26-25AA
agcgaaaacggcaacccgctgattgtgatggtggatgataccaccaaagtgaaaattagc

SEQ ID NO: 41 - DNA, 51 bp, Ruminococcus sp.
  source 1..51: mol_type = other DNA; note = Ruminococcus sp. AF25-19
ggcaccccgattgtgattatggtggatgaagcgaaaccgagcctgccggat

SEQ ID NO: 42 - DNA, 51 bp, Ruminococcus sp.
  source 1..51: mol_type = other DNA; note = Ruminococcus sp. Marseille-P6503
ggcaacccgctgattgtgatgattgatgaagcggaacagaaagaaattccg

SEQ ID NO: 43 - DNA, 51 bp, Ruminococcus sp.
  source 1..51: mol_type = other DNA
gcgggcggcattattgtgatgaaagataacaccaccaaagtgagcattagc

SEQ ID NO: 44 - DNA, 48 bp, Ruminococcus flavefaciens
  source 1..48: mol_type = other DNA
ggcaacccgattgtgaccatgattgatgatgcgaccctggtgaaaatt

SEQ ID NO: 45 - DNA, 51 bp, Ruminococcus sp.
  source 1..51: mol_type = other DNA
ggcaacagcaccattaccatggtggatgataccaccaaagtgcatattacc

SEQ ID NO: 46 - DNA, 51 bp, Ruminococcus sp.
  source 1..51: mol_type = other DNA; note = Ruminococcus sp. AM43-6
ggcaccccgctgattgtgatggtggatgataccaccaaagtggaaattagc

SEQ ID NO: 47 - DNA, 45 bp, synthetic construct
  misc_feature 1..45: note = Rumtrunk D9N DNA
  misc_feature 1..45: note = Rumtrunk D9N
  source 1..45: mol_type = other DNA
ggtaatccgctgattgtgatggtgaatgataccaccaaagtgaaa

SEQ ID NO: 48 - DNA, 45 bp, Ruminococcus sp.
  source 1..45: mol_type = other DNA
ggcaacccgctgattgtgatggtggatgataccaccaaagtgaaa

SEQ ID NO: 49 - DNA, 45 bp, Bacillus cereus
  source 1..45: mol_type = other DNA
ggtcagttcgaaattgttaaagttgatgcaaacgataaaactaaa

SEQ ID NO: 50 - DNA, 57 bp, Bacillus cereus
  source 1..57: mol_type = other DNA
agcaaaagcctgggccagtttgaaattgtgaaagtggatgcgcaggataaaaccaaa

SEQ ID NO: 51 - DNA, 48 bp, Bacillus cereus
  source 1..48: mol_type = other DNA
ctgggccagtttgaaattgttaaagttgatagccaggataaaaccaaa

SEQ ID NO: 52 - DNA, 51 bp, Bacillus cereus
  source 1..51: mol_type = other DNA
gttaccggtcagtttgaaatcgttaaagttgatgccgaagataagacccgt

SEQ ID NO: 53 - DNA, 57 bp, Bacillus cereus
  source 1..57: mol_type = other DNA; note = Bacillus cereus VD022
gaaaaagtgatgggccagttcgaaatcatgaaagttgatgccaacgacaagaccaaa

SEQ ID NO: 54 - DNA, 69 bp, Clostridium perfringens
  source 1..69: mol_type = other DNA
gacaccaagcaggtggtgaagcacgaggacaagaacgacaaggcccagaccctggtggtggagaagccc

SEQ ID NO: 55 - DNA, 348 bp, Streptococcus pyogenes
  source 1..348: mol_type = other DNA
ggtgcaatggttgataccctgagcggtctgagcagcgaacagggtcagagcggtgatatgaccattgaagaagatagcgcaacccacatcaaattcagcaaacgtgatgaagatggtaaagaactggcaggcgcaacaatggaactgcgtgatagcagcggtaaaaccattagcacctggattagtgatggtcaggtgaaagatttttatctgtaccctggcaaatacacctttgttgaaaccgcagcaccggatggttatgaagttgcaaccgcaattacctttaccgttaatgaacagggccaggttaccgtgaatggtaaagcaaccaaaggtgatgcacatatt

SEQ ID NO: 56 - DNA, 318 bp, Streptococcus dysgalactiae
  source 1..318: mol_type = other DNA
atgggtattgataccatgagcggtctgagcggtgaaaccggtcagagcggtaataccaccattgaagaggatagcaccacacatgtgaaattcagcaaacgcgatgcaaacggcaaagaactggcaggcgcaatgattgaactgcgtaatctgagtggtcagaccattcagagctgggttagtgatggcaccgttaaagatttttatctgatgcctggcacctatcagtttgttgaaaccgcagcaccggaaggttatgagctggcagcaccgattacctttaccattgatgaaaaaggtcagatttgggttgatagc

SEQ ID NO: 57 - DNA, 369 bp, Streptococcus pneumoniae
  source 1..369: mol_type = other DNA
agcagcggcctggtgccgcgcggcagccatatgaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcaggatattccggctacatatgaatttaccaacggtaaacattatatcaccaatgaaccgataccgccgaaa

SEQ ID NO: 58 - DNA, 387 bp, Actinomyces viscosus
  source 1..387: mol_type = other DNA
ggtagcctgagcaaatatggtaaagtgattctgaccaaaaccggcaccgatgatctggcagataaaaccaaatataacggtgcacagtttcaggtgtatgaatgtaccaaaacagcaagcggtgcaaccctgcgtgatagcgatccgagcacacagaccgttgatccgctgaccattggtggtgaaaaaacctttaccaccgcaggtcagggcaccgttgaaattaattatctgcgtgccaatgattatgtgaacggtgcaaaaaaagatcagctgaccgatgaagattattactgtctggttgaaaccaaagcaccggaaggttataatctgcaggcagatccgctgccgtttcgtgttctggccgaaaaagcagaaaaaaaagcc

SEQ ID NO: 59 - DNA, 306 bp, Streptococcus pneumoniae
  source 1..306: mol_type = other DNA
ggtagcaccaccaaagtgaaactgattaaagttgatcaggatcacaatcgtctggaaggtgttggttttaaactggttagcgttgcacgtgatgttagcgcagcagcagttccgctgattggtgaatatcgttatagcagcagcggtcaggttggtcgtaccctgtataccgataaaaatggcgaaattttcgttaccaatctgccgctgggtaactatcgttttaaagaagttgaaccgctggcaggttatgcagttaccacactggataccgatgttcagctggttgatcatcagctggtgacc

SEQ ID NO: 60 - DNA, 282 bp, Streptococcus pneumoniae
  source 1..282: mol_type = other DNA
ccgcgtggtaatgttgattttatgaaagttgatggtcgcaccaataccagcctgcagggtgcaatgtttaaagtgatgaaagaagaaagcggtcactatacaccggtgctgcagaatggtaaagaagttgttgttaccagcggtaaagatggtcgttttcgtgttgaaggtctggaatatggcacctattatctgtgggaactgcaggcaccgaccggttatgttcagctgaccagtccggttagttttaccattggcaaagatacccgtaaagaactggtg

SEQ ID NO: 61 - DNA, 369 bp, Corynebacterium diphtheriae
  source 1..369: mol_type = other DNA
gttgttacctatcatggtaaactgaaagtggtgaaaaaagacggtaaagaggcaggcaaagttctgaaaggtgcagaatttgaactgtatcagtgtaccagcgcagcagttttaggtaaaggtccgctgaccgttgatggtgtgaaaaaatggaccaccggtgatgatggcacctttaccattgatggtctgcatgttaccgattttgaagatggtaaagaagccgcaccggcaaccaaaaaattctgtctgaaagaaaccaaagcaccggcaggttatgcactgcctgatccgaatgtgaccgaaattgaatttacccgtgcaaaaatcagcgagaaagataaatttgaaggcgacgatgaagtgacc

SEQ ID NO: 62 - DNA, 402 bp, Lactobacillus rhamnosus
  source 1..402: mol_type = other DNA
agcaccaatgataccaccacacagaatgttgttctgaccaaatatggcttcgataaagatgttaccgcaattgatcgtgcaaccgatcagatttggaccggtgatggtgcaaaaccgctgcagggtgttgattttaccatttataacgtgaccgccaattattgggcaagcccgaaagattataaaggcagctttgatagcgcaccggttgcagccaccggtacaacaaatgataaaggccagctgacccaggcactgccgattcagagcaaagatgcaagcggtaaaacccgtgcagcagtttacctgtttcacgaaaccaatccgcgtgcaggttataataccagcgcagatttttggctgaccctgcctgcaaaagcagcagcagatggtaatgtttat

SEQ ID NO: 63 - DNA, 342 bp, Lactobacillus rhamnosus
  source 1..342: mol_type = other DNA
accacctatgaacgtacctttgttaaaaaagacgccgaaaccaaagaagttctggaaggcgcaggctttaaaatcagcaatagtgatggcaaattcctgaaactgaccgataaagatggtcagagcgttagcattggtgaaggttttattgatgttctggccaataactatcgtctgacctgggttgcagaaagtgatgcaaccgtttttaccagcgataaaagcggcaaatttggtctgaatggttttgcagataataccaccacctataccgcagttgaaaccaatgttccggatggttatgatgcagcagcaaacaccgatttcaaagccgataatagc

SEQ ID NO: 64 - DNA, 321 bp, Streptococcus agalactiae
  source 1..321: mol_type = other DNA
ggtcagattaccatcaaaaaaatcgatggtagcaccaaagcaagcctgcagggtgcaatttttgttctgaaaaatgcaaccggtcagttcctgaattttaacgataccaataatgttgaatggggcaccgaagcaaatgccaccgaatataccaccggtgcagatggtattattaccattaccggtctgaaagaaggcacctattacctggttgaaaaaaaagcaccgctgggttataatctgctggataattcacagaaagtgattttaggtgatggtgcaaccgataccaccaatagcgataacctgctggttaatccg

SEQ ID NO: 65 - DNA, 273 bp, Streptococcus intermedius
  source 1..273: mol_type = other DNA
gaacaggatgttgtgtttagcaaagttaatgttgccggtgaagaaattgcgggtgcaaaaatccagctgaaagatgcacagggtcaagttgttcatagctggaccagcaaagcaggtcagagcgaaaccgttaaactgaaagcaggcacctatacctttcatgaagcaagcgcaccgaccggttatctggcagttaccgatattacctttgaagttgatgttcagggtaaagtgaccgttaaagatgcaaatggtaatggtgtgaaagccgac

SEQ ID NO: 66 - DNA, 312 bp, Streptococcus pneumoniae
  source 1..312: mol_type = other DNA
aaactgggtgatattgagttcatcaaagtgaacaaaaacgataaaaaaccgctgcgtggtgcagtttttagcctgcagaaacagcatccggattacccggatatttatggtgcaattgatcagaatggcacctatcagaatgttcgtaccggtgaagatggtaaactgacctttaaaaacctgagcgacggtaaatatcgcctgtttgaaaatagcgaaccggcaggttataaaccggttcagaataaaccgattgtggcctttcagattgttaatggtgaagttcgtgatgtgaccagcattgttccgcag

SEQ ID NO: 67 - DNA, 390 bp, Corynebacterium diphtheriae
  source 1..390: mol_type = other DNA
ggtagcgaacgtaaaggtagtctgaccctgcataaaaagaaaggtgcagaaagcgaaaaacgtgcaaccggtaaagaaatggatgatgttgccggtgaaccgctgaatggtgttacctttaaaatcaccaaactgaacttcgatctgcagaatggtgattgggcaaaatttccgaaaaccgcagcagatgcaaaaggtcatgaaaccagcaccaccaaagaagtggaaaccagcggtaatggcaccgcagtttttgataatctggatctgggtatttacctggtggaagaaaccaaagcaccggatggtattgttacaggtgcaccgtttattgttagcattccgatggttaatgaagcaagtgatgcctggaattataacgttgttgca

SEQ ID NO: 68 - DNA, 447 bp, Clostridium perfringens
  source 1..447: mol_type = other DNA
aacctgcccgaggtgaaggacggcaccctgaggaccaccgtgatcgccgacggcgtgaacggcagcagcgagaaggaggccctggtgagcttcgagaacagcaaggacggcgtggacgtgaaggacaccatcaactacgagggcctggtggccaaccagaactacaccctgaccggcaccctgatgcacgtgaaggccgacggcagcctggaggagatcgccaccaagaccaccaacgtgaccgccggcgagaacggcaacggcacctggggcctggacttcggcaaccagaagctgcaggtgggcgagaagtacgtggtgttcgagaacgccgagagcgtggagaacctgatcgacaccgacaaggactacaacctggacaccaagcaggtggtgaagcacgaggacaagaacgacaaggcccagaccctggtggtggagaagccc

SEQ ID NO: 69 - protein, 170 aa, Homo sapiens
RMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 70 - protein, 154 aa, Homo sapiens
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 71 - protein, 122 aa, Homo sapiens
PANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCS

SEQ ID NO: 72 - protein, 205 aa, Homo sapiens
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 73 - protein, 282 aa, Thermotoga maritima
MGARASGSKSGSGSDSGSKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQ

SEQ ID NO: 74 - DNA, 510 bp, Homo sapiens
  source 1..510: mol_type = other DNA
agaatgctgaaggccctgaacgaccagctgaacagagagctgtactccgcctacctgtacttcgctatggccgcctacttcgaggacctgggccttgagggattcgccaactggatgaaggctcaggccgaggaagagatcggccacgctctgagattctacaactacatctacgacagaaacggccgcgtggaactggacgagatccccaagcctcctaaagagtgggagagccctctgaaggctttcgaggctgcttacgagcacgagaagttcatcagcaagagcatctacgagctggccgctctggccgaagaggaaaaggactactctaccagagccttcctggaatggttcatcaacgaacaggtggaagaggaagccagcgtcaagaagatcctggacaagctgaagttcgccaaggacagccctcagatcctgttcatgctggacaaagagctgagcgccagggctcctaaactgcctggactgcttatgcaaggcggcgaa

SEQ ID NO: 75 - DNA, 462 bp, Homo sapiens
  source 1..462: mol_type = other DNA
atgcagatctacgagggcaagctgaccgccgagggcctgaggttcggcatcgtggccagcaggttcaaccacgccctggtggacaggctggtggagggcgccatcgactgcatcgtgaggcacggcggcagggaggaggacatcaccctggtgagggtgcccggcagctgggagatccccgtggccgccggcgagctggccaggaaggaggacatcgacgccgtgatcgccatcggcgtgctgatcaggggcgccaccccccacttcgactacatcgccagcgaggtgagcaagggcctggccaacctgagcctggagctgaggaagcccatcaccttcggcgtgatcaccgccgacaccctggagcaggccatcgagagggccggcaccaagcacggcaacaagggctgggaggccgccctgagcgccatcgagatggccaacctgttcaagagcctgagg

SEQ ID NO: 76 - DNA, 366 bp, Homo sapiens
  source 1..366: mol_type = other DNA
cccgccaacagcaccgtgctgagcttctgcgccttcgccgtggaccccgccaaggcctacaaggactacctggccagcggcggccagcccatcaccaactgcgtgaagatgctgtgcacccacaccggcaccggccaggccatcaccgtgacccccgaggccaacatggaccaggagagcttcggcggcgccagctgctgcctgtactgcaggtgccacatcgaccaccccaaccccaagggcttctgcgacctgaagggcaagtacgtgcagatccccaccacctgcgccaacgaccccgtgggcttcaccctgaggaacaccgtgtgcaccgtgtgcggcatgtggaagggctacggctgcagc

SEQ ID NO: 77 - DNA, 615 bp, Homo sapiens
  source 1..615: mol_type = other DNA
atgaagatggaggagctgttcaagaagcacaagatcgtggccgtgctgagggccaacagcgtggaggaggccaagaagaaggccctggccgtgttcctgggcggcgtgcacctgatcgagatcaccttcaccgtgcccgacgccgacaccgtgatcaaggagctgagcttcctgaaggagatgggcgccatcatcggcgccggcaccgtgaccagcgtggagcaggccaggaaggccgtggagagcggcgccgagttcatcgtgagcccccacctggacgaggagatcagccagttcgccaaggagaagggcgtgttctacatgcccggcgtgatgacccccaccgagctggtgaaggccatgaagctgggccacaccatcctgaagctgttccccggcgaggtggtgggcccccagttcgtgaaggccatgaagggccccttccccaacgtgaagttcgtgcccaccggcggcgtgaacctggacaacgtgtgcgagtggttcaaggccggcgtgctggccgtgggcgtgggcagcgccctggtgaagggcacccccgtggaggtggccgagaaggccaaggccttcgtggagaagatcaggggctgcaccgag

SEQ ID NO: 78 - DNA, 846 bp, Thermotoga maritima
  source 1..846: mol_type = other DNA
atgggcgccagggccagcggcagcaagagcggcagcggcagcgacagcggcagcaagatggaggagctgttcaagaagcacaagatcgtggccgtgctgagggccaacagcgtggaggaggccaagaagaaggccctggccgtgttcctgggcggcgtgcacctgatcgagatcaccttcaccgtgcccgacgccgacaccgtgatcaaggagctgagcttcctgaaggagatgggcgccatcatcggcgccggcaccgtgaccagcgtggagcagtgcaggaaggccgtggagagcggcgccgagttcatcgtgagcccccacctggacgaggagatcagccagttctgcaaggagaagggcgtgttctacatgcccggcgtgatgacccccaccgagctggtgaaggccatgaagctgggccacaccatcctgaagctgttccccggcgaggtggtgggcccccagttcgtgaaggccatgaagggccccttccccaacgtgaagttcgtgcccaccggcggcgtgaacctggacaacgtgtgcgagtggttcaaggccggcgtgctggccgtgggcgtgggcagcgccctggtgaagggcacccccgtggaggtggccgagaaggccaaggccttcgtggagaagatcaggggctgcaccgagcagaagctgatcagcgaggaggacctgcagagcaggcccgagcccaccgccccccccgaggagagcttcaggagcggcgtggagaccaccacccccccccagaagcaggagcccatcgacaaggagctgtaccccctgaccagcctgaggagcctgttcggcaacgaccccagcagccag

SEQ ID NO: 79 - protein, 19 aa, Homo sapiens
MTRLTVLALLAGLLASSRA

SEQ ID NO: 80 - protein, 18 aa, Homo sapiens
MKWVTFISLLFLFSSAYS

SEQ ID NO: 81 - protein, 19 aa, Homo sapiens
MKWVTFISLLFLFSSSSRA

SEQ ID NO: 82 - protein, 20 aa, Cricetulus griseus
MGSAALLLWVLLLWVPGSNG

SEQ ID NO: 83 - protein, 20 aa, Cricetulus griseus
MGSAALLLWVLLLWVPSSRA

SEQ ID NO: 84 - protein, 20 aa, Homo sapiens
MEAPAQLLFLLLLWLPSSRA

SEQ ID NO: 85 - protein, 154 aa, synthetic construct
  REGION 1..154: note = pVax1_ SpyTag-AP205 (amino acid)
  REGION 1..154: note = MISC_FEATURE - pVax1_ SpyTag-AP205
AHIVMVDAYKPTKGSGTAGGGSGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 86 - protein, 198 aa, synthetic construct
  REGION 1..198: note = pVAX1_ HBc-SpyTag (amino acid)
  REGION 1..198: note = MISC_FEATURE - pVAX1_ HBc-SpyTag
MDIDPYKEFGASVELLSFLPSDFFPSIRDLLDTASALYREALESPEHVSPHHTALRQAILCWGELMNLATWVGSNLEDPASRELVVSYVNVNMGLKLRQILWFHISCLTFGRETVLEYLVSFGVWIRTPTAYRPPNAPILSTLPETTVVGGGGGSPGGGTPSPGGGGSQSPGGGGSQSGESQCGSAHIVMVDAYKPTK

SEQ ID NO: 87 - protein, 360 aa, synthetic construct
  REGION 1..360: note = pVAX1-SPYCEGFP (amino acid)
  REGION 1..360: note = MISC_FEATURE - pVAX1-SPYCEGFP
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 88 - DNA, 465 bp, synthetic construct
  misc_feature 1..465: note = pVax1_ SpyTag-AP205 (DNA)
  misc_feature 1..465: note = pVax1_ SpyTag-AP205
  source 1..465: mol_type = other DNA
gctcacatcgtgatggtggacgcctacaagcccacaaaaggctctggaacagctggcggcggatctggctctgccaacaaacctatgcagcccatcaccagcaccgccaacaagatcgtttggagcgaccccaccagactgagcaccacattcagcgctagcctgctgagacagcgcgtgaaagtgggaatcgccgagctgaacaatgtgtccggccagtacgtgtccgtgtacaagaggcctgctcctaagcctgagggctgtgccgatgcctgtgtgatcatgcccaacgagaaccagagcatcagaaccgtgatcagcggcagcgccgagaacctggctacactgaaggctgagtgggagacacacaagagaaacgtggacaccctgttcgcctctggcaacgctggactgggcttcctggatcctacagccgctatcgtgtctagcgacaccacagcctga

SEQ ID NO: 89 - DNA, 597 bp, synthetic construct
  misc_feature 1..597: note = pVAX1_ HBc-SpyTag (DNA)
  misc_feature 1..597: note = pVAX1_ HBc-SpyTag
  source 1..597: mol_type = other DNA
atggacatcgacccctacaaagaatttggcgccagcgtggaactgctgagcttcctgcctagcgacttcttcccttccatccgggacctgctggatacagccagcgcactgtatagagaggccctggaatctcccgagcacgtgtcacctcaccacacagctctgagacaggccatcctgtgttggggcgagctgatgaacctggccacatgggtcggaagcaacctggaagatcccgccagcagagaactggtggtgtcctacgtgaacgtgaacatgggcctgaagctgagacagatcctgtggttccacatcagctgcctgaccttcggcagagaaaccgtgctggaatacctggtgtccttcggcgtgtggatcagaacccctaccgcctacagacctcctaacgctcccatcctgagcaccctgcctgagacaacagttgttggcggaggcggaggatctcctggcggaggaacaccttctccaggtggtggtggatctcaaagtcctggtggtggcggttctcagagcggcgaatctcagtgtggctctgcccacatcgtgatggtggacgcctacaagcccaccaaatga

SEQ ID NO: 90 - DNA, 1082 bp, synthetic construct
  misc_feature 1..1082: note = pVAX1-SPYCEGFP (DNA)
  misc_feature 1..1082: note = pVAX1-SPYCEGFP
  source 1..1082: mol_type = other DNA
gagccatggtggatacactgtctggactgtctagcgagcagggccagagcggcgacatgacaatcgaggaagatagcgccacacacatcaagttcagcaagcgcgacgaggacggcaaagaactggctggcgctaccatggaactgagagacagcagcggcaagaccatcagcacctggatctctgacggccaagtgaaggacttctatctgtaccccggcaagtacaccttcgtggaaacagccgctcctgacggatacgaggtggccacagccatcaccttcaccgtgaacgagcagggacaagtgacagtgaacggcaaggccacaaagggcgacgctcacattggcggctctggctccatggtgtccaagggcgaagaactgttcaccggcgtggtgcccattctggtggaactggatggggatgtgaacggccacaagttctccgtgtctggcgaaggcgaaggggatgccacatacggcaagctgaccctgaagttcatctgcaccaccggaaagctgcccgtgccttggcctacactggtcaccacactgacatacggcgtgcagtgcttcagcagataccccgaccatatgaagcagcacgacttcttcaagagcgccatgcctgagggctacgtgcaagagagaaccatcttctttaaggacgacggcaactacaagaccagggccgaagtgaagttcgagggcgacaccctggtcaacagaatcgagctgaagggcatcgacttcaaagaggatggcaacatcctgggccacaagctcgagtacaactacaacagccacaacgtgtacatcatggccgacaagcagaaaaacggcatcaaagtgaacttcaagatccggcacaacatcgaggacggaagcgtgcagctggccgatcactaccagcagaacacacctatcggcgacggacctgtgctgctgcctgataaccactacctgagcacacagagcgccctgagcaaggaccctaacgagaagagggaccacatggtgctgctggaatttgtgaccgccgctggcatcacactcggcatggacgagctgtacaaatga

SEQ ID NO: 91 - protein, 287 aa, Myxococcus xanthus
MPDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERR

SEQ ID NO: 92 - protein, 428 aa, Geobacillus sp.
MAFEFKLPDIGEGIHEGEIVKWFVKPGDEVNEDDVLCEVQNDKAVVEIPSPVKGKVLEILVPEGTVATVGQMLITLDAPGYENMMFKGQEQEEAKKEEKTETVSKEEKVDAVAPNAPAAEAEAGPNRRVIAMPSVRKYAREKGVDIRLVQGTGKNGRVLKEDIDAFLAGGAKPAPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 93 - protein, 62 aa, Actinomyces viscosus
GSLSKYGKVILTKTGTDDLADKTKYNGAQFQVYECTKTASGATLRDSDPSTQTVDPLTIGGE

SEQ ID NO: 94 - DNA, 186 bp, Actinomyces viscosus
  source 1..186: mol_type = other DNA
ggcagcctgagcaaatatggcaaagtgattctgaccaaaaccggcaccgatgatctggcggataaaaccaaatataacggcgcgcagtttcaggtgtatgaatgcaccaaaaccgcgagcggcgcgaccctgcgcgatagcgatccgagcacccagaccgtggatccgctgaccattggcggcgaa

SEQ ID NO: 95 - protein, 102 aa, Streptococcus pneumoniae; identical to SEQ ID NO: 25

SEQ ID NO: 96 - DNA, 306 bp, Streptococcus pneumoniae
  source 1..306: mol_type = other DNA
ggcagcaccaccaaggtgaagctgatcaaggtggaccaggaccacaacaggctggagggcgtgggcttcaagctggtgagcgtggccagggacgtgagcgccgccgccgtgcccctgatcggcgagtacaggtacagcagcagcggccaggtgggcaggaccctgtacaccgacaagaacggcgagatcttcgtgaccaacctgcccctgggcaactacaggttcaaggaggtggagcccctggccggctacgccgtgaccaccctggacaccgacgtgcagctggtggaccaccagctggtgacc

SEQ ID NO: 97 - protein, 94 aa, Streptococcus pneumoniae; identical to SEQ ID NO: 26

SEQ ID NO: 98 - DNA, 282 bp, Streptococcus pneumoniae
  source 1..282: mol_type = other DNA
cccaggggcaacgtggacttcatgaaggtggacggcaggaccaacaccagcctgcagggcgccatgttcaaggtgatgaaggaggagagcggccactacacccccgtgctgcagaacggcaaggaggtggtggtgaccagcggcaaggacggcaggttcagggtggagggcctggagtacggcacctactacctgtgggagctgcaggcccccaccggctacgtgcagctgaccagccccgtgagcttcaccatcggcaaggacaccaggaaggagctggtg

SEQ ID NO: 99 - protein, 123 aa, Corynebacterium diphtheriae; identical to SEQ ID NO: 27

SEQ ID NO: 100 - DNA, 369 bp, Corynebacterium diphtheriae
  source 1..369: mol_type = other DNA
gtggtgacctaccacggcaagctgaaggtggtgaagaaggacggcaaggaggccggcaaggtgctgaagggcgccgagttcgagctgtaccagtgcaccagcgccgccgtgctgggcaagggccccctgaccgtggacggcgtgaagaagtggaccaccggcgacgacggcaccttcaccatcgacggcctgcacgtgaccgacttcgaggacggcaaggaggccgcccccgccaccaagaagttctgcctgaaggagaccaaggcccccgccggctacgccctgcccgaccccaacgtgaccgagatcgagttcaccagggccaagatcagcgagaaggacaagttcgagggcgacgacgaggtgacc

SEQ ID NO: 101 - protein, 134 aa, Lactobacillus rhamnosus; identical to SEQ ID NO: 28

SEQ ID NO: 102 - DNA, 402 bp, Lactobacillus rhamnosus
  source 1..402: mol_type = other DNA
agcaccaacgacaccaccacccagaacgtggtgctgaccaagtacggcttcgacaaggacgtgaccgccatcgacagggccaccgaccagatctggaccggcgacggcgccaagcccctgcagggcgtggacttcaccatctacaacgtgaccgccaactactgggccagccccaaggactacaagggcagcttcgacagcgcccccgtggccgccaccggcaccaccaacgacaagggccagctgacccaggccctgcccatccagagcaaggacgccagcggcaagaccagggccgccgtgtacctgttccacgagaccaaccccagggccggctacaacaccagcgccgacttctggctgaccctgcccgccaaggccgccgccgacggcaacgtgtac

SEQ ID NO: 103 - protein, 114 aa, Lactobacillus rhamnosus; identical to SEQ ID NO: 29

SEQ ID NO: 104 - DNA, 342 bp, Lactobacillus rhamnosus
  source 1..342: mol_type = other DNA
accacctacgagaggaccttcgtgaagaaggacgccgagaccaaggaggtgctggagggcgccggcttcaagatcagcaacagcgacggcaagttcctgaagctgaccgacaaggacggccagagcgtgagcatcggcgagggcttcatcgacgtgctggccaacaactacaggctgacctgggtggccgagagcgacgccaccgtgttcaccagcgacaagagcggcaagttcggcctgaacggcttcgccgacaacaccaccacctacaccgccgtggagaccaacgtgcccgacggctacgacgccgccgccaacaccgacttcaaggccgacaacagc

SEQ ID NO: 105 - protein, 107 aa, Streptococcus agalactiae; identical to SEQ ID NO: 30

SEQ ID NO: 106 - DNA, 321 bp, Streptococcus agalactiae
  source 1..321: mol_type = other DNA
ggccagatcaccatcaagaagatcgacggcagcaccaaggccagcctgcagggcgccatcttcgtgctgaagaacgccaccggccagttcctgaacttcaacgacaccaacaacgtggagtggggcaccgaggccaacgccaccgagtacaccaccggcgccgacggcatcatcaccatcaccggcctgaaggagggcacctactacctggtggagaagaaggcccccctgggctacaacctgctggacaacagccagaaggtgatcctgggcgacggcgccaccgacaccaccaacagcgacaacctgctggtgaacccc

SEQ ID NO: 107 - protein, 91 aa, Streptococcus intermedius; identical to SEQ ID NO: 31

SEQ ID NO: 108 - DNA, 273 bp, Streptococcus intermedius
  source 1..273: mol_type = other DNA
gagcaggacgtggtgttcagcaaggtgaacgtggccggcgaggagatcgccggcgccaagatccagctgaaggacgcccagggccaggtggtgcacagctggaccagcaaggccggccagagcgagaccgtgaagctgaaggccggcacctacaccttccacgaggccagcgcccccaccggctacctggccgtgaccgacatcaccttcgaggtggacgtgcagggcaaggtgaccgtgaaggacgccaacggcaacggcgtgaaggccgac

SEQ ID NO: 109 - protein, 104 aa, Streptococcus pneumoniae; identical to SEQ ID NO: 32

SEQ ID NO: 110 - DNA, 312 bp, Streptococcus pneumoniae
  source 1..312: mol_type = other DNA
aagctgggcgacatcgagttcatcaaggtgaacaagaacgacaagaagcccctgaggggcgccgtgttcagcctgcagaagcagcaccccgactaccccgacatctacggcgccatcgaccagaacggcacctaccagaacgtgaggaccggcgaggacggcaagctgaccttcaagaacctgagcgacggcaagtacaggctgttcgagaacagcgagcccgccggctacaagcccgtgcagaacaagcccatcgtggccttccagatcgtgaacggcgaggtgagggacgtgaccagcatcgtgccccag

SEQ ID NO: 111 - protein, 130 aa, Corynebacterium diphtheriae; identical to SEQ ID NO: 33

SEQ ID NO: 112 - DNA, 390 bp, Corynebacterium diphtheriae
  source 1..390: mol_type = other DNA
ggcagcgagaggaagggcagcctgaccctgcacaagaagaagggcgccgagagcgagaagagggccaccggcaaggagatggacgacgtggccggcgagcccctgaacggcgtgaccttcaagatcaccaagctgaacttcgacctgcagaacggcgactgggccaagttccccaagaccgccgccgacgccaagggccacgagaccagcaccaccaaggaggtggagaccagcggcaacggcaccgccgtgttcgacaacctggacctgggcatctacctggtggaggagaccaaggcccccgacggcatcgtgaccggcgcccccttcatcgtgagcatccccatggtgaacgaggccagcgacgcctggaactacaacgtggtggcc

SEQ ID NO: 113 - protein, 113 aa, synthetic construct
  REGION 1..113: note = QueenCatcher
  REGION 1..113: note = MISC_FEATURE - QueenCatcher
IDTMSGLSGETGQSGNTTIEEDSTTHVKFSKRDSNGKELAGAMIELRNLSGQTIQSWVSDGTVKDFYLMPGTYQFVETAAPEGYELAAPITFTVNEQGQVTVNGKATKGDAHI

SEQ ID NO: 114 - DNA, 339 bp, synthetic construct
  misc_feature 1..339: note = QueenCatcher DNA
  misc_feature 1..339: note = QueenCatcher
  source 1..339: mol_type = other DNA
atcgacaccatgagcggcctgagcggcgagaccggccagagcggcaacaccaccatcgaggaggacagcaccacccacgtgaagttcagcaagagggacagcaacggcaaggagctggccggcgccatgatcgagctgaggaacctgagcggccagaccatccagagctgggtgagcgacggcaccgtgaaggacttctacctgatgcccggcacctaccagttcgtggagaccgccgcccccgagggctacgagctggccgcccccatcaccttcaccgtgaacgagcagggccaggtgaccgtgaacggcaaggccaccaagggcgacgcccacatc

SEQ ID NO: 115 - protein, 113 aa, synthetic construct
  REGION 1..113: note = MoonCake
  REGION 1..113: note = MISC_FEATURE - MoonCake
IDTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVIIQGRLTRGDVHI

SEQ ID NO: 116 - DNA, 339 bp, synthetic construct
  misc_feature 1..339: note = MoonCake DNA
  misc_feature 1..339: note = MoonCake
  source 1..339: mol_type = other DNA
atcgacaccatgagcggcctgagcggcgagaccggccagagcggcaacaccaccatcgaggaggacagcaccacccacgtgaagttcagcaagagggacagcaacggcaaggagctggccggcgccatgatcgagctgaggaacctgagcggccagaccatccagagctgggtgagcgacggcaccgtgaaggacttctacctgatgcccggcacctaccagttcgtggagaccgccgcccccgagggctacgagctggccgcccccatcaccttcaccgtgcaggacaacggcgaggtgatcatccagggcaggctgaccaggggcgacgtgcacatc

SEQ ID NO: 117 - protein, 113 aa, synthetic construct
  REGION 1..113: note = Kat I
  REGION 1..113: note = MISC_FEATURE - Kat I
IDTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVQIQGKATRGDVPI

SEQ ID NO: 118 - DNA, 339 bp, synthetic construct
  misc_feature 1..339: note = Kat I DNA
  misc_feature 1..339: note = Kat I
  source 1..339: mol_type = other DNA
atcgacaccatgagcggcctgagcggcgagaccggccagagcggcaacaccaccatcgaggaggacagcaccacccacgtgaagttcagcaagagggacagcaacggcaaggagctggccggcgccatgatcgagctgaggaacctgagcggccagaccatccagagctgggtgagcgacggcaccgtgaaggacttctacctgatgcccggcacctaccagttcgtggagaccgccgcccccgagggctacgagctggccgcccccatcaccttcaccgtgcaggacaacggcgaggtgcagatccagggcaaggccaccaggggcgacgtgcccatc

SEQ ID NO: 119 - protein, 14 aa, synthetic construct
  REGION 1..14: note = Clib9
  REGION 1..14: note = MISC_FEATURE - Clib9
VPTIVMVDCYKRYK

SEQ ID NO: 120 - DNA, 42 bp, synthetic construct
  misc_feature 1..42: note = Clib9 DNA
  misc_feature 1..42: note = Clib9
  source 1..42: mol_type = other DNA
gtgcccaccatcgtgatggtggactgctacaagaggtacaag

SEQ ID NO: 121 - protein, 15 aa, synthetic construct
  REGION 1..15: note = TLR4 agonist peptide
  REGION 1..15: note = MISC_FEATURE - TLR4 agonist peptide
RYETMSIMIKSGGKY

SEQ ID NO: 122 - DNA, 45 bp, synthetic construct
  misc_feature 1..45: note = TLR4 agonist peptide DNA
  misc_feature 1..45: note = TLR4 agonist
  source 1..45: mol_type = other DNA
aggtacgagaccatgagcatcatgatcaagagcggcggcaagtac

SEQ ID NO: 123 - protein, 16 aa, Mycobacterium tuberculosis
CSSNKSTTGSGETTTA

SEQ ID NO: 124 - DNA, 48 bp, Mycobacterium tuberculosis
  source 1..48: mol_type = other DNA
tgcagcagcaacaagagcaccaccggcagcggcgagaccaccaccgcc

SEQ ID NO: 125 - protein, 28 aa, Mus musculus
KFLNTAKDRNRWEEPDQQLYNVEATSYA

SEQ ID NO: 126 - DNA, 84 bp, Mus musculus
  source 1..84: mol_type = other DNA
aagttcctgaacaccgccaaggacaggaacaggtgggaggagcccgaccagcagctgtacaacgtggaggccaccagctacgcc

SEQ ID NO: 127 - protein, 12 aa, synthetic construct
  REGION 1..12: note = Peptide binding to Dendritic cell receptor
  REGION 1..12: note = MISC_FEATURE - Peptide binding to dendritic cell receptor
NWYLPWLGTNDW

SEQ ID NO: 128 - DNA, 36 bp, synthetic construct
  misc_feature 1..36: note = Peptide binding to Dendritic cell receptor (DNA)
  misc_feature 1..36: note = peptide binding to dendritic cell receptor - DNA sequence
  source 1..36: mol_type = other DNA
aactggtacctgccctggctgggcaccaacgactgg

SEQ ID NO: 129 - protein, 14 aa, Vespula lewisii
INLKALAALAKALL

SEQ ID NO: 130 - DNA, 42 bp, Vespula lewisii
  source 1..42: mol_type = other DNA
atcaacctgaaggccctggccgccctggccaaggccctgctg

SEQ ID NO: 131 - protein, 14 aa, Vespula lewisii
INLKALAALAKKIL

SEQ ID NO: 132 - DNA, 42 bp, Vespula lewisii
  source 1..42: mol_type = other DNA
atcaacctgaaggccctggccgccctggccaagaagatcctg

SEQ ID NO: 133 - protein, 9 aa, synthetic construct
  REGION 1..9: note = TLR2-activating peptide
  REGION 1..9: note = MISC_FEATURE - consensus sequence
  VARIANT 6: note = X is any amino acid
  VARIANT 3: note = X is Ser o Thr
GCXGLXSIT

SEQ ID NO: 134 - DNA, 27 bp, synthetic construct
  misc_feature 1..27: note = TLR2-activating peptide (DNA)
  misc_feature 1..27: note = TLR2-activating peptide (DNA) example
  source 1..27: mol_type = other DNA
ggctgcagcggcctgaccagcatcacc

SEQ ID NO: 135 - protein, 5 aa, synthetic construct
  REGION 1..5: note = TH2-biasing Synthetic peptide 1
  REGION 1..5: note = MISC_FEATURE - TH2-biasing Synthetic peptide 1
KWCEC

SEQ ID NO: 136 - DNA, 15 bp, synthetic construct
  misc_feature 1..15: note = TH2-biasing Synthetic peptide 1 (DNA)
  misc_feature 1..15: note = TH2-biasing Synthetic peptide 1 DNA
  source 1..15: mol_type = other DNA
aagtggtgcgagtgc

SEQ ID NO: 137 - protein, 12 aa, synthetic construct
  REGION 1..12: note = TH2-biasing Synthetic peptide 2
  REGION 1..12: note = MISC_FEATURE - TH2-biasing Synthetic peptide 2
KWCECKFFKFFG

SEQ ID NO: 138 - DNA, 36 bp, synthetic construct
  misc_feature 1..36: note = TH2-biasing Synthetic peptide 2 (DNA)
  misc_feature 1..36: note = TH2-biasing Synthetic peptide 2 DNA
  source 1..36: mol_type = other DNA
aagtggtgcgagtgcaagttcttcaagttcttcggc

SEQ ID NO: 139 - protein, 12 aa, synthetic construct
  REGION 1..12: note = TH2-biasing Synthetic peptide 3
  REGION 1..12: note = MISC_FEATURE - TH2-biasing Synthetic peptide 3
KWCECEFFEFFG

SEQ ID NO: 140 - DNA, 36 bp, synthetic construct
  misc_feature 1..36: note = TH2-biasing Synthetic peptide 3 (DNA)
  misc_feature 1..36: note = TH2-biasing Synthetic peptide 3 DNA
  source 1..36: mol_type = other DNA
aagtggtgcgagtgcgagttcttcgagttcttcggc

SEQ ID NO: 141 - protein, 261 aa, Pantoea sp.
AQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDGGSPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 142 - DNA, 783 bp, Pantoea sp.
  source 1..783: mol_type = other DNA
gcacaggtgattaataccaatagcctgagcctgctgacccagaataatctgaataaaagccagagcgcactgggcaccgcaattgaacgtctgagcagcggtctgcgtattaatagcgccaaagatgatgcagccggtcaggcaattgcaaatcgttttaccgcaaacattaaaggtctgacccaggcaagccgtaatgcaaatgatggtattagcattgcacagaccaccgaaggtgcactgaatgaaattaacaataatctgcagcgtgttcgtgaactggccgttcagagcgcaaatggtacaaatagccagagtgatctggatagcattcaggcagaaattacccagcgtctgaacgaaattgatcgtgttagcggtcagacccagtttaatggtgttaaagttctggcacaggataacaccctgaccattcaggttggtgccaatgatggcgaaaccattgatattgatctgaaagagattagcagcaaaaccctgggtttagatggtggtagtccgctgcagaaaattgatgcagcactggcccaggttgataccctgcgtagcgatctgggtgcagttcagaatcgctttaatagtgcaattaccaatctgggcaataccgttaataatctgtcaagcgcacgtagccgtattgaagatagcgattatgcaaccgaagttagcaatatgagccgtgcacagattctgcagcaggcaggcaccagcgttctggcgcaggcaaatcaggttccgcagaatgttctgagtctgctgcgt

SEQ ID NO: 143 - protein, 131 aa, Acinetobacter phage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 144 - DNA, 393 bp, Acinetobacter phage AP205
  source 1..393: mol_type = other DNA
atggcgaacaaaccgatgcagccgattaccagcaccgcgaacaaaattgtgtggagcgatccgacccgcctgagcaccacctttagcgcgagcctgctgcgccagcgcgtgaaagtgggcattgcggaactgaacaacgtgagcggccagtatgtgagcgtgtataaacgcccggcgccgaaaccggaaggctgcgcggatgcgtgcgtgattatgccgaacgaaaaccagagcattcgcaccgtgattagcggcagcgcggaaaacctggcgaccctgaaagcggaatgggaaacccataaacgcaacgtggataccctgtttgcgagcggcaacgcgggcctgggctttctggatccgaccgcggcgattgtgagcagcgataccaccgcg

SEQ ID NO: 145 - protein, 273 aa, unidentified
  REGION 1..273: note = SARS-CoV-2 spike protein RBD
  REGION 1..273: note = MISC_FEATURE - SARS-CoV-2 spike protein RBD
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 146 - DNA, 819 bp, unidentified
  misc_feature 1..819: note = DNA sequence for SARS-CoV-2 spike protein RBD
  misc_feature 1..819: note = DNA sequence for SARS-CoV-2 spike protein RBD
  source 1..819: mol_type = other DNA
cgcgtgcagcccaccgagagcatcgtgcgcttccccaacatcaccaacctgtgccccttcggcgaggtgttcaacgccacccgcttcgccagcgtgtacgcctggaaccgcaagcgcatcagcaactgcgtggccgattacagcgtgctgtacaacagcgccagcttcagcaccttcaagtgctacggcgtgagccccaccaagctgaacgatctgtgcttcaccaacgtgtacgccgatagcttcgtgatccgcggcgatgaggtgcgccagatcgcccccggccagaccggcaagatcgccgattacaactacaagctgcccgatgatttcaccggctgcgtgatcgcctggaacagcaacaacctggatagcaaggtgggcggcaactacaactacctgtaccgcctgttccgcaagagcaacctgaagcccttcgagcgcgatatcagcaccgagatctaccaggccggcagcaccccctgcaacggcgtggagggcttcaactgctacttccccctgcagagctacggcttccagcccaccaacggcgtgggctaccagccctaccgcgtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggccgcgatatcgccgataccaccgatgccgtgcgcgatccccagaccctggagatcctggatatcaccccctgcagc

SEQ ID NO: 147 - protein, 98 aa, Streptococcus pyogenes
DYDGQSGDGKELAGATMELRDSSGKTISTWISDGQVKDFYLYPGKYTFVETAAPDGYEVATAITFTVNEQGQVTVNGKATKGGSGGSGGSGEDSATHI

SEQ ID NO: 148 - DNA, 294 bp, Streptococcus pyogenes
  source 1..294: mol_type = other DNA
gattacgacggtcagtccggtgacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgggagtggtggcagcggaggtagtggcgaggacagcgctacccatatt

SEQ ID NO: 149 - protein, 244 aa, synthetic construct
  REGION 1..244: note = Scfv DEC-205
  REGION 1..244: note = misc_feature - Scfv DEC-205
EVKLQQSGTEVVKPGASVKLSCKASGYIFTSYDIDWVRQTPEQGLEWIGWIFPGEGSTEYNEKFKGRATLSVDKSSSTAYMELTRLTSEDSAVYFCARGDYYRRYFDLWGQGTTVTVSSGGGGSGGGGSGGGGSDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLEIKRAA

SEQ ID NO: 150 - DNA, 732 bp, synthetic construct
  misc_feature 1..732: note = Scfv DEC-205 (DNA)
  misc_feature 1..732: note = Scfv DEC-205 DNA
  source 1..732: mol_type = other DNA
gaagtgaagctgcagcagagcggaaccgaggttgtgaagccaggtgccagtgtgaagctgagctgcaaggccagcggctacatcttcaccagctacgacatcgattgggtccgacagaccccagagcaaggcctggaatggatcggctggattttcccaggcgagggcagcaccgagtacaacgagaagtttaagggacgcgccacgctgtccgtggataagagtagtagcaccgcctacatggaactgacccgcctgacaagcgaagatagcgccgtgtacttttgcgcccgtggcgattactaccgccgctactttgatctgtggggccagggaaccaccgtgacagttagttcaggtggcggaggtagcggaggtggtggatcaggtggtggcggaagcgatattcagatgacccagtcgccgagcttcctgagcacaagcctgggcaacagcatcaccattacctgccacgccagccagaacatcaaaggctggctggcctggtatcagcagaagtccggaaacgccccacagctgctgatctacaaggctagcagcctgcaaagcggagtgccaagccgttttagcggaagtggaagcggcaccgattacattttcaccatcagcaatctgcagcccgaggacattgccacctactactgccagcactaccagagcttcccgtggacattcggcggaggcaccaagctggaaatcaagcgtgctgcc

SEQ ID NO: 151 - protein, 265 aa, Thermotoga maritima [sequence omitted]

SEQ ID NO: 152 - protein, 345 aa, Pyrococcus furiosus
MLSINPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLE

SEQ ID NO: 153 - protein, 15 aa, Escherichia coli
GLNDIFEAQKIEWHE

SEQ ID NO: 154 - DNA, 45 bp, Escherichia coli
  source 1..45: mol_type = other DNA
ggcctgaacgacatcttcgaggcccagaagatcgagtggcacgag

SEQ ID NO: 155 - protein, 121 aa, Streptomyces avidinii
AEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGS

SEQ ID NO: 156 - DNA, 363 bp, Streptomyces avidinii
  source 1..363: mol_type = other DNA
gccgaggccggcatcaccggcacctggtacaaccagcacggcagcaccttcaccgtgaccgccggcgccgacggcaacctgaccggccagtacgagaacagggcccagggcaccggctgccagaacagcccctacaccctgaccggcaggtacaacggcaccaagctggagtggagggtggagtggaacaacagcaccgagaactgccacagcaggaccgagtggaggggccagtaccagggcggcgccgaggccaggatcaacacccagtggaacctgacctacgagggcggcagcggccccgccaccgagcagggccaggacaccttcaccaaggtgaagcccagcgccgccagcggcagc

SEQ ID NO: 157 - protein, 215 aa, synthetic construct
  REGION 1..215: note = I53-50A
  REGION 1..215: note = misc_feature - I53-50A
EKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 158 - DNA, 645 bp, synthetic construct
  misc_feature 1..645: note = I53-50A (DNA)
  misc_feature 1..645: note = I53-50A (DNA)
  source 1..645: mol_type = other DNA
gaaaaagcggcgaaagcggaagaagcggcgcgcaaaatggaagaactgtttaaaaaacataaaattgtggcggtgctgcgcgcgaacagcgtggaagaagcgattgaaaaagcggtggcggtgtttgcgggcggcgtgcatctgattgaaattacctttaccgtgccggatgcggataccgtgattaaagcgctgagcgtgctgaaagaaaaaggcgcgattattggcgcgggcaccgtgaccagcgtggaacaggcgcgcaaagcggtggaaagcggcgcggaatttattgtgagcccgcatctggatgaagaaattagccagtttgcgaaagaaaaaggcgtgttttatatgccgggcgtgatgaccccgaccgaactggtgaaagcgatgaaactgggccataccattctgaaactgtttccgggcgaagtggtgggcccgcagtttgtgaaagcgatgaaaggcccgtttccgaacgtgaaatttgtgccgaccggcggcgtgaacctggataacgtggcggaatggtttaaagcgggcgtgctggcggtgggcgtgggcagcgcgctggtgaaaggcaccccggatgaagtgcgcgaaaaagcgaaagcgtttgtggaaaaaattcgcggcgcgaccgaa

SEQ ID NO: 159 - protein, 131 aa, synthetic construct
  REGION 1..131: note = I53-50B
  REGION 1..131: note = misc_feature - I53-50B
MDVNMKLTGRIMDAAKEVDHTCRSSTGVPRDMLHRYAEGQTVDDDDFKCYLKCIMVEFNSLSDDGVFVLEEELENVPPEIKEEGHRVVHSCKHINHDEACETAYQIHQCYKQSDPELYSLVVRAFDATIGD

SEQ ID NO: 160 - DNA, 393 bp, synthetic construct
  misc_feature 1..393: note = I53-50B (DNA)
  misc_feature 1..393: note = I53-50B (DNA)
  source 1..393: mol_type = other DNA
atggatgtgaacatgaaactgaccggccgcattatggatgcggcgaaagaagtggatcatacctgccgcagcagcaccggcgtgccgcgcgatatgctgcatcgctatgcggaaggccagaccgtggatgatgatgattttaaatgctatctgaaatgcattatggtggaatttaacagcctgagcgatgatggcgtgtttgtgctggaagaagaactggaaaacgtgccgccggaaattaaagaagaaggccatcgcgtggtgcatagctgcaaacatattaaccatgatgaagcgtgcgaaaccgcgtatcagattcatcagtgctataaacagagcgatccggaactgtatagcctggtggtgcgcgcgtttgatgcgaccattggcgat

SEQ ID NO: 161 - protein, 13 aa, Streptococcus intermedius
GNKLTVTDQAAPS

SEQ ID NO: 162 - DNA, 39 bp, Streptococcus intermedius
  source 1..39: mol_type = other DNA
ggcaacaagctgaccgtgaccgaccaggccgcccccagc

SEQ ID NO: 163 - protein, 10 aa, Streptococcus pyogenes
ATHIKFSKRD

SEQ ID NO: 164 - DNA, 30 bp, Streptococcus pyogenes
  source 1..30: mol_type = other DNA
gccacccacatcaagttcagcaagagggac

SEQ ID NO: 165 - protein, 23 aa, Streptococcus pneumoniae
DIPATYEFTDGKHYITNEPIPPK

SEQ ID NO: 166 - protein, 107 aa, Streptococcus pneumoniae
GAMKLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 167 - protein, 12 aa, synthetic construct
  REGION 1..12: note = CL3
  REGION 1..12: note = misc_feature - CL3
GWKERLSSWNRF

SEQ ID NO: 168 - protein, 12 aa, synthetic construct
  REGION 1..12: note = Co1
  REGION 1..12: note = misc_feature - Co1
SFHQLPARSPLP

SEQ ID NO: 169 - protein, 9 aa, synthetic construct
  REGION 1..9: note = CKS9
  REGION 1..9: note = misc_feature - CKS9
CKSTHPLSC

SEQ ID NO: 170 - protein, 12 aa, synthetic construct
  REGION 1..12: note = Peptide 3
  REGION 1..12: note = misc_feature - Peptide 3
FYPSYHSTPQRP

SEQ ID NO: 171 - protein, 12 aa, synthetic construct
  REGION 1..12: note = Peptide 12
  REGION 1..12: note = misc_feature - Peptide 12
AYYKTASLAPAE

SEQ ID NO: 172 - protein, 12 aa, synthetic construct
  REGION 1..12: note = Peptide 18
  REGION 1..12: note = misc_feature - Peptide 18
SLSLLTMPGNAS

SEQ ID NO: 173 - protein, 7 aa, synthetic construct
  REGION 1..7: note = IRMM_01
  REGION 1..7: note = misc_feature - IRMM_01
YTYQGKL

SEQ ID NO: 174 - protein, 9 aa, synthetic construct
  REGION 1..9: note = IRMM_02
  REGION 1..9: note = misc_feature - IRMM_02
CYTYQGKLC

SEQ ID NO: 175 - protein, 31 aa, Homo sapiens
LNCSNCPQPQNSSLRTPLRQGGSAGSAGSAG

SEQ ID NO: 176 - protein, 39 aa, Homo sapiens
SLERFTGLDLANVTLTYEFAAGPRDFWQPGSAGSAGSAG

SEQ ID NO: 177 - protein, 20 aa, synthetic construct
  REGION 1..20: note = p30
  REGION 1..20: note = misc_feature - p30
GSGSGSGSCGRWSGWPADLC

SEQ ID NO: 178 - protein, 12 aa, synthetic construct
  REGION 1..12: note = PCP1
  REGION 1..12: note = misc_feature - PCP1
HSIFTAFPGSRP

SEQ ID NO: 179 - protein, 12 aa, synthetic construct
  REGION 1..12: note = PCP3
  REGION 1..12: note = misc_feature - PCP3
ATYSEFPGNLKP

SEQ ID NO: 180 - protein, 12 aa, synthetic construct
  REGION 1..12: note = PCP4
  REGION 1..12: note = misc_feature - PCP4
SMPMAYIPFPGN

SEQ ID NO: 181 - protein, 20 aa, synthetic construct
  REGION 1..20: note = XS52.1
  REGION 1..20: note = misc_feature - XS52.1
GPEDTSRAPENQQKTFHRRW

SEQ ID NO: 182 - protein, 9 aa, synthetic construct
  REGION 1..9: note = IRMM_03
  REGION 1..9: note = misc_feature - IRMM_03
FGPNLTGRW

SEQ ID NO: 183 - protein, 9 aa, synthetic construct
  REGION 1..9: note = IRMM_04
  REGION 1..9: note = misc_feature - Consensus
  VARIANT 7: note = X = M/L
  VARIANT 8: note = X=Any amino acid
  VARIANT 9: note = X=V/I/L
  VARIANT 3: note = X=Any amino acid
DLXTSKXXX

SEQ ID NO: 184 - protein, 6 aa, synthetic construct
  REGION 1..6: note = IRMM_05
  REGION 1..6: note = misc_feature - IRMM_05
HFYLPM

SEQ ID NO: 185 - protein, 6 aa, synthetic construct
  REGION 1..6: note = IRMM_06
  REGION 1..6: note = misc_feature - IRMM_06
MFYLPM

SEQ ID NO: 186 - protein, 6 aa, synthetic construct
  REGION 1..6: note = IRMM_07
  REGION 1..6: note = misc_feature - Consensus
  VARIANT 5: note = X=P/V/Y
  VARIANT 1: note = X=K/R/M
XKYYXM

SEQ ID NO: 187 - protein, 4 aa, synthetic construct
  REGION 1..4: note = C-tag purification tag
  REGION 1..4: note = misc_feature - C-tag purification tag
EPEA

SEQ ID NO: 188 - protein, 4 aa, synthetic construct
  REGION 1..4: note = Linker_01
  REGION 1..4: note = misc_feature - Linker_01
GSGS

SEQ ID NO: 189 - protein, 11 aa, synthetic construct
  REGION 1..11: note = Linker_02
  REGION 1..11: note = misc_feature - Linker_02
GSGTAGGGSGS

SEQ ID NO: 190 - protein, 5 aa, synthetic construct
  REGION 1..5: note = Linker
  REGION 1..5: note = MISC_FEATURE - Linker sequence
GGSGS

SEQ ID NO: 191 - protein, 10 aa, synthetic construct
  REGION 1..10: note = Linker
  REGION 1..10: note = MISC_FEATURE - Linker
GTASGGSGGS

SEQ ID NO: 192 - protein, 272 aa, synthetic construct
  REGION 1..272: note = Example construct
  REGION 1..116: note = MISC_FEATURE - SpyCatcher
  REGION 117..121: note = MISC_FEATURE - Linker
  REGION 122..252: note = MISC_FEATURE - AP205
  REGION 253..257: note = MISC_FEATURE - Linker
  REGION 258..272: note = MISC_FEATURE - TLR4 agonist
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGSGSMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGGSGSRYETMSIMIKSGGKY

SEQ ID NO: 193 - protein, 264 aa, synthetic construct
  REGION 1..264: note = Example construct
  REGION 1..116: note = MISC_FEATURE - SpyCatcher
  REGION 117..121: note = MISC_FEATURE - Linker
  REGION 122..252: note = MISC_FEATURE - AP205
  REGION 253..257: note = MISC_FEATURE - Linker
  REGION 258..264: note = MISC_FEATURE - TLR4 agonist peptide
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGSGSMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGGSGSAPPHALS

SEQ ID NO: 194 - protein, 252 aa, synthetic construct
  REGION 1..252: note = Example construct
  REGION 1..116: note = MISC_FEATURE - SpyCatcher
  REGION 117..121: note = MISC_FEATURE - Linker
  REGION 122..252: note = MISC_FEATURE - AP205
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGSGSMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 195 - protein, 183 aa, synthetic construct
  REGION 1..183: note = Example construct
  REGION 1..16: note = MISC_FEATURE - CD180 agonist
  REGION 17..27: note = MISC_FEATURE - Linker
  REGION 28..158: note = MISC_FEATURE - AP205
  REGION 159..168: note = MISC_FEATURE - Linker
  REGION 169..183: note = MISC_FEATURE - RumTrunkD9N
CSSNKSTTGSGETTTAGSGTAGGGSGSMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGTASGGSGGSGNPLIVMVNDTTKVK

SEQ ID NO: 196 - protein, 195 aa, synthetic construct
  REGION 1..195: note = Example construct
  REGION 1..15: note = MISC_FEATURE - Rumtrunk D9N
  REGION 16..26: note = MISC_FEATURE - Linker
  REGION 27..157: note = MISC_FEATURE - AP205
  REGION 158..167: note = MISC_FEATURE - Linker
  REGION 168..195: note = MISC_FEATURE - C3d complement peptide
GNPLIVMVNDTTKVKGSGTAGGGSGSMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGTASGGSGGSKFLNTAKDRNRWEEPDQQLYNVEATSYA

SEQ ID NO: 197 - protein, 386 aa, synthetic construct
  REGION 1..386: note = Example construct
  REGION 1..113: note = MISC_FEATURE - MoonCake
  REGION 114..386: note = MISC_FEATURE - RBD
IDTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVIIQGRLTRGDVHIRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 198 - protein, 11 aa, synthetic construct
AXAAAATLKAA

SEQ ID NO: 199 - protein, 11 aa, synthetic construct
AXAAAKTAAAA

SEQ ID NO: 200 - protein, 11 aa, synthetic construct
AXIAAATLKAA

SEQ ID NO: 201 - protein, 12 aa, synthetic construct
AAXVAAATLKAA

SEQ ID NO: 202 - protein, 11 aa, synthetic construct
KXVAAWTLKAA

SEQ ID NO: 203 - protein, 11 aa, synthetic construct
KFVAAWTLKAA

SEQ ID NO: 204 - protein, 70 aa, Homo sapiens
APLAADTPTACCFSYTSRQIPQNFIADYFETSSQCSKPSVIFLTKRGRQVCADPSEEWVQKYVSDLELSA

SEQ ID NO: 205 - protein, 7 aa, synthetic construct
APPHALS

SEQ ID NO: 206 - protein, 11 aa, Homo sapiens
ATPEDNGRSFS

SEQ ID NO: 207 - protein, 12 aa, synthetic construct
CGRWSGWPADLC

SEQ ID NO: 208 - protein, 21 aa, Plasmodium falciparum
DIEKKIAKMEKASSVFNVVNS

SEQ ID NO: 209 - protein, 60 aa, synthetic construct
DNNSLSQEVQNGSNHLENNQSQSNGGGSDSALSLSSKTAALAAATTVNDGSDGATSSAVG

SEQ ID NO: 210 - protein, 38 aa, synthetic construct
EVDAPPHALSGGGAPPHALSGGGAPPHALSVDKLGDLG

SEQ ID NO: 211 - protein, 66 aa, synthetic construct
EVDAPPHALSGGGAPPHALSGGGAPPHALSVDKLQEINSSYGGGQEINSSYGGGQEINSSYKGDLG

SEQ ID NO: 212 - protein, 9 aa, Tannerella forsythia
GCSGLTSIT

SEQ ID NO: 213 - protein, 9 aa, Tannerella forsythia
GCTGLTSIT

SEQ ID NO: 214 - protein, 7 aa, synthetic construct
GETRAPL

SEQ ID NO: 215 - protein, 17 aa, synthetic construct
GPEDTSRAPENQQKTGC

SEQ ID NO: 216 - protein, 28 aa, Homo sapiens
KFLTTAKDKNRWEDPGKQLYNVEATSYA

SEQ ID NO: 217 - protein, 21 aa, Homo sapiens
LNCSNCPQPQNSSLRTPLRQG

SEQ ID NO: 218 - protein, 24 aa, Homo sapiens
LPKPPKPVSKMRMATPLLMGALPM

SEQ ID NO: 219 - protein, 6 aa, synthetic construct
MKYYYM

SEQ ID NO: 220 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 221 - protein, 90 aa, synthetic construct
NIDRPKGLAFTDVDVDSIKIAWESPQGQVSRYRVTYSSPEDGIRELFPAPDGEDDTAELQGLRPGSEYTVSVVALHDDMESQPLIGIQST

SEQ ID NO: 222 - protein, 20 aa, Hepatitis B virus
PHHTALRQAILCWGELMTLA

SEQ ID NO: 223 - protein, 13 aa, Influenza A virus
PKYVKQNTLKLAT

SEQ ID NO: 224 - protein, 13 aa, Homo sapiens
PQPQNSSLRTPLR

SEQ ID NO: 225 - protein, 7 aa, synthetic construct
QLPRTAL

SEQ ID NO: 226 - protein, 12 aa, synthetic construct
QVNFPPSKYPPT

SEQ ID NO: 227 - protein, 12 aa, synthetic construct
QWELPWLMQPPL

SEQ ID NO: 228 - protein, 14 aa, Clostridium tetani
QYIKANSKFIGITE

SEQ ID NO: 229 - protein, 30 aa, Clostridium perfringens
SLDAGQYVLVMKANSSYSGNYPYSILFQKF

SEQ ID NO: 230 - protein, 29 aa, Homo sapiens
SLERFTGLDLANVTLTYEFAAGPRDFWQP

SEQ ID NO: 231 - protein, 12 aa, synthetic construct
SPGLSLVSHMQT

SEQ ID NO: 232 - protein, 35 aa, Mus musculus
STVHEILCKLSLEGDHSTPPSAYGSVKPYTNFDAE

SEQ ID NO: 233 - protein, 7 aa, synthetic construct
TPAFRYS

SEQ ID NO: 234 - protein, 9 aa, Homo sapiens
VQGEESNDK

SEQ ID NO: 235 - protein, 13 aa, Homo sapiens
VTLTYEFAAGPRD

SEQ ID NO: 236 - protein, 16 aa, Influenza A virus
YSGPLKAEIAQRLEDV

SEQ ID NO: 237 - protein, 15 aa, synthetic construct
RYETMSIMIKSGGKG

SEQ ID NO: 238 - protein, 8 aa, synthetic construct
EWGWRMII

SEQ ID NO: 239 - protein, 17 aa, synthetic construct
PLWMMIKSMGSMMEMKK

SEQ ID NO: 240 - protein, 9 aa, synthetic construct
ILYMSLKWM

SEQ ID NO: 241 - protein, 9 aa, synthetic construct
ILYKSLKWM

SEQ ID NO: 242 - protein, 70 aa, Homo sapiens
ASLAADTPTACCFSYTSRQIPQNFIADYFETSSQCSKPGVIFLTKRSRQVCADPSEEWVQKYVSDLELSA

(19) United States
(12) Patent Application Publication
(10) Pub. No.: US 2025/0161437 A1
(43) Pub. Date: May 22, 2025

(54) METHODS AND PARTICLES FOR MODULATING AN IMMUNE RESPONSE

(71) Applicant: ADAPTVAC APS, Copenhagen N (DK)

(72) Inventors: Adam Frederik Sander BERTELSEN, Dragør (DK); Cyrielle Elyette FOUGEROUX, Copenhagen S (DK); Willem Adriaan DE JONGH, Manchester (GB)

(21) Appl. No.: 18/696,280

(22) PCT Filed: Sep. 30, 2022
(86) PCT No.: PCT/EP2022/077310

§ 371 Date: Mar. 27, 2024

(30) Foreign Application Priority Data
Sep. 30, 2021 (EP) 21200186.1

(51) Int. Cl. A61K 39/385; A61K 39/00; A61K 39/02; A61K 39/05; A61K 39/07; A61K 39/08; A61K 39/09; A61K 39/215; A61K 47/69; A61P 37/04

(52) U.S. Cl. CPC A61K 39/385; A61K 39/0208; A61K 39/05; A61K 39/07; A61K 39/08; A61K 39/09; A61K 39/092; A61K 39/215; A61K 47/6921; A61P 37/04; A61K 2039/6031; A61K 2039/6075

(57) ABSTRACT

The present invention relates to modular nanoparticle-based compositions comprising immune response modulating molecules, which are particularly useful in prophylaxis and/or treatment of diseases and disorders wherein a specific type of immune response to an antigen is desired.

### CROSS-REFERENCE TO RELATED APPLICATIONS

This application claims priority to co-pending PCT International Application Serial No. PCT/EP2022/077310 filed Sep. 30, 2022, which claims priority to European Patent Application No. 21200186.1 filed on Sep. 30, 2021, the entire content of both of which is incorporated herein by reference.

### SEQUENCE LISTING

The instant application contains a Sequence Listing which has been submitted electronically as a file in XML format and is hereby incorporated by reference in its entirety.

Said XML format file, created on Nov. 8, 2024, is named Corrected Sequence Listing.xml and is 301,770 bytes in size.

### TECHNICAL FIELD

The present invention relates to modular nanoparticle-based compositions comprising immune response modulating molecules, which are particularly useful in prophylaxis and/or treatment of diseases and disorders wherein a specific type of immune response to an antigen is desired.

### BACKGROUND

Vaccines remain the most effective tools for preventing and controlling the spread of infectious diseases. Live-attenuated vaccines are highly immunogenic, inducing long-lived antibody responses even after a single immunization. In contrast, modern, subunit vaccines (i.e. based on a soluble protein antigen) show high safety, but reduced immunogenicity and fail to induce similar durable antibody responses in humans. Induction of a strong and long lasting immune response to pathogens as well as disease-associated antigens is thus very difficult to obtain with simple subunit vaccines.

The licensed Human papillomavirus (HPV) vaccines (Cervarix®, Gardasil®, and Gardasil 9®), however, make an important exception, since they seem to have comparable immunogenicity to live-attenuated vaccines and can induce highly potent, durable antibody responses in humans, even after a single dose (Schiller et al., 2018, Schiller et al., 2012, De Vincenzo et al., 2014). Importantly, this vaccine is formed by the self-assembly of the HPV major capsid protein into virus-like particles (VLPs), which is believed to be the cause of its high potency.

In fact, many studies have established a strong causal link between the high immunogenicity of VLPs and their structural similarities to native viruses, and several strategies have been pursued, exploiting VLPs as scaffolds for the presentation of heterologous antigens, including self-antigens. These studies have collectively shown that multivalent, repetitive antigen display can, in fact, significantly increase the immunogenicity of an antigen and even induce long-lasting immunity.

We have previously described the development of a modular VLP-based vaccine platform using a split-protein (Tag/Catcher) conjugation technology for attaching antigens in a unidirectional, multivalent and repetitive manner on the surface of VLPs (WO 2016/112921). To date, this technology represents a highly powerful and versatile vaccine platform, which is not necessarily limited to VLPs but can be applied to particles displaying an antigen, including nanoparticles.

In some instances it can be desirable to modulate the ability of an antigen to induce a specific type of immune response. This is true both in terms of which arm of the immune system is preferentially activated (humoral versus cellular) or the type of vaccine-induced immunoglobulins (IgG versus IgA). Finally, as every IgG subclass has a specific biological function, it may also be important to activate certain effector mechanisms by the induction of specific IgG subclasses (Fischinger et al., 2020). For example, there are indications, such as Influenza, where antibody-dependent cellular cytotoxicity (ADCC) is known to play an important role in disease prevention and where there is a well-established link between specific favorable Fc-FcyR interactions and protection. In such case, a vaccine inducing an IgG1 dominated response would seem optimal, as this subclass profile leads to increased ADCC/antibody-dependent natural killer (NK) cell degranulation (Von Holle and Moody, 2019; DiLillo et al., 2014).

Traditionally, different immunization routes and vaccine formulations, which can include different extrinsic adjuvants, have been explored to obtain specific desired vaccine-induced immune responses.

There is thus an urgent need for a vaccine that combines the high immunogenicity of multivalent particulate vaccines with the advantages associated with the ability to tailor the immune response elicited by the vaccines and/or the ability to direct the immune system toward a specific type of immune response.

### SUMMARY

The present invention provides vaccines that form self-assembling particles displaying the vaccine antigen. The nanoparticles of the invention effectively display the vaccine antigen to induce a strong antigen-specific immune response, enabling induction of a strong antigen-specific immune response after vaccination. Importantly, the vaccine of the present invention also comprises an immune response modulating moiety (IRMM) linked to said nanoparticle or vaccine antigen.

The IRMM is surprisingly able to modulate the induced antigen-specific immune response towards a specific type of immune response that may significantly improve or alter the vaccine, e.g. such as by promoting antigen tolerance, by increasing the strength of the immune response or by preferentially activating the immune effectors that are most important for protection against a specific indication. The present invention additionally provides multiple types of IRMMs that may be used together in the same nanoparticle-based vaccine to achieve synergistic modulation of the antigen-specific immune response.

Herein is provided a composition comprising:
i) a particle-forming protein fused to a first peptide tag and, optionally, to a third peptide tag, optionally by a linker;
ii) an antigen fused to a second peptide tag, optionally by a linker; and
iii) at least one immune response modulating moiety (IRMM),
   wherein
a) i), ii) and iii) are provided as one or more polypeptides or as one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond, or by affinity conjugation, optionally by a linker; or
   ii. is fused to a fourth peptide tag, optionally by a linker; and
c) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
      whereby i)-ii)-iii) form a particle displaying said antigen and said at least one IRMM.

Also provided is a composition comprising:
i) a particle-forming protein fused to an antigen, optionally by a linker, and, optionally, to a third peptide tag, optionally by a linker; and
ii) at least one immune response modulating moiety (IRMM)
   wherein
a) i) and ii) are provided as a one or more polypeptides or as one or more polynucleotides encoding i) and ii);
b) said at least one IRMM
   i) is linked to said particle-forming protein or said antigen by fusion, by an isopeptide bond, by an ester bond or by affinity conjugation, optionally by a linker; or
   ii) is fused to a fourth peptide tag, optionally by a linker; and
c) the at least one IRMM and the particle-forming protein are capable of being linked via an isopeptide bond, via an ester bond, via chemical crosslinking, or via affinity conjugation between the third and the fourth peptide tags when present,
   whereby i)-ii) form a particle displaying said antigen and said at least one IRMM.

Herein is also provided a system comprising:
i) a particle-forming protein fused to a first peptide tag and, optionally, to a third peptide tag, optionally by a linker;
ii) said antigen fused to a second peptide tag, optionally by a linker; and
iii) at least one immune response modulating moiety (IRMM),
   wherein
a) i), ii) and iii) are in the form of one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond or by affinity conjugation, optionally by a linker; or
   ii. is fused to a fourth peptide tag, optionally by a linker; and
c) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
      whereby i)-ii)-iii) form a particle displaying said antigen and said at least one IRMM.

Also provided herein is a system comprising:
i) a particle-forming protein fused to said antigen, optionally by a linker, and, optionally, to a third peptide tag, optionally by a linker; and
ii) at least one immune response modulating moiety (IRMM)
   wherein
a) i) and ii) are in the form of one or more polynucleotides encoding i) and ii); b) said at least one IRMM
   i) is linked to said particle-forming protein or said antigen by fusion, by an isopeptide bond, by an ester bond or by affinity conjugation, optionally by a linker; or
   ii) is fused to a fourth peptide tag, optionally by a linker; and
c) the at least one IRMM and the particle-forming protein are capable of being linked via an isopeptide bond, via an ester bond, via chemical crosslinking or via affinity conjugation between the third and the fourth peptide tags when present,
   whereby i)-ii) form a particle displaying said antigen and said at least one IRMM.

Also provided herein is a cell, such as a host cell, expressing:
i) a particle-forming protein fused to a first peptide tag and, optionally, to a third peptide tag, optionally by a linker;
ii) an antigen fused to a second peptide tag, optionally by a linker; and
iii) at least one immune response modulating moiety (IRMM),
   wherein
a) i), ii) and iii) are in the form of one or more polypeptides or one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond or by affinity conjugation, optionally by a linker; or
   ii. is fused to a fourth peptide tag, optionally by a linker; and
c) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
      whereby i)-ii)-iii) form a particle displaying said antigen and said at least one IRMM.

Also provided herein is a cell, such as a host cell, expressing:
i) a particle-forming protein fused to an antigen, optionally by a linker, and, optionally, to a third peptide tag, optionally by a linker; and
ii) at least one immune response modulating moiety (IRMM)
   wherein
a) i) and ii) are in the form of one or more polypeptides or one or more polynucleotides encoding i) and ii);
b) said at least one IRMM
   i) is linked to said particle-forming protein or said antigen by fusion, by an isopeptide bond, by an ester bond or by affinity conjugation, optionally by a linker; or
   ii) is fused to a fourth peptide tag, optionally by a linker; and
c) the at least one IRMM and the particle-forming protein are capable of being linked via an isopeptide bond, via an ester bond, via chemical crosslinking or via affinity conjugation between the third and the fourth peptide tags when present,
   whereby i)-ii) form a particle displaying said antigen and said at least one IRMM.

Similarly, the present disclosure also provides the system as disclosed elsewhere herein, and/or the cell or host cell as disclosed elsewhere herein, for use in a method for inducing an immune response in a subject, the method comprising the step of administering said composition, polynucleotide or system to said subject at least once.

In some aspects is thus provided a method of modulating an immune response of a subject to an antigen, the method comprising a step of administering a composition to said subject at least once, wherein the composition comprises:
i) a particle-forming protein fused to a first peptide tag and, optionally, to a third peptide tag, optionally by a linker;
ii) said antigen fused to a second peptide tag, optionally by a linker; and
iii) at least one immune response modulating moiety (IRMM),
   wherein
a) i), ii) and iii) are provided as one or more polypeptides or as one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond or by affinity conjugation, optionally by a linker; or
   ii. is fused to a fourth peptide tag, optionally by a linker; and
c) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
      whereby i)-ii)-iii) form a particle displaying said antigen and said at least one IRMM.

In some aspects is provided a method of modulating an immune response of a subject to an antigen, the method comprising a step of administering a composition to said subject at least once, wherein the composition comprises:
i) a particle-forming protein fused to said antigen, optionally by a linker, and, optionally, to a third peptide tag, optionally by a linker; and
ii) at least one immune response modulating moiety (IRMM)
   wherein
a) i) and ii) are provided as a one or more polypeptides or as one or more polynucleotides encoding i) and ii);
b) said at least one IRMM
   i) is linked to said particle-forming protein or said antigen by fusion, by an isopeptide bond, by an ester bond or by affinity conjugation, optionally by a linker; or
   ii) is fused to a fourth peptide tag, optionally by a linker; and
c) the at least one IRMM and the particle-forming protein are capable of being linked via an isopeptide bond, via an ester bond, via chemical crosslinking or via affinity conjugation between the third and the fourth peptide tags when present,
   whereby i)-ii) form a particle displaying said antigen and said at least one IRMM.

In some aspects of the inventions is also provided a kit of parts comprising
i) a composition as defined elsewhere herein or a system as defined elsewhere herein, and
ii) optionally, a medical instrument or other means for administering the composition, and
iii) instructions for use.

### DESCRIPTION OF DRAWINGS

FIG. 1. Effect of antigen-displaying VLPs genetically modified by two different TLR4 agonist peptides on antibody responses after vaccination. Total antigen-specific (i.e. anti-SpyCatcher (SpyC)) IgG including individual isotypes (IgG1, IgG2a, IgG2b and IgG3) is measured after immunization of Balb/C mice with 2 ug modified (i.e. genetic fusion of TLR4 agonist peptide) or unmodified (i.e. SpyC-AP205) VLPs displaying the SpyCatcher model antigen. Two different TLR4 agonist peptides were tested: SpyCatcher-ggsgs-AP205-ggsgs-RYETMSIMIKSGGKY (squares) and SpyCatcher-ggsgs-AP205-ggsgs-APPHALS (triangles). Control: SpyCatcher-ggsgs-AP205 (circles). The asterisk indicates a significant difference. (A) Total IgG response. (B) IgG1 response. (C) IgG2A response. (D) IgG2b response. (E) IgG3 response.

FIG. 2. Effect of antigen-displaying VLPs genetically modified by a CD180 agonist peptide or a C3d complement peptide on antibody responses after vaccination. Total antigen-specific (i.e. anti-SARS-CoV-2 Spike RBD)) IgG including individual isotypes (IgG1, IgG2a, IgG2b and IgG3) is measured after immunization with 2 μg modified (i.e. by genetic fusion of either CD180 agonist or C3d complement peptide) or unmodified AP205 VLPs displaying the receptor binding domain of the SARS-CoV-2 Spike protein. The antigen is conjugated to the VLP using genetically fused split-protein Tag/Catcher binding partners. Serum is obtained two week after immunization. Two different peptides were tested: aCD180-AP205-RT+MC-RBD: (CSSNKSTTGSGETTTA-GSGTAGGGSGS-AP205-GTASGGSGGS-RumtrunkD9N; squares) and RT-VLP-C3d+MC-RBD: (RumtrunkD9N-GSGTAGGGSGS-AP205-GTASGGSGGS-KFLNTAKDRNRWEEPDQQLYNVEATSYA; triangles). Control: AP205+Mc-RBD. The asterisk indicates a significant difference. (A) Total IgG response. (B) IgG1 response. (C) IgG2A response. (D) IgG2b response. (E) IgG3 response.

FIG. 3. Conjugation (by isopeptide bond) of a TLR5 agonist confers new properties to a VLP displaying a model antigen (HPV L2). Anti-HPV 2 IgG titers are shown. The asterisk indicates a significant difference.

FIG. 4. Illustrative particles displaying an antigen and an IRMM. (A) The particle-forming protein forms a particle. The first peptide tag is here fused to the particle-forming protein. The antigen is fused to a second peptide tag. The IRMM is fused to a second peptide tag. The first and the second peptide tags interact via conjugation or an isopeptide bond, resulting in the formation of a particle displaying the antigen at its surface, on which the IRMM is also presented. (B) Another illustrative embodiment. The particle-forming protein forms a particle. The first peptide tag is here fused to the particle-forming protein. The antigen is fused to the second peptide tag. The IRMM is fused to the antigen. The first and the second peptide tags interact via conjugation or an isopeptide bond, resulting in the formation of a particle displaying the antigen at its surface. The IRMM fused to the antigen is also displayed. (C) Another illustrative embodiment. The particle-forming protein forms a particle. The first peptide tag is here fused to the particle-forming protein; a third peptide tag is also fused to the particle-forming protein. The antigen is fused to a second peptide tag. The IRMM is fused to a fourth peptide tag. The first and the second peptide tags interact via conjugation or an isopeptide bond, resulting in the formation of a particle displaying the antigen at its surface. The third and the fourth peptide tags also interact via conjugation or an isopeptide bond, whereby the IRMM is also displayed on the particle. (D) Another illustrative embodiment. The particle-forming protein forms a particle. The first peptide tag is here fused to the particle-forming protein. The antigen is fused to the second peptide tag. The IRMM is also fused to the second peptide tag at a separate location to the antigen. The first and the second peptide tags interact via conjugation or an isopeptide bond, resulting in the formation of a particle displaying the antigen and IRMM at its surface.

### DETAILED DESCRIPTION

### Definitions

The term “isopeptide bond” as used herein, refers to an amide bond between a carboxyl group and an amino group at least one of which is not derived from a protein main chain or alternatively viewed is not part of the protein backbone. An isopeptide bond may form within a single protein or may occur between two peptides or a peptide and a protein. Thus, an isopeptide may form intramolecularly within a single protein or intermolecularly i.e. between two peptide/protein molecules. Typically, an isopeptide bond may occur intramolecularly between two reactive amino acids: a lysine and an asparagine or aspartate. For the process to occur the two reactive amino acids need to be in close proximity in a hydrophobic environment often including aromatic residues. Finally, the autocatalytic process may be facilitated by a catalytic aspartate or glutamate residue, which do not themselves take part in the isopeptide bond.

In the case of intermolecular isopeptide bonds, the bond typically occurs between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. Thus, an isopeptide bond may form between a lysine residue and an asparagine residue or between a lysine residue and an aspartic acid residue. Particularly, isopeptide bonds can occur between the side chain amine of lysine and carboxamide group of asparagine.

The term “open reading frame” as used herein refers to a nucleotide sequence comprising in a 5′ to 3′ direction 1) a translation initiation codon, 2) one or more codons coding for one or more gene products of interest, preferably one or more protein, and 3) a translation stop codon, whereby it is understood that 1), 2) and 3) are operably linked in frame. The open reading frame will thus consist of a multiple of 3 nucleotides (triplets).

The term “sequence variant” refers to a polypeptide and/or polynucleotide sequence with at least 70%, such as 75%, such as 80%, such as 85%, such as 90%, such as 95%, such as 96%, such as, 97%, such as 98%, such as 99%, such as 99.5%, such as 100% sequence identity to said polypeptide and/or polynucleotide sequence.

The term “antigenic variant” refers to a variant of the full length of a polypeptide or a fragment of said polypeptide, wherein the fragment comprises an epitope that is recognized by a cytotoxic T lymphocyte, helper T lymphocyte and/or B cell of the host. Said fragment may be more immunogenic and thus elicit a stronger and/or longer lasting immune response than the original polypeptide from which it is derived. Preferably, the immunogenic portion of the antigenic variant will comprise at least 30%, preferably at least 50%, especially at least 75% and in particular at least 90% (e.g. 95% or 98%) of the amino acid sequence of the reference sequence. The immunogenic portion will preferably comprise all of the epitope regions of the reference sequence. The immunogenicity of said antigenic variant may be verified by any of the known methods in the art, such as the methods described in Wadhwa et al., 2015.

A first peptide tag and a second peptide tag (or binding partner) as discussed herein refer to a pair of polypeptides which associate or are linked to one another via an isopeptide bond, preferably a spontaneous isopeptide bond, or via affinity conjugation. Similarly, a third peptide tag and a fourth peptide tag (or binding partner) as discussed herein refer to a pair of polypeptides which associate or are linked to one another via an isopeptide bond, preferably a spontaneous isopeptide bond, or via affinity conjugation. Preferably for one given pair, in the case of an isopeptide bond, one of the peptide tags comprises one of the reactive residues involved in the isopeptide bond and the other peptide tag comprises the other reactive residue involved in that isopeptide bond. In the case of affinity conjugation between a first and a second molecule, e.g. via the interaction between a biotin molecule and a streptavidin molecule, one of the peptide tags of a given pair comprises or is conjugated to the first molecule and the other of the peptide tags comprises or is conjugated to the second molecule.

The term “spontaneous” as used herein refers to a bond, in particular an isopeptide bond, which can form in a protein or between peptides or proteins (e.g. between the first peptide tag and the second peptide tag) without any other agent (e.g. an enzyme catalyst) being present and/or without chemical modification of the protein or peptide e.g. without native chemical ligation or chemical coupling. A spontaneous isopeptide bond may therefore form of its own accord in the absence of enzymes or other exogenous substances or without chemical modification. Particularly however, a spontaneous isopeptide or covalent bond may require the presence of a glutamic acid or an aspartic acid residue in one of the peptides/proteins involved in the bond to allow formation of the bond.

The term “virus-like particle” or “VLP” refers to one or several recombinantly expressed viral proteins such as viral capsid proteins, which spontaneously assemble into macromolecular particulate structures mimicking the morphology of a virus coat, but lacking infectious genetic material.

The term “particle” herein refers to a virus-like particle or to a nanoparticle, on the surface of which an antigen can be displayed as described herein. The surface may be an internal surface, i.e. facing towards the inner part of the particle, or an external surface, i.e. facing towards the surroundings of the particle.

The term “self-assembly” refers to a process in which a system of pre-existing components, under specific conditions, adopts a more organised structure through interactions between the components themselves. In the present context, self-assembly refers to the intrinsic capacity of a protein, such as a viral protein, for example a capsid protein, and/or a phage protein to self-assemble into particles, in particular virus-like particles in the absence of other viral proteins, when subjected to specific conditions. “Self-assembly” does not preclude the possibility that cellular proteins, e.g. chaperones, participate in the process of intracellular VLP or nanoparticle assembly. The self-assembly process may be sensitive and fragile and may be influenced by factors such as, but not limited to, choice of expression host, choice of expression conditions, and conditions for maturing the virus-like particles. Virus capsid proteins may be able to form VLPs on their own, or in combination with several virus capsid proteins, these optionally all being identical.

The term “orientation”, as used herein, refers to the orientation of an antigen and its spatial orientation on the surface of a particle as disclosed herein, i.e. on an internal surface or on an external surface of the particle, preferably at least on the external surface. When an antigen fused to a peptide tag is linked or associated to a protein comprising a further peptide tag as disclosed herein, one molecule of antigen can only be linked to a single particle-forming protein at unique sites in both the antigen and the protein, thus creating a uniform and/or consistent presentation of said antigen with a consistent orientation. By contrast, for example, a streptavidin homo-tetramer may crosslink several proteins on the outer surface of a biotinylated nanoparticle.

The term “regularly spaced” as used herein, refers to antigens of the present invention which forms a pattern on a surface of a VLP or nanoparticle. Such pattern may be symmetric, circle-like, and/or bouquet like pattern of antigens.

The term “treatment” refers to the remediation of a health problem. Treatment may also be preventive and/or prophylactic or reduce the risk of the occurrence of a disease and/or infection. Treatment may also be curative or ameliorate a disease and/or infection.

The term “prophylaxis” refers to the reduction of risk of the occurrence or relapse of a disease and/or infection. Prophylaxis may also refer to the prevention of the occurrence of a disease and/or infection.

The term “loop” refers to a secondary structure of a polypeptide where the polypeptide chain reverses its overall direction and may also be referred to as a turn.

The term “vaccine cocktail” refers to a mixture of antigens administered together. A vaccine cocktail may be administered as a single dose or as several doses administered over a period of time. Time intervals may be, but are not limited to, administration within the same year, month, week, day, hour and/or minute. Co-vaccination and vaccine cocktail may be used interchangeably.

The term “self-antigens” refers to endogenous antigens that have been generated within previously normal cells as a result of abnormal cell metabolism.

The present methods, compositions and systems are particularly useful for modulating an immune response of a subject to an antigen. The term “modulating” herein means controlling or tailoring the immune response. For instance, the type of immune response to an antigen can be modified by using the methods and constructs described herein. In particular, the present methods, compositions and expression constructs can allow directing an immune response towards a specific type of immune response.

### Compositions

Herein is provided a composition comprising:
i) a particle-forming protein fused to a first peptide tag and, optionally, to a third peptide tag, optionally by a linker;
ii) an antigen fused to a second peptide tag, optionally by a linker; and
iii) at least one immune response modulating moiety (IRMM),
   wherein
a) i), ii) and iii) are provided as one or more polypeptides or as one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond or by affinity conjugation, optionally by a linker; or
   ii. is fused to a fourth peptide tag, optionally by a linker; and
c) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
      whereby i)-ii)-iii) form a particle displaying said antigen and said at least one IRMM.

Also provided is a composition comprising:
i) a particle-forming protein fused to an antigen, optionally by a linker, and, optionally, to a third peptide tag, optionally by a linker; and
ii) at least one immune response modulating moiety (IRMM)
   wherein
a) i) and ii) are provided as a one or more polypeptides or as one or more polynucleotides encoding i) and ii);
b) said at least one IRMM
   i) is linked to said particle-forming protein or said antigen by fusion, by an isopeptide bond, by an ester bond or by affinity conjugation, optionally by a linker; or
   ii) is fused to a fourth peptide tag, optionally by a linker; and
c) the at least one IRMM and the particle-forming protein are capable of being linked via an isopeptide bond, via an ester bond, via chemical crosslinking or via affinity conjugation between the third and the fourth peptide tags when present,
   whereby i)-ii) form a particle displaying said antigen and said at least one IRMM.

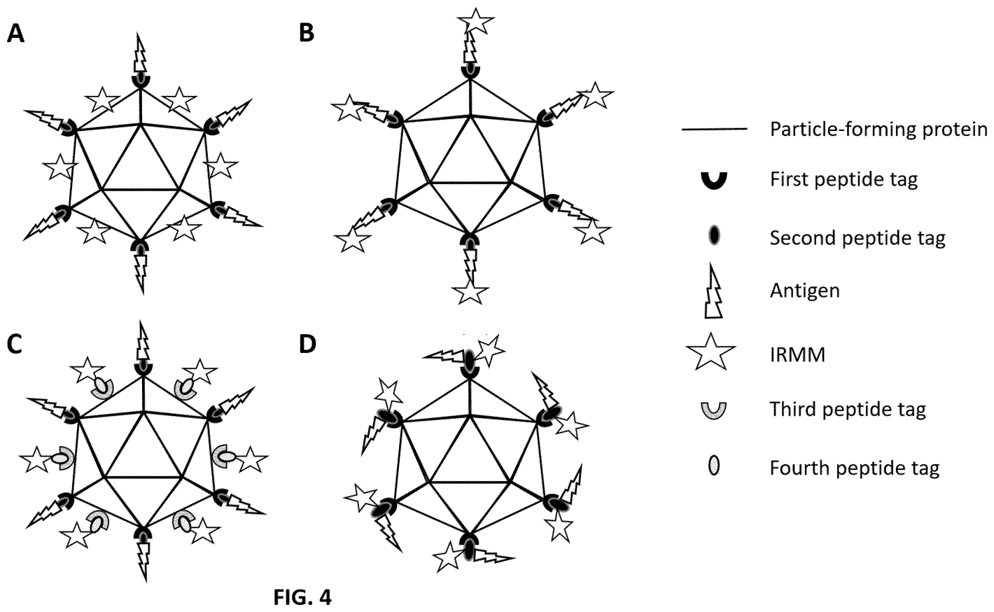

Thus, in some embodiments, the composition comprises:
i) a particle-forming protein fused to a first peptide tag, optionally by a linker;
ii) an antigen fused to a second peptide tag, optionally by a linker; and
iii) at least one immune response modulating moiety (IRMM) linked to said particle-forming protein by fusion, by an isopeptide bond or by affinity conjugation, optionally by a linker,
   wherein
a) i), ii) and iii) are provided as one or more polypeptides or as one or more polynucleotides encoding i), ii) and iii); and
b) the antigen and the particle-forming protein are capable of being linked via an isopeptide bond, via an ester bond, via chemical crosslinking or via affinity conjugation between the first and the second peptide tags,
   whereby i)-ii)-iii) form a particle displaying said antigen and said at least one IRMM. This is illustrated in FIG. 4A.

In some embodiments, the composition comprises:
i) a particle-forming protein fused to a first peptide tag, optionally by a linker; and
ii) an antigen fused to a second peptide tag and to at least one immune response modulating moiety (IRMM), optionally by separate linkers;
   wherein
a) i), and ii) are provided as one or more polypeptides or as one or more polynucleotides encoding i) and ii); and
b) the antigen and the particle-forming protein are capable of being linked via an isopeptide bond, via an ester bond, via chemical crosslinking or via affinity conjugation between the first and the second peptide tags,
   whereby i)-ii) form a particle displaying said antigen and said at least one IRMM. This is illustrated in FIG. 4B.

In some embodiments, the composition comprises:
i) a particle-forming protein fused to a first peptide tag, optionally by a linker; and
ii) an antigen and at least one immune response modulating moiety (IRMM) fused to a second peptide tag, optionally by separate linkers;
   wherein
a) i), and ii) are provided as one or more polypeptides or as one or more polynucleotides encoding i) and ii); and
b) the antigen and the particle-forming protein are capable of being linked via an isopeptide bond, via an ester bond, via chemical crosslinking or via affinity conjugation between the first and the second peptide tags,
   whereby i)-ii) form a particle displaying said antigen and said at least one IRMM.

In some embodiments, the composition comprises:
i) a particle-forming protein fused to a first peptide tag and to a third peptide tag, optionally by separate linkers;
ii) an antigen fused to a second peptide tag, optionally by a linker; and
iii) at least one immune response modulating moiety (IRMM) fused to a fourth peptide tag, optionally by a linker,
   wherein
a) i), ii) and iii) are provided as one or more polypeptides or as one or more polynucleotides encoding i), ii) and iii); and
b) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags,
      whereby i)-ii)-iii) form a particle displaying said antigen and said at least one IRMM. This is illustrated in FIG. 4C.

In some embodiments, the antigen and the particle-forming protein are capable of being linked via an isopeptide bond, via an ester bond, via chemical crosslinking or via affinity conjugation between the first and the second peptide tags upon expression, production or introduction in a cell, or via in vitro coupling.

In some embodiments, the at least one IRMM and the particle-forming protein are capable of being linked via an isopeptide bond, via an ester bond, via chemical crosslinking or via affinity conjugation between the third and the fourth peptide tags upon expression or introduction in a cell, or via in vitro coupling.

The IRMM and the particle-forming protein, first peptide tag, second peptide tag or antigen; or the antigen and the particle-forming protein may be directly or indirectly linked. In some embodiments, the IRMM and the particle-forming protein, first peptide tag, second peptide tag or antigen are indirectly linked, such as via a spacer or linker. In some embodiments, the antigen and the particle-forming protein are indirectly linked, such as via a spacer or linker.

In some embodiments, the IRMM and the particle-forming protein, or the antigen and the particle-forming protein, are linked via sortase-mediated ligation. Useful amino acid sequences for sortase-mediated ligation are further described in Tang et al., 2016 and Patterson et al., 2017.

Said compositions are useful for prophylaxis and/or treatment of a disease or a disorder, such as those described herein below.

The first, second, third and/or fourth peptide tag, the antigen, the particle-forming protein, and/or the IRMM may be encoded in one or more polynucleotides.

In some embodiments, i), ii) and iii) are provided as one or more polynucleotides encoding i), ii) and iii), wherein said particle-forming protein fused to said first peptide tag, and optionally to said third peptide tag, is encoded by a first polynucleotide.

In some embodiments, i), ii) and iii) are provided as one or more polynucleotides encoding i), ii) and iii), wherein said antigen fused to said second peptide tag is encoded by a second polynucleotide.

In some embodiments, i), ii) and iii) are provided as one or more polynucleotides encoding i), ii) and iii), wherein said at least one IRMM fused to a fourth peptide tag is encoded by a third polynucleotide.

In some embodiments, i), ii) and iii) are provided as one or more polynucleotides encoding i), ii) and iii), wherein said first and/or second polynucleotide further encodes said at least one IRMM.

The first peptide tag and the second peptide tag may be selected from peptides having the intrinsic ability to form an isopeptide bond, thereby binding to one another. Likewise, the third peptide tag and the fourth peptide tag may be selected from peptides having the intrinsic ability to form an isopeptide bond, thereby binding to one another.

The particle-forming protein has the ability to form, preferably spontaneously, a particle, such as a nanoparticle or a virus-like particle (VLP). Thus, in some embodiments, the particle is a virus-like particle. In some embodiments, the particle is a nanoparticle.

The assembled particles may closely resemble viruses or other pathogenic organisms that are recognized by the immune system, but are non-infectious because they contain no pathogenic genetic material. Due to the spontaneous formation of an isopeptide bond or due to affinity conjugation between the first peptide tag and the second peptide tag, and optionally between the third and fourth peptide tags, particles are formed, which display the antigen and the immune response modulating moiety (IRMM) on their surface, preferably on their external surface.

In some embodiments, the particle displays the antigen and the IRMM on an internal or on an external surface, preferably the particle displays the antigen and the IRMM on an external surface.

Importantly, besides consistent antigen orientation, such nanoparticles and VLPs unexpectedly have unusually beneficial antigen display characteristics including consistent antigen orientation, high-density, and regular spacing. Additionally, the IRMM is able to further stimulate the immune response or to direct the immune response towards a desirable humoral or cellular response profile. The obtained nanoparticles and VLPs can thus induce strong and/or altered humoral responses and overcome B cell tolerance. Such nanoparticles or VLPs show an increased efficiency of antigen coupling compared to chemical coupling methods, and allow antigen display with consistent antigen orientation, high-density, and regular spacing, for both large and small antigens.

The first, second, and third polynucleotides may be DNA or RNA; preferably, the first polynucleotide, the second polynucleotide and the third polynucleotide are all DNA, or all RNA.

In some embodiments, the RNA is formulated in a lipid particle formulation.

The RNA constructs and/or the mRNA transcribed from the DNA constructs may be polycistronic. In some embodiments, the first, the second and the third polynucleotides are encoded on the same ribonucleic acid molecule. In some embodiments, the first, the second and the third polynucleotides lie within the same open reading frame, whereby only one promoter sequence is needed to transcribe all the polynucleotides. In some embodiments, the first, the second and the third polynucleotides lie within separate open reading frames and may thus be regulated by separate promoters.

### Particle-Forming Proteins

The particle-forming protein may be a viral capsid protein or a viral envelope protein such as a glycoprotein. In some embodiments, the particle-forming protein is from a mammalian virus, for example a human virus.

In some embodiments, the particle-forming protein is a particle-forming protein from a hepatitis virus such as hepatitis B or E, for example a core protein from hepatitis B virus. In some embodiments, the particle-forming protein is a particle-forming protein from a norovirus such as NoV. In some embodiments, the particle-forming protein is a particle-forming protein from a papilloma virus such as Human Papilloma Virus (HPV), preferably HPV16 or HPV18, such as HPV L1. In some embodiments, the particle-forming protein a particle-forming protein from a polyomavirus such as polyomavirus vp1 (PyV). In some embodiments, the particle-forming protein is a particle-forming protein from a calicivirus such as feline calicivirus (FCV), preferably FCV VP1. In some embodiments, the particle-forming protein is a particle-forming protein from a circovirus such as a porcine circovirus (PCV), preferably PCV2 ORF2. In some embodiments, the particle-forming protein is a particle-forming protein from a nervous necrosis virus (NNV), such as NNV coat protein. In some embodiments, the particle-forming protein is a particle-forming protein from a parvovirus such as canine parvovirus (CVP), preferably CPV VP2, goose parvovirus (GPV) or porcine parvovirus (PPV), preferably structural proteins from GPV or PPV, or parvovirus B19. In some embodiments, the particle-forming protein is a particle-forming protein from a protoparvovirus such as an enteritis virus, for example mink enteritis virus (MEV), preferably MEV VP2, or duck plague virus (DPV), preferably a DPV structural protein, or a viral Gag protein, such as a HIV Gag.

The particle-forming protein may be a particle-forming protein from a plant virus, such as a cowpea virus, a tobacco virus, a tomato virus, a cucumber virus or a potato virus. In some embodiments, the plant virus is a mosaic virus, preferably Cowpea mosaic virus (CPMV). In some embodiments, the plant virus is a tobacco mosaic virus (TMV). In some embodiments, the plant virus is a tomato spotted wilt virus (TSWV). In some embodiments, the plant virus is a tomato yellow leaf curl virus (TYLCV). In some embodiments, the plant virus is a cucumber mosaic virus (CMV). In some embodiments, the plant virus is a potato virus Y (PVY).

In some embodiments, the particle-forming protein is a bacteriophage protein, such as a particle-forming protein from Salmonella virus P22, MS2, QBeta, PRR1, PP7, bacteriophage R17, bacteriophage fr, bacteriophage GA, bacteriophage SP, bacteriophage M11, bacteriophage MX1, bacteriophage NL95, bacteriophage f2 or Cb5, or such as GA-879, NF-391, ESE002, or AP205. Additional relevant bacteriophage proteins are described in Lieknina et al., 2019 and Lieknina et al., 2020.

In some embodiments, the particle-forming protein is a particle-forming protein from a virus which is common in mammals, in particular in humans. Without being bound by theory, such viruses may be better suited for expression in mammalian cells, in particular human cells, than viruses commonly found in non-mammalian organisms. Thus, in some embodiments, the virus is a hepatitis virus, such as a hepatitis B or E virus. In some embodiments, the virus is a norovirus. In some embodiments, the virus is a papilloma virus, such as Human Papilloma Virus (HPV), preferably HPV16 or HPV18. In some embodiments, the virus is a polyomavirus. In some embodiments, the virus is a parvovirus.

In some embodiments, the particle-forming protein is ferritin, i301, replicase polyprotein 1a (pp1a) or a lumazine synthase. In some embodiments, the particle-forming protein is an encapsulin, 2-oxo acid dehydrogenase subunit E2 or a 2EOZ. Other particle-forming proteins are known in the art and may also be used.

### First, Second, Third and Fourth Peptide Tags

Peptide pairs, consisting of a first peptide tag and a second peptide tag, or a third peptide tag and a fourth peptide tag, which are capable of binding to one another via the (spontaneous) formation of an isopeptide bond, are known in the art, or can be designed or obtained by methods known in the art, in particular as described in Zakeri et al., 2012, in Zakeri et al., 2010, and in application PCT/EP2021/062113.

The term “peptide tag” as used herein generally refers to a small peptide fragment which may be designed or derived directly from a particle-forming protein which naturally forms an intramolecular isopeptide bond. Peptide tags may also be identified by using a known binding partner, for example derived from a particle-forming protein naturally forming an intramolecular isopeptide bond, to screen a peptide library. The candidate peptide tags may thus be from a library, e.g. a peptide library, which can be screened for candidate peptide tags. They may also be designed in silico.

A peptide pair as understood herein thus consists of two peptide tags which can interact via the spontaneous formation of an isopeptide bond. Generally, these are also called a “tag and catcher” system, where the longer of the two peptide tags is termed “catcher” while the shorter of the two peptide tags is termed “tag”. For instance, the SpyTag/SpyCatcher system consists of a first peptide tag (SpyTag) and a second peptide tag (SpyCatcher).

In some embodiments, the first and the second peptide tags form a first “tag and catcher” system or a first peptide pair, while the third and fourth peptide tags form a second “tag and catcher” system or a second peptide pair. In preferred embodiments, the catcher and tag of the first peptide pair are different from the catcher and tag of the second peptide pair.

The “tag” may be between 5-50 amino acids in length e.g. from 10, 20, 30, 40 to 50 amino acids in length and may bind covalently via an isopeptide bond to a binding partner as defined herein. Thus, the “tag” may comprise one reactive residue involved in an isopeptide bond in the isopeptide protein used to design the binding partner (and the binding partner may comprise the other reactive residue involved in that bond), as described herein.

In some embodiments, the “tag” has a length between 7 and 47 amino acids, such as between 8 and 46 amino acids, such as between 9 and 45 amino acids, such as between 10 and 44 amino acids, such as between 11 and 43 amino acids, such as between 12 and 42 amino acids, such as between 13 and 41 amino acids, such as between 14 and 40 amino acids, such as between 15 and 39 amino acids, such as between 16 and 38 amino acids, such as between 17 and 37 amino acids, such as between 18 and 36 amino acids, such as between 19 and 35 amino acids, such as between 20 and 34 amino acids, such as between 21 and 33 amino acids, such as between 22 and 32 amino acids, such as between 23 and 31 amino acids, such as between 24 and 30 amino acids, such as between 25 and 29 amino acids, such as between 26 and 28 amino acids, such as 27 amino acids. In some embodiments, the “tag” has a length of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46 amino acids.

In some embodiments, the “catcher” is at least 20 amino acids in length. Preferably, the “catcher” has a length of 5 amino acids or more, such as 10 amino acids or more, such as 15 amino acids or more, such as 20 amino acids or more, such as 25 amino acids, such as 30 amino acids, such as 35 amino acids, such as 40 amino acids, such as 45 amino acids, such as 50 amino acids, such as 60, 70, 80, 90, 100, 125, 150, 175, 200, 225, 250, 275, 300, 325 or 350 amino acids or more. In preferred embodiments, the “catcher” is at least 20 amino acids in length. In some embodiments, the “catcher” is between 75 to 125 amino acids in length Preferably, the “catcher” has an amino acid sequence which consists of more amino acid residues than the “tag”.

In some embodiments, one of the first, second, third and fourth peptide tags comprises or is selected from the group consisting of a SpyTag (SEQ ID NO: 1), a SdyTag (SEQ ID NO: 2), a SnoopTag (SEQ ID NO: 3), a PhoTag (SEQ ID NO: 4), an EntTag (SEQ ID NO: 5), a KTag, a BacTag (SEQ ID NO: 15), a Bac2Tag (SEQ ID NO: 16), a Bac3Tag (SEQ ID NO: 17), a Bac4Tag (SEQ ID NO: 18), a RumTrunkTag (SEQ ID NO: 13 or SEQ ID NO: 14), a Rum7Tag (SEQ ID NO: 12), a RumTag (SEQ ID NO: 6), a Rum2Tag (SEQ ID NO: 7), a Rum3Tag (SEQ ID NO: 8), a Rum4Tag (SEQ ID NO: 9), a Rum5Tag (SEQ ID NO: 10), a Rum6Tag (SEQ ID NO: 11), a Bac5Tag (SEQ ID NO: 19) and a PsCsTag (SEQ ID NO: 161), or variants thereof in which at the most three, such as at the most two, such as at the most one amino acid residue has been modified. Nucleic acid sequences encoding said tags are as follows: SpyTag (SEQ ID NO: 35), SdyTag (SEQ ID NO: 36), SnoopTag (SEQ ID NO: 37), PhoTag (SEQ ID NO: 38), EntTag (SEQ ID NO: 39), KTag, BacTag (SEQ ID NO: 49), Bac2Tag (SEQ ID NO: 50), Bac3Tag (SEQ ID NO: 51), Bac4Tag (SEQ ID NO: 52), RumTrunkTag (SEQ ID NO: 47 or SEQ ID NO: 48), Rum7Tag (SEQ ID NO: 46), RumTag (SEQ ID NO: 40), Rum2Tag (SEQ ID NO: 41), Rum3Tag (SEQ ID NO: 42), Rum4Tag (SEQ ID NO: 43), Rum5Tag (SEQ ID NO: 44), Rum6Tag (SEQ ID NO: 45) Bac5Tag (SEQ ID NO: 46) and PsCsTag (SEQ ID NO: 162).

In some embodiments, the other of the first and second peptide tags, or the other of the third and fourth peptide tags, comprises or is selected from the group consisting of a SpyCatcher (SEQ ID NO: 55), a SdyCatcher (SEQ ID NO: 56), a SnoopCatcher (SEQ ID NO: 57) and an esther-forming split-protein pair. An example of an esther-forming split-protein pair is the fragment corresponding to amino acid residues 439-587 of cpe0147 (Uniprot B1R775) (SEQ ID NO: 34, DNA sequence: SEQ ID NO: 68) and the fragment corresponding to amino acid residues 565-587 of cpe0147 (Uniprot 1l R775) (SEQ ID NO: 20; DNA sequence: SEQ ID NO: 54), or variants thereof in which at the most ten, such as at the most nine, such as at the most eight, such as at the most seven, such as at the most six, such as at the most five, such as at the most four, such as at the most three, such as at the most two, such as at the most one amino acid residue has been modified. Other first or second peptide tags are presented in SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33, or variants thereof in which at the most ten, such as at the most nine, such as at the most eight, such as at the most seven, such as at the most six, such as at the most five, such as at the most four, such as at the most three, such as at the most two, such as at the most one amino acid residue has been modified; the corresponding DNA sequences are SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, and SEQ ID NO: 67.

In some embodiments, one of the first, second, third and fourth peptide tags is selected from the group consisting of FimP domain 3 (SEQ ID NO: 93), Streptococcal ancillary pilin Domain 2 (SEQ ID NO: 95), Streptococcal ancillary pilin Domain 3 (SEQ ID NO: 97), Major Pilin SpaD Domain 3 (SEQ ID NO: 99), Pilin subunit (SpaA) domain 2 (SEQ ID NO: 101), Pilin subunit (SpaA) domain 3 (SEQ ID NO: 103), Surface protein Spb1 domain 3 (SEQ ID NO: 105), PsCsCatcher (SEQ ID NO: 107), RgA Catcher (SEQ ID NO: 109), Major Pilin SpaD Domain 1 (SEQ ID NO: 111), QueenCatcher (SEQ ID NO: 113), MoonCake (SEQ ID NO: 115), Kat I (SEQ ID NO: 117) and Clib9 (SEQ ID NO: 119).

In some embodiments, the peptide pair comprises or consists of a SpyTag and a SpyCatcher. In some embodiments, the peptide pair comprises or consists of an SdyTag and an SdyCatcher. In some embodiments, the peptide pair comprises or consists of a SnoopTag and a SnoopCatcher.

In some embodiments, the peptide pair comprises or consists of truncated or modified versions of any of the above, i.e. further engineered peptide pairs, which however retain the ability to form an isopeptide bond.

A peptide tag may be altered, e.g. mutations or alterations may be introduced in any one, any two, any three or any four of the first, second, third or fourth peptide tag, i.e. in any one, any two, any three or any four of the tag and catcher. The peptide tag, i.e. the first, second, third or fourth peptide tag, should be able to covalently bind to a corresponding binding partner via an isopeptide bond spontaneously. In this respect, each peptide tag preferably comprises one of the reactive amino acid residues involved in the formation of an isopeptide bond in the isopeptide protein. Hence, each peptide tag comprises only one reactive residue from the isopeptide bond and does not comprise both reactive residues involved. Further, if the peptide tag is modified or mutated, the reactive residue in that fragment preferably remains unchanged. This means that when a homologue of a peptide tag is used, the homologue preferably still contains the reactive residue which was originally present in the original peptide tag. In some embodiments, however, the reactive residue in that fragment is also changed if the peptide tag is modified or mutated.

Preferably, the reactive residue present in the tag is an asparagine or an aspartate residue, which can form an isopeptide bond with the reactive residue of the binding partner or modified binding partner (the catcher), as described above. Thus, one peptide tag contains one reactive residue while the other peptide tag contains the other reactive residue, and thus no single peptide tag contain both reactive residues.

In some embodiments, both reactive residues are involved in the formation of an isopeptide bond. In some embodiments, the reactive residue of the first or third peptide tag is different than the reactive residue of the second or fourth peptide tag. Preferably, the reactive residue present in the “catcher” is a lysine residue. In some embodiments, the reactive residue present in the “catcher” is an asparagine or an aspartate residue. Preferably, the reactive residue present in the “tag” is an asparagine or an aspartate residue. In some embodiments, the reactive residue present in the “tag” is a lysine residue. These residues together may form the isopeptide bond.

Without being bound by theory, a third residue may be involved in the formation of the isopeptide bond. While not directly participating in the bond, this third residue may mediate the formation of the bond. Typically, the third residue is a glutamate residue. The modified binding partner preferably comprises this third residue. In other words, the peptide tag, i.e. any of the first, second, third or fourth peptide tag, preferably does not comprise this third residue, which is instead present in the modified binding partner.

Consequently, as long as the peptide pair retains the ability to form an isopeptide bond, the peptide pair may comprise or consists of truncated or modified versions of any of the above mentioned peptide tags.

Thus, in some embodiments, one of the first, second, third and fourth peptide tags is selected from the group consisting of a SpyTag (SEQ ID NO: 1), a SdyTag (SEQ ID NO: 2), a SnoopTag (SEQ ID NO: 3), a PhoTag (SEQ ID NO: 4), an EntTag (SEQ ID NO: 5), a KTag (SEQ ID NO: 163), a BacTag (SEQ ID NO: 15), a Bac2Tag (SEQ ID NO: 16), a Bac3Tag (SEQ ID NO: 17), a Bac4Tag (SEQ ID NO: 18), a RumTrunkTag (SEQ ID NO: 13 or SEQ ID NO: 14), a Rum7Tag (SEQ ID NO: 12), a RumTag (SEQ ID NO: 6), a Rum2Tag (SEQ ID NO: 7), a Rum3Tag (SEQ ID NO: 8), a Rum4Tag (SEQ ID NO: 9), a Rum5Tag (SEQ ID NO: 10), a Rum6Tag (SEQ ID NO: 11), a Bac5Tag (SEQ ID NO: 19), a PsCsTag (SEQ ID NO: 161) and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of one of the first, second, third and fourth peptide tags may be selected from the group consisting of a SpyTag (SEQ ID NO: 35), SdyTag (SEQ ID NO: 36), SnoopTag (SEQ ID NO: 37), PhoTag (SEQ ID NO: 38), EntTag (SEQ ID NO: 39), KTag (SEQ ID NO: 164), BacTag (SEQ ID NO: 49), Bac2Tag (SEQ ID NO: 50), Bac3Tag (SEQ ID NO: 51), Bac4Tag (SEQ ID NO: 52), RumTrunkTag (SEQ ID NO: 47 or SEQ ID NO: 48), Rum7Tag (SEQ ID NO: 46), RumTag (SEQ ID NO: 40), Rum2Tag (SEQ ID NO: 41), Rum3Tag (SEQ ID NO: 42), Rum4Tag (SEQ ID NO: 43), Rum5Tag (SEQ ID NO: 44), Rum6Tag (SEQ ID NO: 45), Bac5Tag (SEQ ID NO: 46), PsCsTag (SEQ ID NO: 162) and variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the other of the first and second peptide tags, or the other of the third and fourth peptide tags, is selected from the group consisting of a SpyCatcher (SEQ ID NO: 21), a SdyCatcher (SEQ ID NO: 22), a SnoopCatcher (SEQ ID NO: 23), an esther-forming split-protein pair (such as the fragment corresponding to amino acid residues 439-587 of cpe0147 (Uniprot B1R775) (SEQ ID NO: 34, DNA sequence: SEQ ID NO: 68) and the fragment corresponding to amino acid residues 565-587 of cpe0147 (Uniprot B1R775) (SEQ ID NO: 20; DNA sequence: SEQ ID NO: 54)), SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33 and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the other of the first and second peptide tags, or the other of the third and fourth peptide tags may be selected from the group consisting of the DNA sequences of SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67 and variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, one of the first, second, third and fourth peptide tags is selected from the group consisting of FimP domain 3 (SEQ ID NO: 93), Streptococcal ancillary pilin Domain 2 (SEQ ID NO: 95), Streptococcal ancillary pilin Domain 3 (SEQ ID NO: 97), Major Pilin SpaD Domain 3 (SEQ ID NO: 99), Pilin subunit (SpaA) domain 2 (SEQ ID NO: 101), Pilin subunit (SpaA) domain 3 (SEQ ID NO: 103), Surface protein Spb1 domain 3 (SEQ ID NO: 105), PsCsCatcher (SEQ ID NO: 107), RgA Catcher (SEQ ID NO: 109), Major Pilin SpaD Domain 1 (SEQ ID NO: 111), QueenCatcher (SEQ ID NO: 113), MoonCake (SEQ ID NO: 115), Kat I (SEQ ID NO: 117), Clib9 (SEQ ID NO: 119) and homologues thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of one of the first, second, third and fourth peptide tags may be selected from the group consisting of FimP domain 3 (SEQ ID NO: 94), Streptococcal ancillary pilin Domain 2 (SEQ ID NO: 96), Streptococcal ancillary pilin Domain 3 (SEQ ID NO: 98), Major Pilin SpaD Domain 3 (SEQ ID NO: 100), Pilin subunit (SpaA) domain 2 (SEQ ID NO: 102), Pilin subunit (SpaA) domain 3 (SEQ ID NO: 104), Surface protein Spb1 domain 3 (SEQ ID NO: 106), PsCsCatcher (SEQ ID NO: 108), RgA Catcher (SEQ ID NO: 110), Major Pilin SpaD Domain 1 (SEQ ID NO: 112), QueenCatcher (SEQ ID NO: 114), MoonCake (SEQ ID NO: 116), Kat I (SEQ ID NO: 118), Clib9 (SEQ ID NO: 120) and variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of a SpyTag (SEQ ID NO: 1) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a SpyCatcher (SEQ ID NO: 21) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of a SpyTag (SEQ ID NO: 35) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and a SpyCatcher (SEQ ID NO: 55) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of an SdyTag (SEQ ID NO: 2) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and an SdyCatcher (SEQ ID NO: 22) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of an SdyTag (SEQ ID NO: 36) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and an SdyCatcher (SEQ ID NO: 56) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of a SnoopTag (SEQ ID NO: 3) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a SnoopCatcher (SEQ ID NO: 23) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of a SnoopTag (SEQ ID NO: 37) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and a SnoopCatcher (SEQ ID NO: 57) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of a PsCsCatcher (SEQ ID NO: 31) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a PsCsTag (SEQ ID NO: 161) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of a PsCsCatcher (SEQ ID NO: 65) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and a PsCsTag (SEQ ID NO: 162) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of a Rumtrunk D9N (SEQ ID NO: 13) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a MoonCake (SEQ ID NO: 115) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of a Rumtrunk D9N (SEQ ID NO: 47) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and a MoonCake (SEQ ID NO: 116) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of a Rumtrunk D9N (SEQ ID NO: 13) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a Kat I (SEQ ID NO: 117) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of a Rumtrunk D9N (SEQ ID NO: 47) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and a Kat I (SEQ ID NO: 118) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of a RumTag (SEQ ID NO: 6) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a MoonCake (SEQ ID NO: 115) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of a RumTag (SEQ ID NO: 40) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and a MoonCake (SEQ ID NO: 116) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the peptide pair comprises or consists of a RumTag (SEQ ID NO: 6) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a Kat I (SEQ ID NO: 117) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid sequence of the peptide pair may comprise or consist of a RumTag (SEQ ID NO: 40) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, and a Kat I (SEQ ID NO: 118) or a variant thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the composition may further comprises a SpyLigase (SEQ ID NO: 147; DNA sequence SEQ ID NO: 148), whereby peptide tags may be locked together.

In some embodiments, the first peptide tag, the second peptide tag, the third peptide tag and/or the fourth peptide tag is a first affinity tag, a second affinity tag, a third affinity tag, and/or a fourth affinity tag, respectively.

In some embodiments, one of the first and second affinity tags is a biotin and the other of the first and second affinity tags is a streptavidin, or one of the third and fourth affinity tags is a biotin, such as the biotin according to SEQ ID NO: 153, and the other of the third and fourth affinity tags is a streptavidin, such as the streptavidin according to SEQ ID NO: 155. Said biotin may also be encoded in a nucleic acid comprising the sequence according to SEQ ID NO: 154. Said streptavidin may also be encoded in a nucleic acid comprising the sequence according to SEQ ID NO: 156.

In some embodiments, one of the first and second, or one of the third and fourth, affinity tags is a first component of a two-component nanoparticle and the other of the first and second, or the other of the third and fourth, affinity tags is a second component of said two-component nanoparticle. In some embodiments one of the first and second, or one of the third and fourth, affinity tags is 153-50A (SEQ ID NO: 157) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto and the other of the first and second, or the other of the third and fourth, affinity tags is 153-50B (SEQ ID NO: 159) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. Likewise, the nucleic acid of one of the first and second, or one of the third and fourth, affinity tags may be 153-50A (SEQ ID NO: 158) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto and the nucleic acid of the other of the first and second, or the other of the third and fourth, affinity tags may be 153-50B (SEQ ID NO: 160) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

Further useful tags that may be used as the first peptide tag, the second peptide tag, the third peptide tag and/or the fourth peptide tag are disclosed in Keeble et al., 2021. Thus, in some embodiments, the peptide pair comprises or consists of a DogTag (SEQ ID NO: 165) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto, and a DogCatcher (SEQ ID NO: 166) or a homologue thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

### Fusion of Peptide or Affinity Tags to the Particle-Forming Protein and Antigen

Changing the position where the first peptide tag is fused to the particle-forming protein may allow changing the orientation of the antigen on the particle. This may be performed to enable the best possible display of the most important epitopes of the antigen. The best possible orientation may be different from antigen to antigen.

Epitopes of specific monoclonal antibodies may be mapped on the antigen structure, whereby it is possible to determine which epitopes are accessible after conjugation of the antigen to the particle. Specifically, one may measure binding between a specific monoclonal antibody and the complex of the antigen bound to the particle (antigen:particle complex), such as by using ELISA or another affinity-measuring technique (e.g. Attana), and thereby determine the orientation of the antigen. Cryo-electron microscopy may also be used to determine the structure of the entire antigen:particle complex. If the antigen contains a functional binding epitope, binding-assays may be conducted to determine if the epitope is exposed or hidden in the final antigen:particle complex.

Changing the position where the second peptide tag is fused to the antigen will allow changing the orientation of the antigen on the particle. This may be performed to enable the best possible display of the most important epitopes of the antigen. The best possible orientation may be different from antigen to antigen.

In some embodiments, the first and/or third peptide tag, or one or more first and/or second affinity tags, are fused to the N-terminus of the particle-forming protein. In other embodiments, the first and/or third peptide tag, or one or more first and/or second affinity tags, are fused to the C-terminus of the particle-forming protein. In other embodiments, the nucleic acid(s) encoding the first and/or third peptide tag, or one or more first and/or second affinity tag is inserted in-frame in the coding sequence of the particle-forming protein. The fusion protein may comprise a linker between the first and/or third peptide tag, or one or more of the first and/or second affinity tags, and the particle-forming protein.

Similarly, in some embodiments, the second peptide tag, or one or more second affinity tags, are fused to the N-terminus of the antigen. In other embodiments, the second peptide tag, or one or more second affinity tags, are fused to the C-terminus of the antigen. In other embodiments, the nucleic acid(s) encoding the second peptide tag, or one or more second affinity tags, are inserted in-frame in the coding sequence of the antigen. The fusion protein may comprise a linker between the second peptide tag, or one or more second affinity tags, and the antigen.

### Immune Response Modulating Moiety (IRMM)

The compositions as disclosed herein comprise at least one immune response modulating moiety (IRMM). The IRMM is a molecule that can modulate or alter the immune response towards the antigen presented by the particle. This altered response may be desirable, e.g. to induce tolerance towards the antigen, to stimulate a stronger antigen-specific humoral and/or cellular immune response towards the antigen and/or to alter the antigen-specific humoral and/or cellular immune response towards the antigen, such as by altering the Ig isotype ratio or Th1 vs. Th2 cell induction.

In some embodiments, the IRMM is fused at the N-terminus of the particle-forming protein, optionally by a linker. In some embodiments, the IRMM is fused at the C-terminus of the particle-forming protein, optionally by a linker. In some embodiments, the IRMM is fused within an internal region, such as in an exposed loop, of the particle-forming protein, optionally by a linker.

More than one IRMM, such as two, three or more IRMMs, may be linked to the same particle-forming protein. In some embodiments, the IRMMs are identical, i.e. identical molecules. In some embodiments, the at least one IRMM comprises at least two, such as at least three, such as at least four, such as at least five or such as at least 10 different IRMMs.

In some embodiments, the one or more IRMMs are fused, optionally by one or more linkers, to the N-terminus of the particle-forming protein, the C-terminus of the particle-forming protein, and/or within an internal region, such as in an exposed loop, of the particle-forming protein. The one or more IRMMs may also be fused to the first peptide tag and/the second peptide tag. In some embodiments, the one or more IRMMs are fused to the antigen. In some embodiments, the IRMMs are fused to at least two fusion locations selected independently from the group consisting of the N-terminus of the particle-forming protein, the C-terminus of the particle-forming protein, an internal region, such as an exposed loop, of the particle-forming protein, the first peptide tag, the second peptide tag and the antigen.

In some embodiments, a first and a second IRMMs are fused, optionally by one or more linkers, to one or two fusion locations selected from the group consisting of the N-terminus of the particle-forming protein, the C-terminus of the particle-forming protein, an internal region, such as an exposed loop, of the particle-forming protein, the first peptide tag, the second peptide tag and the antigen. In some embodiments, the first and second IRMMs are identical molecules. In some embodiments, the first and second IRMMs are different molecules.

In some embodiments a first, a second and a third IRMMs are fused, optionally by one or more linkers, to one, two or three fusion locations selected from the group consisting of the N-terminus of the particle-forming protein, the C-terminus of the particle-forming protein, an internal region, such as an exposed loop, of the particle-forming protein, the first peptide tag, the second peptide tag and the antigen. In some embodiments, the first, second and third IRMMs are identical molecules. In some embodiments, the first, second and third IRMMs are different molecules.

An IRMM may be linked to a further IRMM, such as by fusion, optionally by a linker. Thus, in some embodiments, a first IRMM is fused to a second IRMM, optionally by a linker.

The particle-forming protein may also naturally comprise an IRMM. For example, part of the particle-forming protein may have the same effect as an IRMM, e.g. as a TLR agonist. Thus, in some embodiments, the particle-forming comprises an inherent IRMM, which may be internal to the particle once formed, or external; preferably, the inherent IRMM is internalized in the particle once formed. Thus, in some embodiments, the particle-forming protein comprises an internal IRMM. In some embodiments, the particle-forming protein is lumazine synthase (SEQ ID NO: 70) or a homologue thereof having at least 60%, such as at least 70%, such as at least 80%, such as at least 90% sequence identity thereto, wherein the particle-forming protein comprises an internal IRMM, such as a TLR agonist, e.g. a TLR4 agonist.

The IRMM is able to modulate the immune cell by stimulating specific cells of the immune system, such as by binding to and stimulating receptors of specific immune cells. Thus, in some embodiments, the IRMM and/or the further IRMM is an immune cell targeting moiety.

In some embodiments, the IRMM and/or the further IRMM is capable of binding to a surface receptor of an immune cell. In some embodiments, the further IRMM is the first, second or third IRMM.

It may be useful to that the IRMM is a short peptide or a small protein as it can be genetically fused to the particle. Additionally, short peptides are too short to become B-cell targets on their own, which could limit their effect.

In some embodiments, the IRMM is selected from the group consisting of a Toll-like receptor (TLR) agonist, a dendritic cell receptor binding moiety, a mast cell receptor binding moiety, a universal T cell epitope, a Complement component 3 (C3) binding moiety, an epitope recognized by a regulatory T cell, a Dectin-1 receptor targeting moiety, a cytokine or a chemokine.

In some embodiments, the IRMM is a Toll-like receptor (TLR) agonist. In some embodiments, the TLR agonist is an agonist of TLR4, such as a peptide according to SEQ ID NO: 121 (DNA sequence: SEQ ID NO: 122). In some embodiments, the TLR agonist is an agonist of CD180, such as a peptide according to SEQ ID NO: 123 (DNA sequence: SEQ ID NO: 124). In some embodiments, the TLR agonist is an agonist of TLR5, such as a recombinant flagellin according to SEQ ID NO: 141 (DNA sequence: SEQ ID NO: 142). In some embodiments, the TLR agonist is an agonist of TLR2, such as a peptide according to SEQ ID NO: 133 (DNA sequence: SEQ ID NO: 134). Further examples of useful TLR agonists are known to the person skilled in the art, e.g. such as the bacterial protein Toll-like receptor agonists disclosed in Kumar et al., 2019.

In some embodiments, the IRMM comprises or consists of a sequence selected from the group consisting of SEQ ID NO: 121, SEQ ID NO: 123, SEQ ID NO: 141, SEQ ID NO: 133, and homologues thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto. Likewise, in some embodiments, the IRMM comprises or consists of a sequence selected from the group consisting of SEQ ID NO: 122, SEQ ID NO: 124, SEQ ID NO: 142, SEQ ID NO: 134, and variants thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the IRMM is a dendritic cell receptor binding moiety. In some embodiments, the dendritic cell receptor binding moiety is a peptide according to SEQ ID NO: 127 (DNA sequence: SEQ ID NO: 128). In some embodiments, the dendritic cell receptor binding moiety is DEC-205 (SEQ ID NO: 149; DNA sequence: SEQ ID NO: 150).

In some embodiments, the IRMM comprises or consists of a sequence selected from the group consisting of SEQ ID NO: 127, SEQ ID NO: 149, and homologues thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto. Likewise, in some embodiments, the IRMM comprises or consists of a sequence selected from the group consisting of SEQ ID NO: 128, SEQ ID NO: 149, and variants thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the IRMM is a mast cell receptor binding moiety. In some embodiments, the mast cell receptor binding moiety is a Mastoparan-7, such as a peptide according to SEQ ID NO: 129 (DNA sequence: SEQ ID NO: 130) or SEQ ID NO: 131 (DNA sequence: SEQ ID NO: 132).

In some embodiments, the IRMM comprises or consists of a sequence selected from the group consisting of SEQ ID NO: 129, SEQ ID NO: 131, and homologues thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto. Likewise, in some embodiments, the IRMM comprises or consists of a sequence selected from the group consisting of SEQ ID NO: 130, SEQ ID NO: 132, and variants thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the IRMM is a Complement component 3 (C3) binding moiety. In some embodiments, the C3 binding moiety is a peptide according to SEQ ID NO: 125 (DNA sequence: SEQ ID NO: 126).

In some embodiments, the IRMM comprises or consists of SEQ ID NO: 125 or a homologue thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto. Likewise, in some embodiments, the IRMM comprises or consists of SEQ ID NO: 126 or a variant thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the IRMM is a universal T cell epitope. In some embodiments, the IRMM is an epitope recognized by a regulatory T cell. In some embodiments, the IRMM is a Dectin-1 receptor targeting moiety. In some embodiments, the IRMM is a cytokine. In some embodiments, the IRMM is a chemokine.

In some embodiments, the IRMM is a T helper cell type 1 (Th1) response-biasing peptide. In some embodiments, the IRMM is a T helper cell type 1 (Th2) response-biasing peptide. In some embodiments, the Th2 response-biasing peptide is a synthetic Th2 response-biasing peptide, such as a peptide according to SEQ ID NO: 135 (DNA sequence: SEQ ID NO: 136), SEQ ID NO: 137 (DNA sequence: SEQ ID NO: 138) or SEQ ID NO: 139 (DNA sequence: SEQ ID NO: 140).

In some embodiments, the IRMM comprises or consists of a sequence selected from the group consisting of SEQ ID NO: 135, SEQ ID NO: 137, SEQ ID NO: 139, and homologues thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto. Likewise, in some embodiments, the IRMM comprises or consists of a sequence selected from the group consisting of SEQ ID NO: 136, SEQ ID NO: 138, SEQ ID NO: 140, and variants thereof having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

The immune response modulating moiety may also be selected from any of the peptides listed in Table 1, below.

TABLE 1
Other examples of useful IRMMs.

| Name of | Amino acid | | |
|---|---|---|---|
| peptide | sequence | Target | Effect |
| GIFN4 | mutations to IFNg | Interferon-gamma | Induces upregulation of |
| | protein | receptor | HLA-I, but not PD-L1. |
| | (138aa): A23E D24E | | (normally PD-L1 is |
| | N25K H111D K74A | | upregulated by IFNg) |
| | E75Y N83R | | |
| | (Mendoza et al., | | |
| | 2019) | | |
| CL3 | SEQ ID NO: 167 | M cells in Peyer's | Th2 immune response |
| | | Patch (Ganglioside | |
| | | GM1) | |
| Co1 | SEQ ID NO: 168 | M cells in Peyer's | Th2 immune response, |
| | | Patch | significant difference |
| | | | for IgG1 |
| CKS9 | SEQ ID NO: 169 | M cells in Peyer's | Targeting to the Peyer's |
| | | Patch | Patch |
| Peptide 3 | SEQ ID NO: 170 | specific DC-binding | Antigen specific IFNg |
| | | peptides | and TNFa production of |
| | | | CD4 T cells; peptide |
| | | | targets specifically DCs |
| | | | (no binding to |
| | | | monocytes, T cells, B |
| | | | cells, endothelial cells or |
| | | | fibroblasts) |
| Peptide 12 | SEQ ID NO: 171 | specific DC-binding | Peptide targets |
| | | peptides | specifically DCs (no |
| | | | binding to monocytes, T |
| | | | cells, B cells, endothelial |
| | | | cells or fibroblasts) |
| Peptide 18 | SEQ ID NO: 172 | specific DC-binding | Peptide targets |
| | | peptides | specifically DCs (no |
| | | | binding to monocytes, T |
| | | | cells, B cells, endothelial |
| | | | cells or fibroblasts) |
| IRMM_01 | SEQ ID NO: 173 | DCs | Peptide that specifically |
| | | | targets DCs |
| IRMM_02 | SEQ ID NO: 174 | DCs | Peptide that specifically |
| | | | targets DCs |
| p17 | SEQ ID NO: 175 | APCs, CD11c/ | Ag-specific T cell priming |
| | | CD18 expressing | and antibody production |
| | | cells | (IgG1 and IgG2b), IFNg |
| | | | production of CD8+ T |
| | | | cells |
| p18 | SEQ ID NO: 176 | APCs, CD11c/ | Ag-specific T cell priming |
| | | CD18 expressing | and antibody production |
| | | cells | |
| p30 | SEQ ID NO: 177 | APCs, CD11c/ | Ag-specific T cell priming |
| | | CD18 expressing | and antibody production |
| | | cells | |
| PCP1 | SEQ ID NO: 178 | CD40-binding | Peptide enhances |
| | | (APCs) | binding to CD40-positive |
| | | | cells |
| PCP3 | SEQ ID NO: 179 | CD40-binding | Peptide enhances |
| | | (APCs) | binding to CD40-positive |
| | | | cells |
| PCP4 | SEQ ID NO: 180 | CD40-binding | Peptide enhances |
| | | (APCs) | binding to CD40-positive |
| | | | cells |
| XS52.1 | SEQ ID NO: 181 | Langerhans cells | Peptide enhances |
| | | | binding to Langerhans |
| | | | cells |
| IRMM_03 | SEQ ID NO: 182 | Neutrophil | Cell-specific peptide |
| | | | binding by human |
| | | | neutrophils |
| IRMM_04 | SEQ ID NO: 183 | Neutrophil | Cell-specific peptide |
| | | | binding by human |
| | | | neutrophils |
| IRMM_05 | SEQ ID NO: 184 | phagocytic | The synthetic peptide is |
| | | leukocytes | a chemoattractant for |
| | | (monocytes/ | Jukat T cells |
| | | neutrophils) | |
| IRMM_06 | SEQ ID NO: 185 | phagocytic | The synthetic peptide is |
| | | leukocytes | a chemoattractant for |
| | | (monocytes/ | Jukat T cells |
| | | neutrophils) | |
| IRMM_07 | SEQ ID NO: 186 | phagocytic | Chemoattractant |
| | | leukocytes | peptides for human |
| | | (monocytes/ | leukocytes |
| | | neutrophils) | |

In some embodiments, the particle-forming protein is AP205 (SEQ ID NO: 143) and the IRMM is GIFN4. In some embodiments, the particle-forming protein is AP205 (SEQ ID NO: 143) and the IRMM is CL3 as set forth in SEQ ID NO: 167. In some embodiments, the particle-forming protein is AP205 (SEQ ID NO: 143) and the IRMM is Col as set forth in SEQ ID NO: 168. In some embodiments, the particle-forming protein is AP205 (SEQ ID NO: 143) and the IRMM is CKS9 as set forth in SEQ ID NO: 169. In some embodiments, the particle-forming protein is AP205 (SEQ ID NO: 143) and the IRMM is Peptide 3 as set forth in SEQ ID NO: 170. In some embodiments, the particle-forming protein is AP205 (SEQ ID NO: 143) and the IRMM is Peptide 12 as set forth in SEQ ID NO: 171. In some embodiments, the particle-forming protein is AP205 (SEQ ID NO: 143) and the IRMM is Peptide 18 as set forth in SEQ ID NO: 172. In some embodiments, the particle-forming protein is AP205 (SEQ ID NO: 143) and the IRMM is IRMM_01 as set forth in SEQ ID NO: 173. In some embodiments, the particle-forming protein is AP205 (SEQ ID NO: 143) and the IRMM is IRMM_02 as set forth in SEQ ID NO: 174. In some embodiments, the particle-forming protein is AP205 (SEQ ID NO: 143) and the IRMM is p17 as set forth in SEQ ID NO: 175. In some embodiments, the particle-forming protein is AP205 (SEQ ID NO: 143) and the IRMM is p18 as set forth in SEQ ID NO: 176. In some embodiments, the particle-forming protein is AP205 (SEQ ID NO: 143) and the IRMM is p30 as set forth in SEQ ID NO: 177. In some embodiments, the particle-forming protein is AP205 (SEQ ID NO: 143) and the IRMM is PCP1 as set forth in SEQ ID NO: 178. In some embodiments, the particle-forming protein is AP205 (SEQ ID NO: 143) and the IRMM is PCP3 as set forth in SEQ ID NO: 179. In some embodiments, the particle-forming protein is AP205 (SEQ ID NO: 143) and the IRMM is PCP4 as set forth in SEQ ID NO: 180. In some embodiments, the particle-forming protein is AP205 (SEQ ID NO: 143) and the IRMM is XS52.1 as set forth in SEQ ID NO: 181. In some embodiments, the particle-forming protein is AP205 (SEQ ID NO: 143) and the IRMM is IRMM_03 as set forth in SEQ ID NO: 182. In some embodiments, the particle-forming protein is AP205 (SEQ ID NO: 143) and the IRMM is IRMM_04 as set forth in SEQ ID NO: 183. In some embodiments, the particle-forming protein is AP205 (SEQ ID NO: 143) and the IRMM is IRMM_05 as set forth in SEQ ID NO: 184. In some embodiments, the particle-forming protein is AP205 (SEQ ID NO: 143) and the IRMM is IRMM_06 as set forth in SEQ ID NO: 185. In some embodiments, the particle-forming protein is AP205 (SEQ ID NO: 143) and the IRMM is IRMM_07 as set forth in SEQ ID NO: 186.

### Diseases, Medical Indications and Associated Antigens

The present invention is a novel, generic, and easy-to-use-approach to conjugate various antigens and immune response modulating moieties to a nanoparticle or VLP directly in the cell in which the nanoparticle or VLP is to be expressed, i.e. in vivo. Depending on the antigen, the present compositions can be used for prophylaxis and/or treatment of a wide range of diseases. In particular, the compositions of the present invention may be effective for induction of mucosal immune responses.

The diseases which the present invention may be used for prophylaxis and/or treatment of include, but are not limited to, cancers, cardiovascular diseases, allergic diseases, chronic diseases, neurologic diseases, infectious diseases, respiratory diseases, and/or gastrointestinal diseases. Antigens are typically peptides, polypeptides or proteins or fragments thereof, i.e. they comprise or consist of an amino acid sequence.

In some embodiments, an antigen which is associated with at least one cancer disease is linked to the particle-forming protein as described herein via the interaction between the first peptide tag and the second peptide tag. In a further embodiment the present compositions may be used for prophylaxis and/or treatment of the cancer and/or cancers which the antigen is associated with.

In some embodiments, an antigen which is associated with at least one cardiovascular disease is linked to the particle-forming protein as described herein, via the interaction between the first peptide tag and the second peptide tag. In a further embodiment the present compositions can be used for prophylaxis and/or treatment of the cardiovascular disease and/or cardiovascular diseases which the antigen is associated with.

In some embodiments, an antigen which is associated with at least one allergic disease or allergic reaction is linked to the particle-forming protein as described herein, via the interaction between the first peptide tag and the second peptide tag. In a further embodiment the present compositions can be used for prophylaxis and/or treatment of the allergic disease or allergic reaction and/or allergic diseases or reactions which the antigen is associated with.

In some embodiments, an antigen which is associated with at least one infectious disease is linked to the particle-forming protein as described herein, via the interaction between the first peptide tag and the second peptide tag. In a further embodiment the present compositions can be used for prophylaxis and/or treatment of the infectious disease and/or infectious diseases which the antigen is associated with.

In some embodiments, an antigen which is associated with at least one chronic disease is linked to the particle-forming protein as described herein, via the interaction between the first peptide tag and the second peptide tag. In a further embodiment the present compositions can be used for prophylaxis and/or treatment of the chronic disease and/or chronic diseases which the antigen is associated with.

In some embodiments, an antigen which is associated with at least one neurologic disease is linked to the particle-forming protein as described herein, via the interaction between the first peptide tag and the second peptide tag. In a further embodiment the present compositions can be used for prophylaxis and/or treatment of the neurologic disease and/or neurologic diseases which the antigen is associated with.

In some embodiments, an antigen which is associated with at least one respiratory disease is linked to the particle-forming protein as described herein, via the interaction between the first peptide tag and the second peptide tag. In a further embodiment the present compositions can be used for prophylaxis and/or treatment of the respiratory disease and/or respiratory diseases which the antigen is associated with.

In some embodiments, an antigen which is associated with at least one gastrointestinal disease is linked to the particle-forming protein as described herein, via the interaction between the first peptide tag and the second peptide tag. In a further embodiment the present compositions can be used for prophylaxis and/or treatment of the gastrointestinal disease and/or gastrointestinal diseases which the antigen is associated with.

In some embodiments, an antigen which is associated with at least one lipid disorder is linked to the particle-forming protein as described herein, via the interaction between the first peptide tag and the second peptide tag. In a further embodiment the present compositions can be used for prophylaxis and/or treatment of the lipid disorder and/or lipid disorders which the antigen is associated with.

In some embodiments, an antigen which is associated with at least one immune-inflammatory disease is linked to the particle-forming protein as described herein, via the interaction between the first peptide tag and the second peptide tag. In a further embodiment the present compositions can be used for prophylaxis and/or treatment of the immune-inflammatory disease and/or immune-inflammatory diseases which the antigen is associated with.

In some embodiments, an antigen which is associated with at least one viral disease is linked to the protein, such as a particle-forming protein as described herein, via the interaction between the first peptide tag and the second peptide tag. In a further embodiment the present compositions can be used for prophylaxis and/or treatment of the viral disease which the antigen is associated with. In some embodiments, the viral disease is caused by a coronavirus such as SARS-CoV-2, malaria, tuberculosis, HIV or influenza.

A non-exhaustive list of antigens which may be used with the present invention is outlined in Table 2 and Table 3. In addition, Table 3 shows examples of specific diseases the antigens are associated with as well as examples of patient groups which may be in need of prophylaxis and/or treatment using the present compositions.

Relevant antigens include: hemagglutinin, GD2, EGF-R, CEA, CD52, CD21, neuraminidase, human melanoma protein gp100, human melanoma protein melan-A/MART1, HIV envelope protein, M2e, VAR2CSA, ICAM1, CSP, Dengue virus NS1, Dengue virus envelope protein, Chikungunya virus envelope protein, tyrosinase, HCV E2, NA17-A nt, MAGE-3, HPV 16 E7, HPV L2, PD1, PD-L1, CTLA-4, p53, hCG, Fel d1, EGRFvIII, endoglin, ANGPTL-3, CSPG4, CTLA-4, HER2, IgE, IL-1 beta, IL-5, IL-13, IL-17, IL-22, IL-31, IL-33, TSLP, NGF and (IHNV) G-protein, a lymphotoxin such as lymphotoxin α or β, a lymphotoxin receptor, a receptor activator of nuclear factor kB ligand, vascular endothelial growth factor VEGF, a VEGF receptor, IL-23 p19, ghrelin, CCL21, CXCL12, SDF-1, M-CSF, MCP-1, endoglin, GnRH, TRH, eotaxin, bradykinin, BLC, TNF-α, amyloid β peptide A, angiotensin, gastrin, progastrin, CETP, CCR5, C5a, CXCR4, Des-Arg-bradykinin, GnRH peptide, angiotensin peptide or TNF peptide.

In some embodiments, the antigen is an antigenic fragment or antigenic variant of hemagglutinin, GD2, EGF, EGF-R, CEA, CD52, CD21, neuraminidase, human melanoma protein gp100, human melanoma protein melan-A/MART1, HIV envelope protein, M2e, VAR2CSA, ICAM1, CSP, Dengue virus NS1, Dengue virus envelope protein, Chikungunya virus envelope protein, tyrosinase, HCV E2, NA17-A nt, MAGE-3, HPV 16 E7, HPV L2, PD1, PD-L1, CTLA-4, p53, hCG, Fel d1, EGRFvIII, endoglin, ANGPTL-3, CSPG4, CTLA-4, HER2, IgE, IL-1 beta, IL-5, IL-13, IL-17, IL-22, IL-31, IL-33, TSLP, NGF and (IHNV) G-protein, a lymphotoxin such as lymphotoxin α or β, a lymphotoxin receptor, a receptor activator of nuclear factor kB ligand, vascular endothelial growth factor VEGF, a VEGF receptor, IL-23 p19, ghrelin, CCL21, CXCL12, SDF-1, M-CSF, MCP-1, endoglin, GnRH, TRH, eotaxin, bradykinin, BLC, TNF-α, amyloid β peptide A, angiotensin, gastrin, progastrin, CETP, CCR5, C5a, CXCR4, Des-Arg-bradykinin, GnRH peptide, angiotensin peptide or TNF peptide. This antigenic fragment or antigenic variant may be better at inducing a stronger immune response than the corresponding antigen. Thus, in some embodiments, the protein sequence of the antigenic fragment or antigenic variant is a homologue of the corresponding antigen, having at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, the antigenic fragment or antigenic variant is encoded by a polypeptide. Said polypeptide may consist or comprise of a nucleic acid sequence variant of the corresponding natural antigen, the nucleic acid sequence variant having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity to the corresponding antigen.

It is desirable that the cell upon transfection and expression of the antigen and the particle-forming protein, each fused to a tag as described herein, can form self-assembling particles displaying the antigen linked to the particle-forming protein. This may be assessed by relevant methods as known to the person skilled in the art.

The compositions of the present invention may as well be used against other diseases and/or use other antigens than the ones listed herein.

In some embodiments of the present invention, the medical indication is selected from the group consisting of a cardiovascular disease, an immune-inflammatory disease, a respiratory disease, a gastrointestinal disease, a chronic disease, a neurologic disease, an infectious disease, a lipid disorder and cancer. In a particular embodiment, the medical indication is an immune-inflammatory disease. In another particular embodiment, the medical indication is a cardiovascular disease. In another embodiment the medical indication is a chronic disease. In another embodiment the medical indication is a neurologic disease. In another embodiment, the medical indication is an infectious disease.

In another embodiment, the medical indication is cancer. In yet another embodiment, the medical indication is a respiratory disease. In another embodiment, the medical indication is a gastrointestinal disease. In another embodiment, the medical indication is a lipid disorder.

In some embodiments, the antigen is a polypeptide, peptide and/or an antigenic fragment of a polypeptide associated with an abnormal physiological response, such as a cardiovascular disease and/or an allergic reaction/disease. In a particular embodiment the abnormal physiological response is a cancer.

In a further embodiment the antigen is a protein, peptide and/or an antigenic fragment associated with a medical indication as disclosed herein.

TABLE 2
Non-exhaustive list of antigens or parts hereof that could be used in treatment
of specific diseases/medical indications in various patient groups.

| Examples of | Examples of a | Examples of |
|---|---|---|
| antigens | specific disease | patient group |
| (non-exhaustive) | (non-exhaustive) | (non-exhaustive) |
| Her2/Neu | Breast cancer | Females overexpressing Her2 |
| (ERBB2) | | |
| Her2/Neu | Gastric cancer | Males and females overexpressing Her2 |
| (ERBB2) | | |
| Her2/Neu | Ovarian cancer | Females overexpressing Her2 |
| (ERBB2) | | |
| Her2/Neu | Uterine serous | Postmenopausal Females |
| (ERBB2) | carcinoma | overexpressing Her2 |
| Survivin | Cancer types | Males and non-pregnant females |
| | overexpressing Survivin | overexpressing Survivin |
| PCSK9 | Cardiovascular disease | Males and females with dyslipidemia |
| PCSK9 | Cardiovascular disease | Males and females with atherosclerosis |
| PCSK9 | Cardiovascular disease | Males and females with |
| | | hypercholesterolemia |
| ANGPTL3 | Cardiovascular disease | Males and females with dyslipidemia |
| ANGPTL3 | Cardiovascular disease | Males and females with atherosclerosis |
| ANGPTL3 | Cardiovascular disease | Males and females with |
| | | hypercholesterolemia |
| Interleukin-5 | Asthma | Males and females with eosinophilia |
| Interleukin-5 | nasal polyposis | Males and females with eosinophilia |
| Interleukin-5 | atopic dermatitis | Males and females with eosinophilia |
| Interleukin-5 | eosinophilic esophagitis | Males and females with eosinophilia |
| Interleukin-5 | Hypereosinophilic | Males and females with eosinophilia |
| | syndrome | |
| Interleukin-5 | Churg-Strauss | Males and females with eosinophilia |
| | syndrome | |
| Ag85A | Tuberculosis | Males and females with tuberculosis |
| PfRH5 | Malaria | Males and females with malaria |
| VAR2CSA | Malaria | Females with malaria |
| PfEMP1, CIDR1a | Malaria | Males and females with malaria |
| GLURP | Malaria | Males and females with malaria |
| MSP3 | Malaria | Males and females with malaria |
| Pfs25 | Malaria | Males and females with malaria |
| CSP | Malaria | Males and females with malaria |
| PfSEA-1 | Malaria | Males and females with malaria |
| Hemagglutinin HA | Influenza | Males and females with influenza |
| Interleukin-17 | Psoriasis | Males and females with Psoriasis |
| Interleukin-17 | Multiple sclerosis | Males and females with multiple |
| | | sclerosis |
| Interleukin-17 | Rheumatoid arthritis | Males and females with rheumatoid |
| | | arthritis |
| Interleukin-17 | Inflammatory bowel | Males and females with inflammatory |
| | diseases | bowel diseases |
| Interleukin-17 | Asthma | Males and females with Asthma |
| IL-33 | Asthma | Males and females with Asthma |
| IgE | Asthma | Males and females with Asthma |
| Gp160 | HIV | Males and females with HIV |
| Gp120 | HIV | Males and females with HIV |
| Gp40 | HIV | Males and females with HIV |
| GD2 | Cancer cell types | Males and females with GD2 expressing |
| | expressing GD2 (e.g. | tumors. |
| | melanomas, | |
| | osteosarcoma and soft- | |
| | tissue sarcomas) | |
| EGF-R | Cancer cell types | Males and females with EGF-R |
| | expressing EGF-R (e.g. | expressing tumors. |
| | metastatic colorectal | |
| | and head and neck | |
| | cancer) | |
| CEA | Cancer cell types | Males and females with CEA expressing |
| | expressing CEA (e.g. | tumors. |
| | colon and rectal cancer | |
| | or pancreatic, breast, | |
| | ovary, or lung cancer. | |
| CD52 | Chronic lymphocytic | Males and females with chronic |
| | leukemia | lymphocytic leukemia (CLL), cutaneous |
| | (CLL), cutaneous T-cell | T-cell lymphoma (CTCL) and T-cell |
| | lymphoma (CTCL) and | lymphoma or multiple sclerosis. |
| | T-cell lymphoma or | |
| | multiple sclerosis | |
| CD21 | B-cell cancers | Males and females with B-cell cancers |
| human | Cancer cell types | Males and females with melanoma. |
| melanoma | expressing human | |
| protein gp100 | melanoma protein gp | |
| | 100 (e.g. Melanoma). | |
| human | Cancer cell types | Males and females with melanoma. |
| melanoma | expressing human | |
| protein melan- | melanoma protein | |
| A/MART-1 | melan-A/MART- | |
| | 1M(e.g. elanoma) | |
| tyrosinase | Melanoma | Males and females with melanoma |
| NA 17-A nt | Melanoma | Males and females with melanoma |
| protein | | |
| MAGE-3 protein | melanoma, non-small | Males and females with melanoma, non- |
| | cell lung cancer, | small cell lung cancer or hematologic |
| | hematologic | malignancies |
| | malignancies. | |
| p53 protein | Cancer cell types | Males and females with tumors |
| | expressing p53 | expressing p53 |
| HPV 16 E7 | Cancers of the cervix, | HPV infected males and females |
| protein | vulva, vagina, penis, | |
| | oropharynx and anus. | |
| HPV L2 | Cancers of the cervix, | HPV infected males and females |
| | vulva, vagina, penis, | |
| | oropharynx and anus. | |
| PD-L1 | Cancer types PD-L1 | Males and females with tumors |
| | | expressing PD-L1 |
| PD-L1 | Cancer types PD1 | Males and females with tumors |
| | | expressing PD1 |
| CTLA-4 | Cancer types CTLA-4 | Males and females with tumors |
| | | expressing CTLA-4 |
| hCG | Cancer cell types | Males and females with tumors |
| | expressing hCG | expressing hCG |
| Fel d1 | Cat allergy | Males and females allergic to cats |
| (IHNV) G-protein | Infectious | Salmon and trout infected with IHNV |
| | haematopoietic necrosis | |
| | (IHN) | |
| SARS-CoV-2 | SARS-CoV-2 infection | Males and females |
| spike protein | | |
| RBD | | |

TABLE 3
Non-exhaustive list of diseases/medical indications and target
antigen/organisms of the present VLP/nanoparticle vaccine.

| Disease: | Target antigen/Organism: |
|---|---|
| Cancer: | Her2/Neu (ERBB2)/Homo Sapiens |
| | Survivin (Baculoviral IAP repeat-containing |
| | protein 5)/Homo Sapiens |
| | GD2/Homo Sapiens |
| | EGF-R/Homo Sapiens |
| | CEA/Homo Sapiens |
| | CD52/Homo Sapiens |
| | human melanoma protein gp100/Homo Sapiens |
| | human melanoma protein melan-A/MART-1/Homo Sapiens |
| | tyrosinase/Homo Sapiens |
| | NA17-A nt protein/Homo Sapiens |
| | MAGE-3 protein/Homo Sapiens |
| | p53 protein/Homo Sapiens |
| | HPV 16 E7 protein/Human papillomavirus |
| | HPV L2 protein/Human papillomavirus |
| | PD1/Homo Sapiens |
| | PD-L1/Homo Sapiens |
| | CTLA-4/Homo Sapiens |
| | hCG/Homo Sapiens |
| | (IHNV) G-protein/Infectious haematopoietic necrosis virus |
| Cardiovascular disease: | PCSK9 (Proprotein convertase subtilisin/kexin type 9)/Homo |
| | Sapiens |
| Asthma/Allergies: | IL-5 (Interleukin-5)/Homo Sapiens |
| | Fel d1/Felis catus |
| | IL-17/Homo sapiens |
| | IL-13/Homo sapiens |
| | IL-1B/Homo sapiens |
| | IgE/Homo sapiens |
| Tuberculosis: | Ag85A (Diacylglycerol cyltransferase/mycolyltransferase)/ |
| | Mycobacterium tuberculosis |
| Malaria: | Reticulocyte-binding protein homologue 5 (PfRH5)/Plasmodium |
| | falciparum |
| | VAR2CSA (domain, ID1-ID2a)/Plasmodium falciparum |
| | CIDR1a domain of PfEMP1, Plasmodium falciparum |
| | Glutamate rich protein (GLURP)/Plasmodium falciparum |
| | Merozoite surface protein 3 (MSP3)/Plasmodium falciparum |
| | 25 kDa ookinete surface antigen (Pfs25)/Plasmodium |
| | falciparum |
| | Circumsporozoite protein (CSP)/Plasmodium falciparum |
| | Schizont egress antigen-1 (PfSEA-1)/Plasmodium falciparum |
| Multiple sclerosis | CD52/Homo sapiens |
| Contraception | hCG |
| Influenza | HA |

### Cancer and Associated Antigens

In 2012 more than 14 million adults were diagnosed with cancer and there were more than 8 million deaths from cancer, globally. Consequently, there is a need for efficient cancer therapeutics.

One characteristic of cancer cells is abnormal expression levels of genes and proteins. One example of a cancer associated gene is HER2, which is overexpressed in 20% of all breast cancers and is associated with increased metastatic potential and poor patient survival. Although cancer cells express cancer associated antigens in a way that immunologically distinguishes them from normal cells, most cancer associated antigens are only weakly immunogenic because most cancer associated antigens are “self” proteins which are generally tolerated by the host. The present compositions can be used to express, in a cell of a subject, particles displaying an antigen capable of activating the immune system to react against for example cancer associated antigens and overcome the immunological tolerance to such antigens. Different cancers are characterized by having different cancer associated antigens. Survivin is regarded to be overexpressed in most cancer cells and could also be used in the present invention. Therefore the present invention may be used in treatment/prophylaxis of most types of cancers that overexpress a tumor associated antigen.

Thereby the present invention provides compositions capable of activating the immune system to react against for example cancer associated antigens and overcome immunological tolerance to such antigens. In an embodiment the present compositions can be used for prophylaxis and/or treatment of the cancer which the antigen is associated with.

In another embodiment the present invention is used in treatment/prophylaxis of any type of cancer which overexpresses an antigen. The type of cancer which the invention may be used against is determined by the choice of antigen.

It is known that oncoviruses can cause cancer. Therefore in an embodiment the vaccine of the present invention comprises an oncovirus associated antigen linked to a particle-forming protein.

In a further embodiment the present compositions can be used for prophylaxis and/or treatment of the cancer which the antigen is associated with.

In an embodiment the antigen is a protein or peptide or an antigenic fragment of a polypeptide associated with a cancer selected from the group comprising of adrenal cancer, anal cancer, bile duct cancer, bladder cancer, bone cancer, brain/CNS tumors in adults, brain/CNS tumors in children, breast cancer, breast cancer in men, cancer in adolescents, cancer in children, cancer in young adults, cancer of unknown primary (CUP), Castleman disease, cervical cancer, colon/rectum cancer, endometrial cancer, esophagus cancer, Ewing family of tumors, eye cancer, gallbladder cancer, gastrointestinal carcinoid tumors, gastrointestinal stromal tumor, gestational trophoblastic disease, Hodgkin disease, Kaposi sarcoma, kidney cancer, laryngeal and hypopharyngeal cancer, leukemia, acute lymphocytic in adults, leukemia, acute myeloid leukemia, chronic lymphocytic leukemia, chronic myeloid leukemia, chronic myelomonocytic leukemia, leukemia in children, liver cancer, lung cancer, non-small cell lung cancer, small cell lung cancer, lung carcinoid tumor, lymphoma, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, non-Hodgkin lymphoma in children, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, adult soft tissue cancer sarcoma, skin cancer, basal and squamous cell skin cancer, melanoma skin cancer, Merkel cell skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor.

In some embodiments the cancer is selected from the group consisting of breast cancer, gastric cancer, ovarian cancer, and uterine serous carcinoma.

Linking the Her2/Neu (ERBB2) and/or Survivin or an antigenic fragment hereof to the VLP or nanoparticle as described herein forms a VLP or nanoparticle which is capable of activating the immune system to react against for example cells with high Her2/Neu (ERBB2) and/or Survivin expression and overcome immunological tolerance. In an embodiment the Her2/Neu (ERBB2) and/or Survivin can be used for prophylaxis and/or treatment of the herein disclosed cancer disease and/or other cancer diseases. Using a similar reasoning other cancer disease associated antigens may be used against any cancer disease. Such antigens may be chosen from the group consisting of interleukin-17, hemagglutinin, GD2, EGF-R, CEA, CD52, CD21, human melanoma protein gp100, human melanoma protein melan-A/MART1, tyrosinase, NA17-A nt, MAGE-3, HPV 16 E7, HPV L2, PD1, PD-L1, CTLA-4, p53, hCG, Fel d1 and (IHNV) G-protein.

In an embodiment the antigen of the present invention is Her2/Neu (ERBB2) and/or Survivin or an antigenic fragment hereof, wherein the antigen is associated with and directed against at least one of the herein disclosed types of cancers. In an embodiment the antigen of the present disclosure is interleukin-17, hemagglutinin, GD2, EGF-R, CEA, CD52, CD21, human melanoma protein gp100, human melanoma protein melan-A/MART1, tyrosinase, NA17-A nt, MAGE-3, HPV 16 E7, HPV L2, PD1, PD-L1, CTLA-4, HPV L2, PD1, PD-L1, CTLA-4, p53, hCG, Fel d1 and (IHNV) G-protein or an antigenic fragment thereof, wherein the antigen is associated with and directed against at least one of the herein disclosed types of cancers.

### Cardiovascular Diseases and Associated Antigens

An estimated 17.3 million people died from cardiovascular diseases in 2008, representing 30% of all global deaths. Addressing risk factors such as tobacco use, unhealthy diet and obesity, physical inactivity, high blood pressure, diabetes and raised lipids are important for prevention of cardiovascular diseases. However, the need for preventive pharmaceutical measures is increasingly important. The present invention may be used in treatment/prophylaxis of most types of cardiovascular diseases. The type of cardiovascular disease which the invention may be used against is decided by the choice of antigen.

In an embodiment of the invention the antigen is a protein or peptide or an antigenic fragment of a polypeptide associated with a disease selected from the group comprising a lipid disorder such as hyperlipidemia, type I, type II, type Ill, type IV, or type V hyperlipidemia, secondary hypertriglyceridemia, hypercholesterolemia, familial hypercholesterolemia, xanthomatosis, cholesterol acetyltransferase deficiency, an ateriosclerotic condition (e.g., atherosclerosis), a coronary artery disease, a cardiovascular disease.

In an embodiment of the invention the antigen is a protein or peptide or an antigenic fragment of a polypeptide associated with a cardiovascular disease. In a further embodiment the cardiovascular disease is selected from the group consisting of dyslipidemia, atherosclerosis, and hypercholesterolemia.

One example of a polypeptide associated with a cardiovascular disease is PCSK9 which acts in cholesterol homeostasis. Blockage of PCSK9 has medical significance and can lower the plasma and/or serum low-density lipoprotein cholesterol (LDL-C) levels. Reducing LDL-C reduces the risk of for example heart attacks.

Linking the PCSK9 antigen to the VLP or nanoparticle forms a PCSK9-VLP/nanoparticle based vaccine which is capable of activating the immune system to produce antibodies that bind PCSK9 and either clear PCSK9 from the bloodstream or hinders binding of PCSK9 to the LDL receptor, thereby lowering the LDL-C levels and the risk of heart attacks. In an embodiment, the present compositions can be used for prophylaxis and/or treatment of the herein disclosed cardiovascular disease and/or other cardiovascular diseases. Using a similar reasoning other cardiovascular disease associated antigens may be used against any cardiovascular disease.

In a preferred embodiment the antigen comprises PCSK9 or an antigenic fragment hereof, wherein the antigen is associated with and directed against at least one of the herein disclosed cardiovascular disease and/or other cardiovascular diseases.

Another example of a polypeptide associated with a cardiovascular disease is ANGPTL3 which acts in cholesterol homeostasis. Blockage of ANGPTL3 has medical significance and can lower the plasma and/or serum low-density lipoprotein cholesterol (LDL-C) levels. Reducing LDL-C reduces the risk of for example heart attacks.

Linking the ANGPTL3 antigen to the VLP or nanoparticle forms a ANGPTL3-VLP/nanoparticle based vaccine which is capable of activating the immune system to produce antibodies that bind ANGPTL3 and either clear ANGPTL3 from the bloodstream or hinders binding of ANGPTL3 to the LDL receptor, thereby lowering the LDL-C levels and the risk of heart attacks. In an embodiment, the present compositions can be used for prophylaxis and/or treatment of the herein disclosed cardiovascular disease and/or other cardiovascular diseases. Using a similar reasoning other cardiovascular disease associated antigens may be used against any cardiovascular disease.

In a preferred embodiment the antigen comprises ANGPTL3 or an antigenic fragment hereof, wherein the antigen is associated with and directed against at least one of the herein disclosed cardiovascular disease and/or other cardiovascular diseases.

### Immune-Inflammatory Diseases and Associated Antigens

The prevalence of immune-inflammatory diseases worldwide is rising dramatically in both developed and developing countries. According to World Health Organization statistics, hundreds of millions of subjects in the world suffer from allergic rhinitis and it is estimated that 300 million have asthma markedly affecting the quality of life of these individuals and negatively impacting the socio-economic welfare of society.

Interleukin 5 (IL-5) has been shown to play an instrumental role in eosinophilic inflammation in various types of allergies, including severe eosinophilic asthma. Eosinophils are regulated in terms of their recruitment, activation, growth, differentiation and survival by IL-5 which, consequently, has identified this cytokine as a primary target for therapeutic interventions.

Linking an IL-5 antigen or a fragment hereof to the particle-forming protein of the present invention forms an IL-5-VLP/nanoparticle based vaccine which is capable of activating the immune system to react against IL-5. Consequently an IL-5-based composition described in the present invention may be used in the treatment/prophylaxis of eosinophilic asthma or other immune-inflammatory diseases. Other immune-inflammatory disease associated antigens (e.g. IgE or interleukin 17 or IL-17) may be used by the present invention using a similar reasoning. Consequently an IL-17-based vaccine described in the present invention may be used in the treatment/prophylaxis of eosinophilic asthma or other immune-inflammatory diseases. The type of asthma or allergy or other immune-inflammatory disease which the invention may be used against is decided by the choice of antigen. In an embodiment the antigen is a protein or peptide or an antigenic fragment of a polypeptide associated with one or more asthma or immune-inflammatory diseases disclosed herein. In a preferred embodiment the asthma or immune-inflammatory disease is selected from the group consisting of eosinophilic asthma, allergy, nasal polyposis, atopic dermatitis, eosinophilic esophagitis, hypereosinophilic syndrome, and Churg-Strauss syndrome.

In a preferred embodiment the antigen comprises IL-5, IL-17 or an antigenic fragment hereof, wherein the antigen is associated with and directed against at least one of the herein disclosed asthma or allergy diseases and/or other immune-inflammatory diseases.

The IRMM as disclosed herein may be particularly useful in treatment of autoimmune diseases or immune-mediated disorders, such as by promoting a tolerogenic response towards the antigen linked to the particle. Such a response may be mediated through induction of an antigen-specific T helper type 1 (Th2) response and/or suppression of an antigen-specific T helper type 1 (Th1) response.

### Infectious Diseases and Associated Antigens

Tuberculosis and malaria are two major infectious diseases. In 2012, an estimated 207 million cases of malaria occurred which resulted in more than 500.000 deaths. Also in 2012, an estimated 8.6 million people developed tuberculosis and 1.3 million died from the disease. The current methods of treatment are insufficient and some have resulted in drug resistance. Consequently there is a need for new and efficient drugs for treatment/prophylaxis of tuberculosis and malaria. Linking a malaria or tuberculosis associated-antigen or a fragment hereof to the VLP or nanoparticle of the present invention forms a VLP or nanoparticle based vaccine which is capable of activating the immune system to react against for example malaria or tuberculosis. Using a similar line of reasoning the present invention may be used in treatment/prophylaxis of most infectious disease. The type of infectious disease which the invention may be used against is decided by the choice of antigen.

The year 2020 has been marked by the SARS-CoV-2/COVID-19 pandemic, resulting in millions of infected subjects worldwide, and this infectious disease is thus of great interest. Influenza is another infectious disease of interest.

In an embodiment the antigen fused to the second peptide tag of the present invention is a protein or peptide or an antigenic fragment of a polypeptide associated with an infectious disease such as tuberculosis, schistosomiasis and/or malaria.

In one embodiment an antigen from Plasmodium falciparum is fused to the second peptide or affinity tag for use in treatment/prophylaxis of malaria.

In a further embodiment an antigen from Mycobacterium tuberculosis is fused to the second peptide or affinity tag for use in treatment/prophylaxis of tuberculosis.

In a further embodiment the antigen is selected from the group consisting of Ag85A from Mycobacterium tuberculosis, PfRH5 from Plasmodium falciparum, VAR2CSA (domain, ID1-ID2a) from Plasmodium falciparum, CIDR1a domain, of PfEMP1 from Plasmodium falciparum, GLURP from Plasmodium falciparum, MSP3 from Plasmodium falciparum, Pfs25 from Plasmodium falciparum, CSP from Plasmodium falciparum, and PfSEA-1 from Plasmodium falciparum or an antigenic fragment of the disclosed antigens. In another embodiment the antigen comprises a fusion construct between MSP3 and GLURP (GMZ2) from Plasmodium falciparum.

In a further embodiment the antigen is a hemagglutinin (HA) antigen from the influenza virus or an antigenic fragment thereof.

In some embodiments, the antigen is an antigen from a flatworm from the genus Schistosoma for use in treatment/prophylaxis of schistosomiasis.

In another embodiment the antigen of the present invention comprises a protein, or an antigenic fragment hereof, from the pathogenic organism which causes the infectious disease.

In one embodiment, the antigen is a protein, peptide and/or an antigenic fragment of Severe Acute Respiratory Syndrome Coronavirus 2 (SARS-CoV-2). Thus, in some embodiments, the antigen is a protein, peptide and/or an antigenic fragment of a SARS-CoV-2 envelope protein. In some embodiments, the antigen is a protein, peptide and/or an antigenic fragment of a SARS-CoV-2 spike protein, such as the SARS-CoV-2 spike protein RBD (SEQ ID NO: 145, DNA sequence SEQ ID NO: 146). In some embodiments, the antigen is a protein, peptide and/or an antigenic fragment of a SARS-CoV-2 nucleocapsid protein. In some embodiments, the antigen is a protein, peptide and/or an antigenic fragment of a SARS-CoV-2 envelope protein. In specific embodiments, the antigen is amino acids 319-591 of the SARS-CoV-2 spike protein RBD (GenBank accession number: QIA20044.1). Said antigen may be fused to a catcher or a tag, and may further comprise a C-tag purification tag such as SEQ ID NO: 187 (EPEA), optionally via a linker, such as SEQ ID NO: 188 (GSGS) or SEQ ID NO: 189 (GSGTAGGGSGS).

In one embodiment the antigen is a protein, peptide and/or an antigenic fragment of an influenza virus.

### Effects of the Compositions and Examples of Specific Compositions

The invention provides compositions comprising a particle-forming protein, an IRMM and an antigen, wherein the IRMM is able to modulate the antigen-specific immune response. As disclosed herein above, the IRMM and the antigen may be linked to the particle-forming protein in various ways, such as by direct fusion, affinity conjugation, and/or through a first peptide tag forming an isopeptide bond with a second peptide and optionally a third peptide tag forming an isopeptide bond with a fourth peptide tag.

In a specific embodiment, the particle-forming protein is AP205 (SEQ ID NO: 143), the first peptide tag is a RumTrunk tag (SEQ ID NO: 14), the second peptide tag is a MoonCake (SEQ ID NO: 115) and the IRMM is the CD180 agonist peptide according to SEQ ID NO: 123, wherein
i) the second peptide tag is fused to the antigen, optionally by a linker; and
ii) the first peptide tag and the IRMM are fused to the particle-forming protein, optionally by separate linkers.

In another specific embodiment, the particle-forming protein is AP205 (SEQ ID NO: 143), the first peptide tag is a RumTrunk tag (SEQ ID NO: 14), the second peptide tag is a MoonCake (SEQ ID NO: 115) and the IRMM is the C3 binding moiety according to SEQ ID NO: 125, wherein
i) the second peptide tag is fused to the antigen, optionally by a linker; and
ii) the first peptide tag and the IRMM are fused to the particle-forming protein, optionally by separate linkers.

In a further specific embodiment, the particle-forming protein is AP205 (SEQ ID NO: 143), the first peptide tag is a MoonCake (SEQ ID NO: 115) and the IRMM is the CD180 agonist peptide according to SEQ ID NO: 123, or the dendritic cell receptor binding peptide according to SEQ ID NO: 127, wherein
i) the first peptide tag is fused to the particle-forming protein, optionally by a linker; and
ii) the IRMM is fused to the first peptide tag, optionally by a linker.

In another specific embodiment, the particle-forming protein is AP205 (SEQ ID NO: 143), the first peptide tag is a MoonCake (SEQ ID NO: 115) and the IRMM is selected from the group consisting of a CD180 agonist peptide, such as the peptide according to SEQ ID NO: 123, a dendritic cell receptor binding peptide, such as the peptide according to SEQ ID NO: 127, a mast cell-activating peptide, such as mastoparan-7 (M7) according to SEQ ID NO: 129 or SEQ ID NO: 131, and a TLR2 agonist peptide, such as a peptide according to SEQ ID NO: 133, wherein
i) the first peptide tag is fused to the particle-forming protein, optionally by a linker; and
ii) the IRMM is fused to the particle-forming protein, optionally by a linker.

In a further specific embodiment, the particle-forming protein is AP205 (SEQ ID NO: 143), the first peptide tag is a RumTrunk D9N tag (SEQ ID NO: 13) and the IRMM is selected from the group consisting of a CD180 agonist peptide, such as the peptide according to SEQ ID NO: 123, a dendritic cell receptor binding peptide, such as the peptide according to SEQ ID NO: 127, a mast cell-activating peptide, such as mastoparan-7 (M7) according to SEQ ID NO: 129 or SEQ ID NO: 131, and a TLR2 agonist peptide, such as a peptide according to SEQ ID NO: 133, wherein
i) the first peptide tag is fused to the particle-forming protein, optionally by a linker; and
ii) the IRMM is fused to the particle-forming protein, optionally by a linker.

In a specific embodiment, the particle-forming protein is AP205 (SEQ ID NO: 143), the first peptide tag is a RumTrunk tag (SEQ ID NO: 14), the second peptide tag is a MoonCake (SEQ ID NO: 115), the antigen is the SARS-CoV-2 spike protein RBD (SEQ ID NO: 145) and the IRMM is the CD180 agonist peptide according to SEQ ID NO: 123, wherein
i) the second peptide tag is fused to the antigen, optionally by a linker; and
ii) the first peptide tag and the IRMM are fused to the particle-forming protein, optionally by separate linkers.

In another specific embodiment, the particle-forming protein is AP205 (SEQ ID NO: 143), the first peptide tag is a RumTrunk tag (SEQ ID NO: 14), the second peptide tag is a MoonCake (SEQ ID NO: 115), the antigen is the SARS-CoV-2 spike protein RBD (SEQ ID NO: 145) and the IRMM is the C3 binding moiety according to SEQ ID NO: 125, wherein
i) the second peptide tag is fused to the antigen, optionally by a linker; and
ii) the first peptide tag and the IRMM are fused to the particle-forming protein, optionally by separate linkers.

In a further specific embodiment, the particle-forming protein is AP205 (SEQ ID NO: 143), the first peptide tag is a MoonCake (SEQ ID NO: 115), the antigen is the SARS-CoV-2 spike protein RBD (SEQ ID NO: 145) and the IRMM is the CD180 agonist peptide according to SEQ ID NO: 123 or the dendritic cell receptor binding peptide according to SEQ ID NO: 127, wherein
i) the first peptide tag is fused to the particle-forming protein, optionally by a linker; and
ii) the IRMM is fused to the first peptide tag, optionally by a linker.

In another specific embodiment, the particle-forming protein is AP205 (SEQ ID NO: 143), the first peptide tag is a MoonCake (SEQ ID NO: 115), the antigen is the SARS-CoV-2 spike protein RBD (SEQ ID NO: 145) and the IRMM is selected from the group consisting of a CD180 agonist peptide, such as the peptide according to SEQ ID NO: 123, a dendritic cell receptor binding peptide, such as the peptide according to SEQ ID NO: 127, a mast cell-activating peptide, such as mastoparan-7 (M7) according to SEQ ID NO: 129 or SEQ ID NO: 131, and a TLR2 agonist peptide, such as a peptide according to SEQ ID NO: 133, wherein
i) the first peptide tag is fused to the particle-forming protein, optionally by a linker; and
ii) the IRMM is fused to the particle-forming protein, optionally by a linker.

In a further specific embodiment, the particle-forming protein is AP205 (SEQ ID NO: 143), the first peptide tag is a RumTrunk D9N tag (SEQ ID NO: 13), the antigen is the SARS-CoV-2 spike protein RBD (SEQ ID NO: 145) and the IRMM is selected from the group consisting of a CD180 agonist peptide, such as the peptide according to SEQ ID NO: 123, a dendritic cell receptor binding peptide, such as the peptide according to SEQ ID NO: 127, a mast cell-activating peptide, such as mastoparan-7 (M7) according to SEQ ID NO: 129 or SEQ ID NO: 131, and a TLR2 agonist peptide, such as a peptide according to SEQ ID NO: 133, wherein
i) the first peptide tag is fused to the particle-forming protein, optionally by a linker; and
ii) the IRMM is fused to the particle-forming protein, optionally by a linker.

In a specific embodiment, the particle-forming protein is AP205 (SEQ ID NO: 143), the first peptide tag is a RumTrunk tag (SEQ ID NO: 14), the second peptide tag is a MoonCake (SEQ ID NO: 115), the antigen is HER2 and the IRMM is the CD180 agonist peptide according to SEQ ID NO: 123, wherein
i) the second peptide tag is fused to the antigen, optionally by a linker; and
ii) the first peptide tag and the IRMM are fused to the particle-forming protein, optionally by separate linkers.

In another specific embodiment, the particle-forming protein is AP205 (SEQ ID NO: 143), the first peptide tag is a RumTrunk tag (SEQ ID NO: 14), the second peptide tag is a MoonCake (SEQ ID NO: 115), the antigen is HER2 and the IRMM is the C3 binding moiety according to SEQ ID NO: 125, wherein
i) the second peptide tag is fused to the antigen, optionally by a linker; and
ii) the first peptide tag and the IRMM are fused to the particle-forming protein, optionally by separate linkers.

In a further specific embodiment, the particle-forming protein is AP205 (SEQ ID NO: 143), the first peptide tag is a MoonCake (SEQ ID NO: 115), the antigen is HER2 and the IRMM is the CD180 agonist peptide according to SEQ ID NO: 123 or the dendritic cell receptor binding peptide according to SEQ ID NO: 127, wherein
i) the first peptide tag is fused to the particle-forming protein, optionally by a linker; and
ii) the IRMM is fused to the first peptide tag, optionally by a linker.

In another specific embodiment, the particle-forming protein is AP205 (SEQ ID NO: 143), the first peptide tag is a MoonCake (SEQ ID NO: 115), the antigen is HER2 and the IRMM is selected from the group consisting of a CD180 agonist peptide, such as the peptide according to SEQ ID NO: 123, a dendritic cell receptor binding peptide, such as the peptide according to SEQ ID NO: 127, a mast cell-activating peptide, such as mastoparan-7 (M7) according to SEQ ID NO: 129 or SEQ ID NO: 131, and a TLR2 agonist peptide, such as a peptide according to SEQ ID NO: 133, wherein
i) the first peptide tag is fused to the particle-forming protein, optionally by a linker; and
ii) the IRMM is fused to the particle-forming protein, optionally by a linker.

In a further specific embodiment, the particle-forming protein is AP205 (SEQ ID NO: 143), the first peptide tag is a RumTrunk D9N tag (SEQ ID NO: 13), the antigen is HER2 and the IRMM is selected from the group consisting of a CD180 agonist peptide, such as the peptide according to SEQ ID NO: 123, a dendritic cell receptor binding peptide, such as the peptide according to SEQ ID NO: 127, a mast cell-activating peptide, such as mastoparan-7 (M7) according to SEQ ID NO: 129 or SEQ ID NO: 131, and a TLR2 agonist peptide, such as a peptide according to SEQ ID NO: 133, wherein
i) the first peptide tag is fused to the particle-forming protein, optionally by a linker; and
ii) the IRMM is fused to the particle-forming protein, optionally by a linker.

In a specific embodiment, the particle-forming protein is AP205 (SEQ ID NO: 143), the first peptide tag is a RumTrunk tag (SEQ ID NO: 14), the second peptide tag is a MoonCake (SEQ ID NO: 115), the antigen is VAR2CSA and the IRMM is the CD180 agonist peptide according to SEQ ID NO: 123, wherein
i) the second peptide tag is fused to the antigen, optionally by a linker; and
ii) the first peptide tag and the IRMM are fused to the particle-forming protein, optionally by separate linkers.

In another specific embodiment, the particle-forming protein is AP205 (SEQ ID NO: 143), the first peptide tag is a RumTrunk tag (SEQ ID NO: 14), the second peptide tag is a MoonCake (SEQ ID NO: 115), the antigen is VAR2CSA and the IRMM is the C3 binding moiety according to SEQ ID NO: 125, wherein
i) the second peptide tag is fused to the antigen, optionally by a linker; and
ii) the first peptide tag and the IRMM are fused to the particle-forming protein, optionally by separate linkers.

In a further specific embodiment, the particle-forming protein is AP205 (SEQ ID NO: 143), the first peptide tag is a MoonCake (SEQ ID NO: 115), the antigen is VAR2CSA and the IRMM is the CD180 agonist peptide according to SEQ ID NO: 123 or the dendritic cell receptor binding peptide according to SEQ ID NO: 127, wherein
i) the first peptide tag is fused to the particle-forming protein, optionally by a linker; and
ii) the IRMM is fused to the first peptide tag, optionally by a linker.

In another specific embodiment, the particle-forming protein is AP205 (SEQ ID NO: 143), the first peptide tag is a MoonCake (SEQ ID NO: 115), the antigen is VAR2CSA and the IRMM is selected from the group consisting of a CD180 agonist peptide, such as the peptide according to SEQ ID NO: 123, a dendritic cell receptor binding peptide, such as the peptide according to SEQ ID NO: 127, a mast cell-activating peptide, such as mastoparan-7 (M7) according to SEQ ID NO: 129 or SEQ ID NO: 131, and a TLR2 agonist peptide, such as a peptide according to SEQ ID NO: 133, wherein
i) the first peptide tag is fused to the particle-forming protein, optionally by a linker; and
ii) the IRMM is fused to the particle-forming protein, optionally by a linker.

In a further specific embodiment, the particle-forming protein is AP205 (SEQ ID NO: 143), the first peptide tag is a RumTrunk D9N tag (SEQ ID NO: 13), the antigen is VAR2CSA and the IRMM is selected from the group consisting of a CD180 agonist peptide, such as the peptide according to SEQ ID NO: 123, a dendritic cell receptor binding peptide, such as the peptide according to SEQ ID NO: 127, a mast cell-activating peptide, such as mastoparan-7 (M7) according to SEQ ID NO: 129 or SEQ ID NO: 131, and a TLR2 agonist peptide, such as a peptide according to SEQ ID NO: 133, wherein
i) the first peptide tag is fused to the particle-forming protein, optionally by a linker; and
ii) the IRMM is fused to the particle-forming protein, optionally by a linker.

Two different IRMMs comprised in the same composition when administered to a subject may lead to a better modulation of the immune response than the sum of the effects of each IRMM when administered alone, i.e. a synergistic effect. Thus, in some embodiments, the composition comprises at least two IRMMs, which synergistically modulate the immune response, such as by activation of multiple TLRs or by activation of surface receptors on multiple different types of immune cells.

In some embodiments, the composition comprises at least a first and a second IRMMs, wherein the first IRMM is an agonist of TLR4, such as a peptide according to SEQ ID NO: 121, and the second IRMM is an agonist of CD180, such as a peptide according to SEQ ID NO: 123.

In some embodiments, the composition comprises at least a first and a second IRMMs, wherein the first IRMM is an agonist of TLR4, such as a peptide according to SEQ ID NO: 121, and the second IRMM is an agonist of TLR2, such as a peptide according to SEQ ID NO: 133.

Administration of the composition as disclosed herein comprising said IRMM leads to an altered antigen-specific immune response compared to administration of an identical composition not comprising said IRMM.

In some embodiments, administration of the composition increases or decreases an antibody immune response, such as by increasing, inhibiting or decreasing antigen-specific antibody production, compared to administering the same composition not comprising said IRMM.

In some embodiments, administration of the composition increases total antigen-specific IgG production, compared to administering the same composition not comprising said IRMM.

In some embodiments, administration of the composition increases or decreases a cellular immune response, such as by increasing, inhibiting or decreasing an antigen-specific cellular immune response, compared to administering the same composition not comprising said IRMM.

In some embodiments, administration of the composition directs an antigen-specific immune response toward a specific IgG profile, such as an increased human IgG1 response or an increased human IgA response, compared to administering the same composition not comprising said IRMM.

In some embodiments, administration of the composition alters the ratio of different types of antigen-specific immunoglobulins, such as IgG vs IgA vs IgM, compared to administering the same composition not comprising said IRMM.

In some embodiments, administration of the composition leads to an altered ratio of induction of antigen-specific CD4+ T cells vs antigen-specific CD8+ T cells, compared to administering the same composition not comprising said IRMM. In some embodiments, administration of the composition leads to an increased of induction of antigen-specific CD4+ T cells vs antigen-specific CD8+ T cells, compared to administering the same composition not comprising said IRMM. In some embodiments, administration of the composition leads to a decreased ratio of induction of antigen-specific CD4+ T cells vs antigen-specific CD8+ T cells, compared to administering the same composition not comprising said IRMM.

### Systems

Herein is also provided a system comprising:
i) a particle-forming protein fused to a first peptide tag and, optionally, to a third peptide tag, optionally by a linker;
ii) said antigen fused to a second peptide tag, optionally by a linker; and
iii) at least one immune response modulating moiety (IRMM),
   wherein
a) i), ii) and iii) are in the form of one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond or by affinity conjugation, optionally by a linker; or
   ii. is fused to a fourth peptide tag, optionally by a linker; and
c) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
      whereby i)-ii)-iii) form a particle displaying said antigen and said at least one IRMM.

Also provided herein is a system comprising:
i) a particle-forming protein fused to said antigen, optionally by a linker, and, optionally, to a third peptide tag, optionally by a linker; and
ii) at least one immune response modulating moiety (IRMM)
   wherein
a) i) and ii) are in the form of one or more polynucleotides encoding i) and ii);
b) said at least one IRMM
   i) is linked to said particle-forming protein or said antigen by fusion, by an isopeptide bond, by an ester bond or by affinity conjugation, optionally by a linker; or
   ii) is fused to a fourth peptide tag, optionally by a linker; and
c) the at least one IRMM and the particle-forming protein are capable of being linked via an isopeptide bond, via an ester bond, via chemical crosslinking or via affinity conjugation between the third and the fourth peptide tags when present,
   whereby i)-ii) form a particle displaying said antigen and said at least one IRMM.

In some embodiments, the particle-forming protein fused to said first peptide tag and, optionally, to said third peptide tag, is encoded by a first polynucleotide.

In some embodiments, the antigen fused to said second peptide tag is encoded by a second polynucleotide.

In some embodiments, the at least one IRMM fused to a fourth peptide tag is encoded by a third polynucleotide. In some embodiments, the first and/or second polynucleotide further encodes said at least one IRMM.

In some embodiments, the first polynucleotide comprises or consists of a first polynucleotide encoding said particle-forming protein comprising said first peptide tag and said at least one IRMM, and the second polynucleotide comprises or consists of a second polynucleotide encoding said antigen comprising said second peptide.

In some embodiments, the first polynucleotide comprises or consists of a first polynucleotide encoding said particle-forming protein comprising said first peptide tag and said third protein tag, the second polynucleotide comprises or consists of a second polynucleotide encoding said antigen comprising said second peptide tag, and the third polynucleotide comprises or consists of a third polynucleotide encoding said at least one IRMM fused to said fourth peptide tag.

In some embodiments, the first polynucleotide comprises or consists of a first polynucleotide encoding said particle-forming protein comprising said first peptide tag, and the second polynucleotide comprises or consists of a second polynucleotide encoding said antigen comprising said second peptide tag and said at least one IRMM.

The system may consist of or comprise a polycistronic RNA construct and/or a DNA construct, from which the transcribed mRNA is polycistronic. Thus, in some embodiments, the first, second and, optionally, third polynucleotides of the system are encoded on the same ribonucleic acid molecule. In some embodiments, the first, the second and, optionally, the third polynucleotides of the system lie within the same open reading frame, whereby only one promoter sequence is needed to transcribe the polynucleotides. In some embodiments, the first, the second and, optionally, the third polynucleotides of the system lie within separate open reading frames and may thus be regulated by separate promoters.

The first peptide tag, the second peptide tag, the third peptide tag, the fourth peptide tag, the particle-forming protein, the antigen and/or the IRMM may be as defined herein elsewhere.

The term “system” refers to a genetic construct designed to produce a protein and/or an RNA inside a cell. Thus, the system may comprise RNA and/or DNA, which is translated or transcribed to a protein or DNA, respectively, inside the cell.

The system may comprise the sequences necessary for gene expression in the cell. These may include a promoter, a translation initiation sequence such as a ribosomal binding site, a start codon, a termination codon, and a transcription termination sequence. There are differences in the enzymes responsible for protein synthesis between prokaryotes and eukaryotes, therefore the expression vectors must comprise elements for expression that are appropriate for the chosen host. For example, prokaryotic systems may comprise a Shine-Dalgarno sequence at the translation initiation site for the binding of ribosomes, while eukaryotic systems may contain a Kozak consensus sequence.

The system may additionally comprise a marker, such as a selectable marker, i.e. a gene that confers a trait suitable for artificial selection, whereby cells comprising the system may be selected for, or a screenable marker, such as a reporter gene, i.e. a gene that allows for differentiation between cells comprising or not comprising the system, whereby cells comprising the system may be identified. Examples of such markers include antibiotic resistance genes, auxotrophic markers and genes expressing detectable compounds, such as coloured and/or fluorescent compounds.

In some embodiments, the first polynucleotide, the second polynucleotide and the third polynucleotide are all DNA polynucleotides. In some embodiments, the first polynucleotide, the second polynucleotide and the third polynucleotide are all RNA polynucleotides.

In some embodiments, at least one of the first polynucleotide the second polynucleotide and the third polynucleotide is a DNA polynucleotide and the other(s) is/are RNA polynucleotide(s).

In some embodiments, the first, second and third polynucleotides are comprised within one vector such as a viral vector or a plasmid. In some embodiments, the first and second polynucleotides are comprised within two vectors such as two viral vectors; two plasmids; or one viral vector and one plasmid. In some embodiments, the first, second and third polynucleotides are comprised within three vectors such as three viral vectors; three plasmids; or at least one viral vector and at least one plasmid.

In some embodiments, the viral vector is an adenoviral vector or a modified vaccinia Ankara (MVA) vector. In some embodiments, the system comprises or consists of an mRNA.

In some embodiments, the system comprises or consists of a plasmid. In some embodiments, said plasmid is pVAX1.

The first polynucleotide, the second polynucleotide and/or the third polynucleotide may be under the control of a promoter, such as an inducible promoter, e.g. a vitamin D-inducible promoter, or a constitutive promoter. The first, second and/or the third polynucleotide may each be under the control of a first, second and/or third promoter, respectively, which may be identical or different. They may also be under the control of a single promoter.

The first, the second and, optionally, the third polynucleotides of the system may be comprised within the same molecule. The first, second and, optionally, third polynucleotides of the system may alternatively be comprised within different molecules, such as within two, three or more separate molecules.

The first, the second and/or the third polynucleotide may further comprise a secretion or excretion signal to obtain a fusion protein comprising such a signal, whereby the particle-forming protein fused to the first peptide tag and, optionally, the third peptide tag, the IRMM fused to the fourth peptide tag, and/or the antigen fused to the second peptide tag is secreted or excreted from the endoplasmic reticulum and optionally also from the cell. In some embodiments, the secretion or excretion signal comprises or consists of SEQ ID NO: 79, SEQ ID NO: 80, SEQ ID NO: 81, SEQ ID NO: 82, SEQ ID NO: 83 or SEQ ID NO: 84.

The present expressions systems can be used for prophylaxis and/or treatment of a wide range of diseases as disclosed herein above.

### Systems of Cells and Host Cells

The invention further relates to a system of cells, or a host cell, comprising a polynucleotide and/or a system as disclosed herein. The polynucleotide and/or system may have a sequence that is codon-optimised. Codon optimisation methods are known in the art and allow optimised expression in a heterologous host organism or cell system. In an embodiment the cell of the cell system may be selected from the group comprising bacteria, yeast, fungi, plant, mammalian and/or insect cells.

Methods for expressing a first polypeptide, a second polypeptide and/or a third polypeptide in a system of cells, or a host cell, are known in the art. The first, second or third polypeptide may be heterologously expressed from corresponding polynucleotide sequences cloned into the genome of cells of the cell system, or the host cell, or they may be comprised within a vector. For example, a first, second and/or third polynucleotide coding for the first, second and/or third polypeptide is cloned into the genome, and a first, second and/or third polynucleotide coding for the first, second and/or third polypeptide is comprised within a vector transformed or transfected into the system of cells, or host cell.

Expression of the first, second and third polypeptides in the cell may occur in a transient manner. When the polynucleotide encoding one of the polypeptides is cloned into the genome, an inducible promoter may be cloned as well to control expression of the polypeptides. Such inducible promoters are known in the art. Alternatively, genes coding for suppressors of gene silencing may also be cloned into the genome or into a vector transfected within the cell.

Also provided herein is thus a system of cells, or a host cell, expressing:
i) a particle-forming protein fused to a first peptide tag and, optionally, to a third peptide tag, optionally by a linker;
ii) an antigen fused to a second peptide tag, optionally by a linker; and
iii) at least one immune response modulating moiety (IRMM),
   wherein
a) i), ii) and iii) are in the form of one or more polypeptides or one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond or by affinity conjugation, optionally by a linker; or
   ii. is fused to a fourth peptide tag, optionally by a linker; and
c) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
      whereby i)-ii)-iii) form a particle displaying said antigen and said at least one IRMM.

Also provided herein is a system of cells, or a host cell, expressing:
i) a particle-forming protein fused to an antigen, optionally by a linker, and, optionally, to a third peptide tag, optionally by a linker; and
ii) at least one immune response modulating moiety (IRMM)
   wherein
a) i) and ii) are in the form of one or more polypeptides or one or more polynucleotides encoding i) and ii);
b) said at least one IRMM
   i) is linked to said particle-forming protein or said antigen by fusion, by an isopeptide bond, by an ester bond or by affinity conjugation, optionally by a linker; or
   ii) is fused to a fourth peptide tag, optionally by a linker; and
c) the at least one IRMM and the particle-forming protein are capable of being linked via an isopeptide bond, via an ester bond, via chemical crosslinking or via affinity conjugation between the third and the fourth peptide tags when present,
   whereby i)-ii) form a particle displaying said antigen and said at least one IRMM.

In some embodiments, the system of cells comprises different cells, wherein each cell comprises a part of the system as disclosed herein above. Thus, in some embodiments, the system of cells comprises cells comprising the first, the second, or the third polynucleotide of the system as disclosed herein above

In some embodiments, the system of cells, or the host cell, is one or more bacterial cell. In some embodiments, the system of cells, or the host cell, is a yeast cell. In some embodiments, the system of cells, or the host cell, is a fungal cell. In some embodiments, the system of cells, or the host cell, is a plant cell. In some embodiments, the system of cells, or the host cell, is a mammalian cell, such as a human cell. In some embodiments, the system of cells, or the host cell, is an insect cell.

In a particular embodiment the system of cells, or the host cell, may be one or more cells selected from the group comprising Escherichia coli, Spodoptera frugiperda (sf9), Trichoplusia ni (BTI-TN-5B1-4), Pichia Pastoris, Saccharomyces cerevisiae, Hansenula polymorpha, Drosophila Schneider 2 (S2), Lactococcus lactis, Chinese hamster ovary (CHO), Human Embryonic Kidney 293, Nicotiana tabacum cv. Samsun NN and Solanum tuberosum cv. Solara. Thus in an embodiment, the system of cells, or the host cell, is one or more cells of Escherichia coli. In another embodiment, the system of cells, or the host cell, is one or more cells of Spodoptera frugiperda. In another embodiment, the system of cells, or the host cell, is one or more cells of Pichia Pastoris. In another embodiment, the system of cells, or the host cell, is one or more cells of Saccharomyces cerevisiae. In another embodiment, the system of cells, or the host cell, is one or more cells of Hansenula polymorpha. In another embodiment, the system of cells, or the host cell, is one or more cells of Drosophila Schneider 2. In another embodiment, the system of cells, or the host cell, is one or more cells of Lactococcus lactis. In another embodiment, the system of cells, or the host cell, is one or more cells of Chinese hamster ovary (CHO). In another embodiment, the system of cells, or the host cell, is one or more cells of Human Embryonic Kidney 293. In another embodiment, the system of cells, or the host cell, is one or more cells of Trichoplusia ni (BTI-TN-5B1-4). In another embodiment, the system of cells, or the host cell, is one or more cells of Nicotiana tabacum cv. Samsun NN. In another embodiment, the system of cells, or the host cell, is one or more cells of Solanum tuberosum cv. Solara.

The present system of cells, or the host cells, can be used for prophylaxis and/or treatment of a wide range of diseases as disclosed herein above.

### Method for Modulating an Immune Response of a Subject to an Antigen

Also provided herein is a method of modulating an immune response of a subject to an antigen, the method comprising a step of administering a composition as described herein above at least once to a subject.

Thus, in some aspects is provided a method of modulating an immune response of a subject to an antigen, the method comprising a step of administering a composition to said subject at least once, wherein the composition comprises:
i) a particle-forming protein fused to a first peptide tag and, optionally, to a third peptide tag, optionally by a linker;
ii) said antigen fused to a second peptide tag, optionally by a linker; and
iii) at least one immune response modulating moiety (IRMM),
   wherein
a) i), ii) and iii) are provided as one or more polypeptides or as one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond or by affinity conjugation, optionally by a linker; or
   ii. is fused to a fourth peptide tag, optionally by a linker; and
c) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
      whereby i)-ii)-iii) form a particle displaying said antigen and said at least one IRMM.

In some aspects is provided a method of modulating an immune response of a subject to an antigen, the method comprising a step of administering a composition to said subject at least once, wherein the composition comprises:
i) a particle-forming protein fused to said antigen, optionally by a linker, and, optionally, to a third peptide tag, optionally by a linker; and
ii) at least one immune response modulating moiety (IRMM)
   wherein
a) i) and ii) are provided as a one or more polypeptides or as one or more polynucleotides encoding i) and ii);
b) said at least one IRMM
   i) is linked to said particle-forming protein or said antigen by fusion, by an isopeptide bond, by an ester bond or by affinity conjugation, optionally by a linker; or
   ii) is fused to a fourth peptide tag, optionally by a linker; and
c) the at least one IRMM and the particle-forming protein are capable of being linked via an isopeptide bond, via an ester bond, via chemical crosslinking or via affinity conjugation between the third and the fourth peptide tags when present,
   whereby i)-ii) form a particle displaying said antigen and said at least one IRMM.

Similarly, the present disclosure also provides the system as disclosed elsewhere herein, and/or the cell or host cell as disclosed elsewhere herein, for use in a method for inducing an immune response in a subject, the method comprising the step of administering said composition, polynucleotide or system to said subject at least once.

In some embodiments, said subject is a mammal. In some embodiments, said mammal is a human.

If it is desired to direct the immune response towards greater induction of total IgG as well as IgG2a, and without being bound by theory, then an IRMM comprising or consisting of a TLR4 agonist peptide, such as SEQ ID NO: 121, may advantageously be considered.

If it is desired to direct the immune response towards greater induction of IgG2a, IgG2b and IgG3, and without being bound by theory, then an IRMM comprising or consisting of a C3d complement peptide, such as SEQ ID NO: 125, may advantageously be considered.

If it is desired to direct the immune response towards lower induction of IgG1 and higher induction of IgG2a, IgG2b and IgG3, and without being bound by theory, then an IRMM comprising or consisting of a CD180 agonist peptide complement peptide, such as SEQ ID NO: 123, may advantageously be considered.

If it is desired to direct the immune response towards greater induction of total IgG, and without being bound by theory, then an IRMM comprising or consisting of a recombinant Flagellin, such as SEQ ID NO: 141, may advantageously be considered.

### Kit of Parts

In some aspects of the inventions is also provided a kit of parts comprising
i) a composition as defined elsewhere herein or a system as defined elsewhere herein, and
ii) optionally, a medical instrument or other means for administering the composition, and
iii) instructions for use.

In some embodiments, the kit of parts comprises a second active ingredient.

### EXAMPLES

### Example 1—TLR4 Agonist Peptides

The effect on vaccine-induced antibody responses after vaccination with antigen-displaying VLPs genetically modified by two different Toll-like receptor 4 (TLR4) agonist peptides was tested in mice.

### Method:

Two different TLR4 agonist peptides and a control peptide were designed.

| | |
|---|---|
| 1. | SpyCatcher-ggsgs-AP205-ggsgs-RYETMSIMIKSGGKY |
| 2. | SpyCatcher-ggsgs-AP205-ggsgs-APPHALS |
| 3. | SpyC-AP205 |

Balb/C mice were immunized using a two-week interval prime-boost regimen of with 2 μg modified (i.e. genetic fusion of TLR4 agonist peptide) or unmodified (i.e. control SpyC-AP205) VLPs displaying the SpyCatcher model antigen.

Serum was obtained two weeks after the last immunization and the total antigen-specific (i.e. anti-SpyCatcher (SpyC)) IgG including individual isotypes (IgG1, IgG2a, IgG2b and IgG3) was hereafter measured by ELISA.

### Results

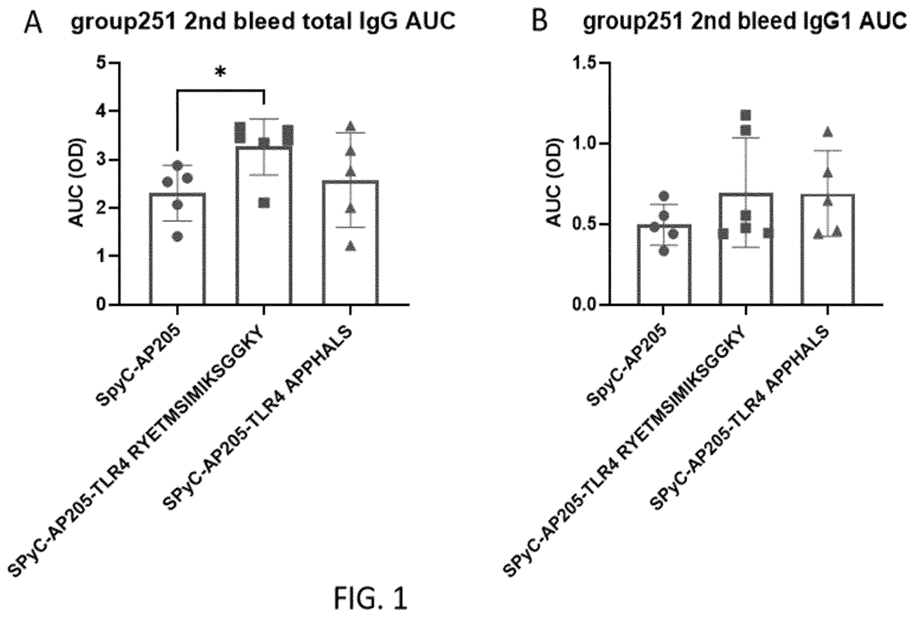

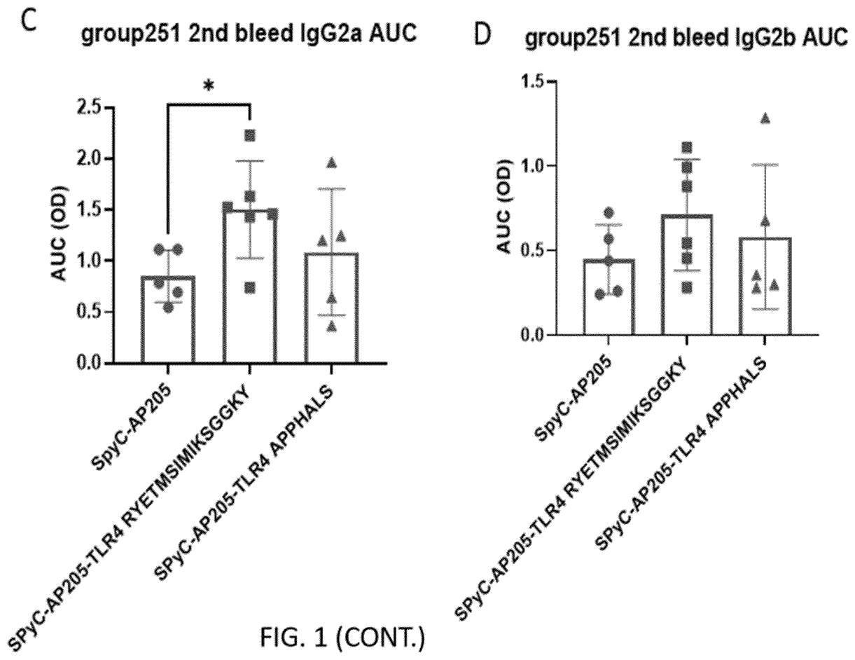

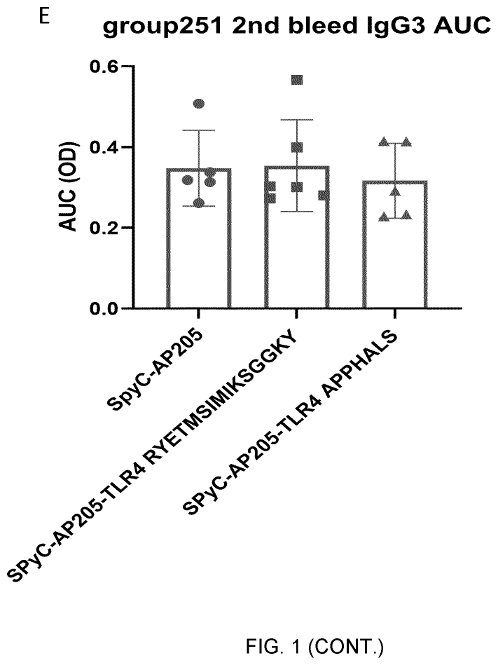

After two immunizations the modified SpyC-AP205-TLR4 (RYETMSIMIKSGGKY) VLP elicits significantly higher total anti-SpyCatcher IgG as well as IgG2a (FIG. 1 A and C).

Thus, the data show that fusion of an agonist of TLR4 to SpyC-AP205 leads to an increased anti-SpyCatcher antibody response with an altered isotype profile, i.e. more IgG2.

### Example 2—CD180 Agonist/C3d Complement Peptide

The effect on immune responses after vaccination with antigen-displaying VLPs genetically modified by a CD180 agonist peptide or a C3d complement peptide was tested in this example.

### Method

Three different constructs were designed.

| | |
|---|---|
| 1. | AP205 + Mc-RBD (Control vaccine) |
| 2. | aCD180-AP205-RT+ MC-RBD: (CSSNKSTTGSGETTTA- |
| | GSGTAGGGSGS-AP205-GTASGGSGGS-RumtrunkD9N) |
| 3. | RT-VLP-C3d + MC-RBD: (RumtrunkD9N-GSGTAGGGSGS- |
| | AP205-GTASGGSGGS-KFLNTAKDRNRWEEPDQQLYNVEATSYA) |

The antigen was conjugated to the VLP using genetically fused split-protein Tag/Catcher binding partners.

Balb/C mice were immunized with 2 μg modified (i.e. by genetic fusion of either CD180 agonist or C3d complement peptide) or unmodified AP205 VLPs displaying the receptor binding domain of the SARS-CoV-2 Spike protein. Serum was obtained two weeks after immunization.

The total antigen-specific (i.e. anti-SARS-CoV-2 Spike RBD) IgG including individual isotypes (IgG1, IgG2a, IgG2b and IgG3) was hereafter measured by ELISA.

### Results

### VLP-C3d

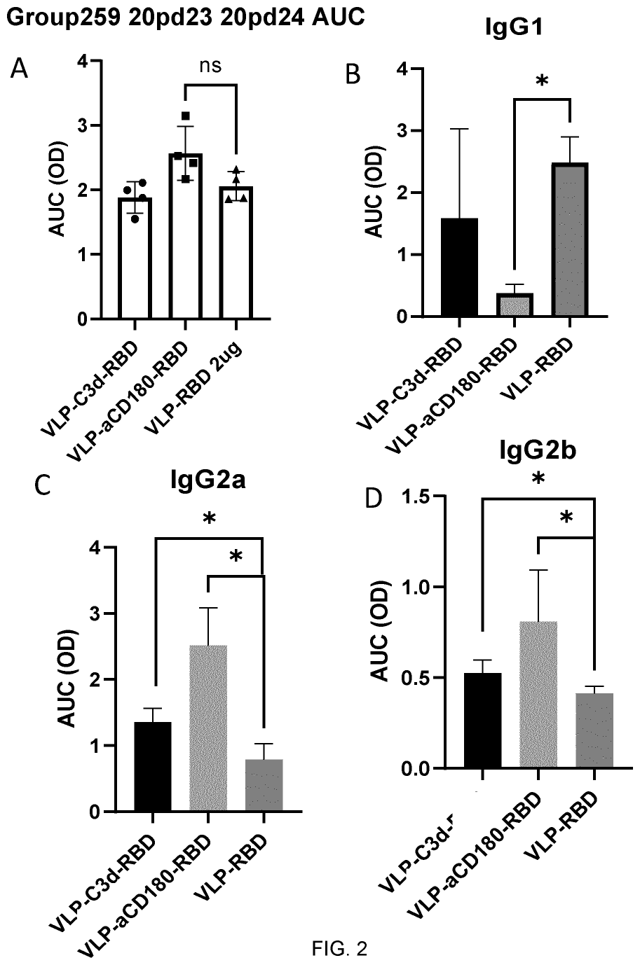

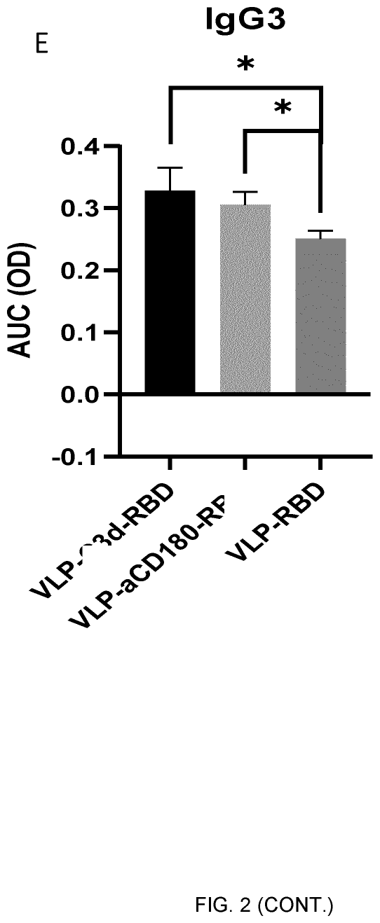

After one immunization, the anti-RBD Ab response induced by the modified VLP-C3d (i.e. genetically fused to the C3d complement peptide) has a different IgG isotype profile compared to the Ab response elicited by the unmodified VLP displaying the same RBD antigen. Specifically, the VLP-C3d induces comparatively higher levels of IgG2a, IgG2b and IgG3 (FIG. 2C-E).

### VLP-aCD180

After one immunization the anti-RBD Ab response induced by the modified VLP-aCD180 (i.e. genetically fused to the CD180 agonist peptide) has a different IgG isotype profile compared to the Ab response elicited by the unmodified VLP displaying the same RBD antigen. Specifically, the VLP-aCD180 induces comparatively lower level of IgG1 (FIG. 2E) and higher levels of IgG2a, IgG2b and IgG3 (FIG. 2C-E).

Overall, these results show that both VLP-C3d and VLP-aCD180 display a different isotype profile compared to the unmodified VLP displaying the same RBD antigen.

### Example 3—Flagellin

The AP205 VLP platform was employed to test the effect on immunogenicity of adding/displaying a recombinant Flagellin (Salmonella) on the AP205 VLP (conjugated via SpyTag/Catcher).

### Methods

Two different constructs were designed:
1. Flagellin-AP205-L2 VLP
2. AP205-1L2 VLP

Two groups with 6 mice in each (n=6) were immunized with 2 μg Flagellin-AP205-L2 VLPs or AP205-2 VLPs, respectively. Mice were immunized once and serum was collected two weeks after the immunization.

### Results

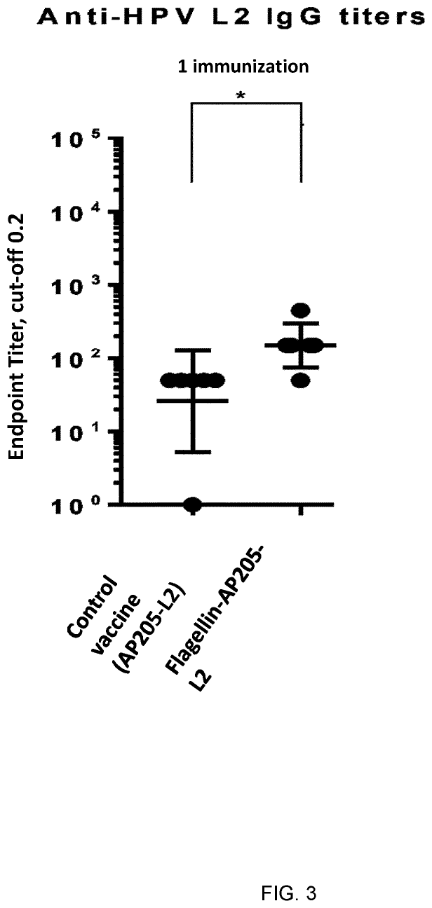

The in vivo immunization showed that VLPs co-displaying Flagellin stimulate a higher antigen-specific IgG response after 1 immunization compared to the control vaccine comprising a model antigen (HPV L2) (FIG. 3).

Sequence overview

| Sequence | | | |
|---|---|---|---|
| ID NO: | Organism | Name | Sequence |
| 1 | Streptococcus | SpyTag | AHIVMVDAYKPTK |
| | pyogenes | | |
| 2 | Streptococcus | SdyTag | DPIVMIDNDKPIT |
| | dysgalactiae | | |
| 3 | Streptococcus | SnoopTag | KLGDIEFIKVNK |
| | pneumoniae | | |
| 4 | Streptococcus | PhoTag (LPXTG | LVTGTAHIVMVDNYKPIVETGD |
| | phocae | cell wall anchor | |
| | | domain- | |
| | | containing | |
| | | protein)(NCBI | |
| | | Reference | |
| | | Sequence: | |
| | | WP_082385550.1) | |
| 5 | Enterococcus | EntTag | NTIVMVDKLKEVPPT |
| | faecalis | (hypothetical | |
| | | protein | |
| | | CUN42_14770, | |
| | | partial, GenBank | |
| | | PQC83400.1) | |
| 6 | Ruminococcus | RumTag (Cna B- | SENGNPLIVMVDDTTKVKIS |
| | sp. AF26-25AA | type domain- | |
| | | containing | |
| | | protein) | |
| 7 | Ruminococcus | Rum2Tag | GTPIVIMVDEAKPSLPD |
| | sp. AF25-19 | (hypothetical | |
| | | protein | |
| | | DWY44_14000, | |
| | | partial) | |
| 8 | Ruminococcus | Rum3Tag (TonB- | GNPLIVMIDEAEQKEIP |
| | sp. Marseille- | dependent | |
| | P6503 | receptor) | |
| 9 | Ruminococcus | Rum4Tag | AGGIIVMKDNTTKVSIS |
| | sp. | (hypothetical | |
| | | protein)(NCBI | |
| | | HCW12338.1) | |
| 10 | Ruminococcus | Rum5Tag (Cna B- | GNPIVTMIDDATLVKIS |
| | flavefaciens | type domain- | |
| | | containing | |
| | | protein)(NCBI | |
| | | WP_051536060.1) | |
| 11 | Ruminococcus | Rum6Tag | GNSTITMVDDTTKVHIT |
| | sp. | | |
| 12 | Ruminococcus | Rum7Tag (Cna B | GTPLIVMVDDTTKVEIS |
| | sp. AM43-6 | domain protein) | |
| | | (NCBI | |
| | | WP_118125159.1) | |
| 13 | Artificial | Rumtrunk D9N | GNPLIVMVNDTTKVK |
| | sequence | | |
| 14 | Ruminococcus | Rumtrunk tag | GNPLIVMVDDTTKVK |
| | sp. | | |
| 15 | Bacillus cereus | BacTag (choice- | NEKVTGQFEIVKVDANDKTK |
| | | of-anchor A family | |
| | | protein)(NCBI | |
| | | WP_080470427.1) | |
| 16 | Bacillus cereus | Bac2Tag | SKSLGQFEIVKVDAQDKTK |
| | | (hypothetical | |
| | | protein | |
| | | COD21_31890, | |
| | | partial)(NCBI | |
| | | PGT97799.1) | |
| 17 | Bacillus cereus | Bac3Tag (choice- | LGQFEIVKVDSQDKTK |
| | | of-anchor A family | |
| | | protein)(NCBI | |
| | | WP_053565148.1) | |
| 18 | Bacillus cereus | Bac4Tag (choice- | VTGQFEIVKVDAEDKTR |
| | | of-anchor A family | |
| | | protein) | |
| | | (WP_088344882. | |
| | | 1) | |
| 19 | Bacillus cereus | Bac5Tag | EKVMGQFEIMKVDANDKTK |
| | VD022 | (hypothetical | |
| | | protein | |
| | | IC1_02949, | |
| | | partial, | |
| | | EJP89589.1) | |
| 20 | Clostridium | Cpe0147 | DTKQVVKHEDKNDKAQTLVVEKP |
| | perfringens B | (Uniprot: B1R775) | |
| | str. ATCC 3626 | aa 565-587 | |
| | | (esterbond | |
| | | forming TAG) | |
| 21 | Streptococcus | SpyCatcher | GAMVDTLSGLSSEQGQSGDMTIEEDSAT |
| | pyogenes | | HIKFSKRDEDGKELAGATMELRDSSGKT |
| | | | ISTWISDGQVKDFYLYPGKYTFVETAAP |
| | | | DGYEVATAITFTVNEQGQVTVNGKATKG |
| | | | DAHI |
| 22 | Streptococcus | SdyCatcher | IDTMSGLSGETGQSGNTTIEEDSTTHVK |
| | dysgalactiae | | FSKRDSNGKELAGAMIELRNLSGQTIQS |
| | | | WVSDGTVKDFYLMPGTYQFVETAAPEGY |
| | | | ELAAPITFTIDEKGQIWVDS |
| 23 | Streptococcus | SnoopCatcher | SSGLVPRGSHMKPLRGAVFSLQKQHPDY |
| | pneumoniae | | PDIYGAIDQNGTYQNVRTGEDGKLTFKN |
| | | | LSDGKYRLFENSEPAGYKPVQNKPIVAF |
| | | | QIVNGEVRDVTSIVPQDIPATYEFTNGK |
| | | | HYITNEPIPPK |
| 24 | Actinomyces | FimP domain 3 | GSLSKYGKVILTKTGTDDLADKTKYNGA |
| | viscosus | | QFQVYECTKTASGATLRDSDPSTQTVDP |
| | | | LTIGGEKTFTTAGQGTVEINYLRANDYV |
| | | | NGAKKDQLTDEDYYCLVETKAPEGYNLQ |
| | | | ADPLPERVLAEKAEKKA |
| 25 | Streptococcus | Streptococcal | GSTTKVKLIKVDQDHNRLEGVGFKLVSV |
| | pneumonia | ancillary pilin | ARDVSAAAVPLIGEYRYSSSGQVGRTLY |
| | serotype 4 | Domain 2 | TDKNGEIFVTNLPLGNYRFKEVEPLAGY |
| | (strain ATCC | | AVTTLDTDVQLVDHQLVT |
| | BAA- | | |
| | 334/TIGR4) | | |
| 26 | Streptococcus | Streptococcal | PRGNVDFMKVDGRTNTSLQGAMFKVMKE |
| | pneumonia | ancillary pilin | ESGHYTPVLQNGKEVVVTSGKDGRFRVE |
| | serotype 4 | Domain 3 | GLEYGTYYLWELQAPTGYVQLTSPVSFT |
| | (strain ATCC | | IGKDTRKELV |
| | BAA- | | |
| | 334/TIGR4) | | |
| 27 | Corynebacterium | Major Pilin SpaD | VVTYHGKLKVVKKDGKEAGKVLKGAEFE |
| | diphtheriae | Domain 3 | LYQCTSAAVLGKGPLTVDGVKKWTTGDD |
| | NCTC 13129 | | GTFTIDGLHVTDFEDGKEAAPATKKFCL |
| | | | KETKAPAGYALPDPNVTEIEFTRAKISE |
| | | | KDKFEGDDEVT |
| 28 | Lactobacillus | Pilin subunit | STNDTTTQNVVLTKYGEDKDVTAIDRAT |
| | rhamnosus GG | (SpaA) domain 2 | DQIWTGDGAKPLQGVDFTIYNVTANYWA |
| | | | SPKDYKGSFDSAPVAATGTTNDKGQLTQ |
| | | | ALPIQSKDASGKTRAAVYLFHETNPRAG |
| | | | YNTSADFWLTLPAKAAADGNVY |
| 29 | Lactobacillus | Pilin subunit | TTYERTFVKKDAETKEVLEGAGFKISNS |
| | rhamnosus GG | (SpaA) domain 3 | DGKFLKLTDKDGQSVSIGEGFIDVLANN |
| | | | YRLTWVAESDATVFTSDKSGKFGLNGFA |
| | | | DNTTTYTAVETNVPDGYDAAANTDFKAD |
| | | | NS |
| 30 | Streptococcus | Surface protein | GQITIKKIDGSTKASLQGAIFVLKNATG |
| | agalactiae A909 | Spb1 domain 3 | QFLNFNDTNNVEWGTEANATEYTTGADG |
| | | | IITITGLKEGTYYLVEKKAPLGYNLLDN |
| | | | SQKVILGDGATDTTNSDNLLVNP |
| 31 | Streptococcus | PsCsCatcher | EQDVVFSKVNVAGEEIAGAKIQLKDAQG |
| | intermedius | (LPXTG cell wall | QVVHSWTSKAGQSETVKLKAGTYTFHEA |
| | | anchor domain- | SAPTGYLAVTDITFEVDVQGKVTVKDAN |
| | | containing | GNGVKAD |
| | | protein) | |
| 32 | Streptococcus | RgA Catcher | KLGDIEFIKVNKNDKKPLRGAVFSLQKQ |
| | pneumoniae | | HPDYPDIYGAIDQNGTYQNVRTGEDGKL |
| | | | TFKNLSDGKYRLFENSEPAGYKPVQNKP |
| | | | IVAFQIVNGEVRDVTSIVPQ |
| 33 | Corynebacterium | Major Pilin SpaD | GSERKGSLTLHKKKGAESEKRATGKEMD |
| | diphtheriae | Domain 1 | DVAGEPLNGVTFKITKLNFDLQNGDWAK |
| | NCTC 13129 | | FPKTAADAKGHETSTTKEVETSGNGTAV |
| | | | FDNLDLGIYLVEETKAPDGIVTGAPFIV |
| | | | SIPMVNEASDAWNYNVVA |
| 34 | Clostridium | Cpe0147 | NLPEVKDGTLRTTVIADGVNGSSEKEAL |
| | perfringens B | (Uniprot: B1R775) | VSFENSKDGVDVKDTINYEGLVANQNYT |
| | str. ATCC 3626 | aa 439-587 | LTGTLMHVKADGSLEEIATKTTNVTAGE |
| | | (esterbond | NGNGTWGLDFGNQKLQVGEKYVVFENAE |
| | | forming | SVENLIDTDKDYNLDTKQVVKHEDKNDK |
| | | CATCHER) | AQTLVVEKP |
| 35 | Streptococcus | SpyTag DNA | GCTCACATCGTGATGGTGGACGCTTACA |
| | pyogenes | | AGCCCACCAAG |
| 36 | Streptococcus | SdyTag DNA | GACCCCATCGTGATGATCGACAACGACA |
| | dysgalactiae | | AGCCCATCACC |
| 37 | Streptococcus | SnoopTag DNA | AAACTGGGTGATATTGAATTTATTAAAG |
| | pneumoniae | | TTAATAAA |
| 38 | Streptococcus | PhoTag DNA | CTGGTTACCGGCACCGCACATATTGTTA |
| | phocae | | TGGTTGATAACTATAAGCCGATCGTGGA |
| | | | AACCGGTGAT |
| 39 | Enterococcus | EntTag DNA | ACCGAAGTTAGCGGTAATACCATTGTGA |
| | faecalis | | TGGTGGATAAACTGAAAGAAGTTCCGCC |
| | | | TACC |
| 40 | Ruminococcus | RumTag DNA | AGCGAAAACGGCAACCCGCTGATTGTGA |
| | sp. AF26-25AA | | TGGTGGATGATACCACCAAAGTGAAAAT |
| | | | TAGC |
| 41 | Ruminococcus | Rum2Tag DNA | GGCACCCCGATTGTGATTATGGTGGATG |
| | sp. AF25-19 | | AAGCGAAACCGAGCCTGCCGGAT |
| 42 | Ruminococcus | Rum3Tag DNA | GGCAACCCGCTGATTGTGATGATTGATG |
| | sp. Marseille- | | AAGCGGAACAGAAAGAAATTCCG |
| | P6503 | | |
| 43 | Ruminococcus | Rum4Tag DNA | GCGGGCGGCATTATTGTGATGAAAGATA |
| | sp. | | ACACCACCAAAGTGAGCATTAGC |
| 44 | Ruminococcus | Rum5Tag DNA | GGCAACCCGATTGTGACCATGATTGATG |
| | flavefaciens | | ATGCGACCCTGGTGAAAATT |
| 45 | Ruminococcus | Rum6Tag DNA | GGCAACAGCACCATTACCATGGTGGATG |
| | sp. | | ATACCACCAAAGTGCATATTACC |
| 46 | Ruminococcus | Rum7Tag DNA | GGCACCCCGCTGATTGTGATGGTGGATG |
| | sp. AM43-6 | | ATACCACCAAAGTGGAAATTAGC |
| 47 | Artificial | Rumtrunk D9N | GGTAATCCGCTGATTGTGATGGTGAATG |
| | sequence | DNA | ATACCACCAAAGTGAAA |
| 48 | Ruminococcus | Rumtrunk tag | GGCAACCCGCTGATTGTGATGGTGGATG |
| | sp. | | ATACCACCAAAGTGAAA |
| 49 | Bacillus cereus | BacTag DNA | GGTCAGTTCGAAATTGTTAAAGTTGATG |
| | | | CAAACGATAAAACTAAA |
| 50 | Bacillus cereus | Bac2Tag DNA | AGCAAAAGCCTGGGCCAGTTTGAAATTG |
| | | | TGAAAGTGGATGCGCAGGATAAAACCAA |
| | | | A |
| 51 | Bacillus cereus | Bac3Tag DNA | CTGGGCCAGTTTGAAATTGTTAAAGTTG |
| | | | ATAGCCAGGATAAAACCAAA |
| 52 | Bacillus cereus | Bac4Tag DNA | GTTACCGGTCAGTTTGAAATCGTTAAAG |
| | | | TTGATGCCGAAGATAAGACCCGT |
| 53 | Bacillus cereus | Bac5Tag DNA | GAAAAAGTGATGGGCCAGTTCGAAATCA |
| | VD022 | | TGAAAGTTGATGCCAACGACAAGACCAA |
| | | | A |
| 54 | Clostridium | cpe0147 (Uniprot: | GACACCAAGCAGGTGGTGAAGCACGAGG |
| | perfringens B | B1R775) aa 565- | ACAAGAACGACAAGGCCCAGACCCTGGT |
| | str. ATCC 3626 | 587 (esterbond | GGTGGAGAAGCCC |
| | | forming TAG) | |
| | | DNA | |
| 55 | Streptococcus | SpyCatcher DNA | GGTGCAATGGTTGATACCCTGAGCGGTC |
| | pyogenes | | TGAGCAGCGAACAGGGTCAGAGCGGTGA |
| | | | TATGACCATTGAAGAAGATAGCGCAACC |
| | | | CACATCAAATTCAGCAAACGTGATGAAG |
| | | | ATGGTAAAGAACTGGCAGGCGCAACAAT |
| | | | GGAACTGCGTGATAGCAGCGGTAAAACC |
| | | | ATTAGCACCTGGATTAGTGATGGTCAGG |
| | | | TGAAAGATTTTTATCTGTACCCTGGCAA |
| | | | ATACACCTTTGTTGAAACCGCAGCACCG |
| | | | GATGGTTATGAAGTTGCAACCGCAATTA |
| | | | CCTTTACCGTTAATGAACAGGGCCAGGT |
| | | | TACCGTGAATGGTAAAGCAACCAAAGGT |
| | | | GATGCACATATT |
| 56 | Streptococcus | SdyCatcher DNA | ATGGGTATTGATACCATGAGCGGTCTGA |
| | dysgalactiae | | GCGGTGAAACCGGTCAGAGCGGTAATAC |
| | | | CACCATTGAAGAGGATAGCACCACACAT |
| | | | GTGAAATTCAGCAAACGCGATGCAAACG |
| | | | GCAAAGAACTGGCAGGCGCAATGATTGA |
| | | | ACTGCGTAATCTGAGTGGTCAGACCATT |
| | | | CAGAGCTGGGTTAGTGATGGCACCGTTA |
| | | | AAGATTTTTATCTGATGCCTGGCACCTA |
| | | | TCAGTTTGTTGAAACCGCAGCACCGGAA |
| | | | GGTTATGAGCTGGCAGCACCGATTACCT |
| | | | TTACCATTGATGAAAAAGGTCAGATTTG |
| | | | GGTTGATAGC |
| 57 | Streptococcus | SnoopCatcher | AGCAGCGGCCTGGTGCCGCGCGGCAGCC |
| | pneumoniae | DNA | ATATGAAGCCGCTGCGTGGTGCCGTGTT |
| | | | TAGCCTGCAGAAACAGCATCCCGACTAT |
| | | | CCCGATATCTATGGCGCGATTGATCAGA |
| | | | ATGGGACCTATCAAAATGTGCGTACCGG |
| | | | CGAAGATGGTAAACTGACCTTTAAGAAT |
| | | | CTGAGCGATGGCAAATATCGCCTGTTTG |
| | | | AAAATAGCGAACCCGCTGGCTATAAACC |
| | | | GGTGCAGAATAAGCCGATTGTGGCGTTT |
| | | | CAGATTGTGAATGGCGAAGTGCGTGATG |
| | | | TGACCAGCATTGTGCCGCAGGATATTCC |
| | | | GGCTACATATGAATTTACCAACGGTAAA |
| | | | CATTATATCACCAATGAACCGATACCGC |
| | | | CGAAA |
| 58 | Actinomyces | FimP domain 3 | GGTAGCCTGAGCAAATATGGTAAAGTGA |
| | viscosus | DNA | TTCTGACCAAAACCGGCACCGATGATCT |
| | | | GGCAGATAAAACCAAATATAACGGTGCA |
| | | | CAGTTTCAGGTGTATGAATGTACCAAAA |
| | | | CAGCAAGCGGTGCAACCCTGCGTGATAG |
| | | | CGATCCGAGCACACAGACCGTTGATCCG |
| | | | CTGACCATTGGTGGTGAAAAAACCTTTA |
| | | | CCACCGCAGGTCAGGGCACCGTTGAAAT |
| | | | TAATTATCTGCGTGCCAATGATTATGTG |
| | | | AACGGTGCAAAAAAAGATCAGCTGACCG |
| | | | ATGAAGATTATTACTGTCTGGTTGAAAC |
| | | | CAAAGCACCGGAAGGTTATAATCTGCAG |
| | | | GCAGATCCGCTGCCGTTTCGTGTTCTGG |
| | | | CCGAAAAAGCAGAAAAAAAAGCC |
| 59 | Streptococcus | Streptococcal | GGTAGCACCACCAAAGTGAAACTGATTA |
| | pneumonia | ancillary pilin | AAGTTGATCAGGATCACAATCGTCTGGA |
| | serotype 4 | Domain 2 DNA | AGGTGTTGGTTTTAAACTGGTTAGCGTT |
| | (strain ATCC | | GCACGTGATGTTAGCGCAGCAGCAGTTC |
| | BAA- | | CGCTGATTGGTGAATATCGTTATAGCAG |
| | 334/TIGR4) | | CAGCGGTCAGGTTGGTCGTACCCTGTAT |
| | | | ACCGATAAAAATGGCGAAATTTTCGTTA |
| | | | CCAATCTGCCGCTGGGTAACTATCGTTT |
| | | | TAAAGAAGTTGAACCGCTGGCAGGTTAT |
| | | | GCAGTTACCACACTGGATACCGATGTTC |
| | | | AGCTGGTTGATCATCAGCTGGTGACC |
| 60 | Streptococcus | Streptococcal | CCGCGTGGTAATGTTGATTTTATGAAAG |
| | pneumonia | ancillary pilin | TTGATGGTCGCACCAATACCAGCCTGCA |
| | serotype 4 | Domain 3 DNA | GGGTGCAATGTTTAAAGTGATGAAAGAA |
| | (strain ATCC | | GAAAGCGGTCACTATACACCGGTGCTGC |
| | BAA- | | AGAATGGTAAAGAAGTTGTTGTTACCAG |
| | 334/TIGR4) | | CGGTAAAGATGGTCGTTTTCGTGTTGAA |
| | | | GGTCTGGAATATGGCACCTATTATCTGT |
| | | | GGGAACTGCAGGCACCGACCGGTTATGT |
| | | | TCAGCTGACCAGTCCGGTTAGTTTTACC |
| | | | ATTGGCAAAGATACCCGTAAAGAACTGG |
| | | | TG |
| 61 | Corynebacterium | Major Pilin SpaD | GTTGTTACCTATCATGGTAAACTGAAAG |
| | diphtheriae | Domain 3 DNA | TGGTGAAAAAAGACGGTAAAGAGGCAGG |
| | NCTC 13129 | | CAAAGTTCTGAAAGGTGCAGAATTTGAA |
| | | | CTGTATCAGTGTACCAGCGCAGCAGTTT |
| | | | TAGGTAAAGGTCCGCTGACCGTTGATGG |
| | | | TGTGAAAAAATGGACCACCGGTGATGAT |
| | | | GGCACCTTTACCATTGATGGTCTGCATG |
| | | | TTACCGATTTTGAAGATGGTAAAGAAGC |
| | | | CGCACCGGCAACCAAAAAATTCTGTCTG |
| | | | AAAGAAACCAAAGCACCGGCAGGTTATG |
| | | | CACTGCCTGATCCGAATGTGACCGAAAT |
| | | | TGAATTTACCCGTGCAAAAATCAGCGAG |
| | | | AAAGATAAATTTGAAGGCGACGATGAAG |
| | | | TGACC |
| 62 | Lactobacillus | Pilin subunit | AGCACCAATGATACCACCACACAGAATG |
| | rhamnosus GG | (SpaA) domain 2 | TTGTTCTGACCAAATATGGCTTCGATAA |
| | | DNA | AGATGTTACCGCAATTGATCGTGCAACC |
| | | | GATCAGATTTGGACCGGTGATGGTGCAA |
| | | | AACCGCTGCAGGGTGTTGATTTTACCAT |
| | | | TTATAACGTGACCGCCAATTATTGGGCA |
| | | | AGCCCGAAAGATTATAAAGGCAGCTTTG |
| | | | ATAGCGCACCGGTTGCAGCCACCGGTAC |
| | | | AACAAATGATAAAGGCCAGCTGACCCAG |
| | | | GCACTGCCGATTCAGAGCAAAGATGCAA |
| | | | GCGGTAAAACCCGTGCAGCAGTTTACCT |
| | | | GTTTCACGAAACCAATCCGCGTGCAGGT |
| | | | TATAATACCAGCGCAGATTTTTGGCTGA |
| | | | CCCTGCCTGCAAAAGCAGCAGCAGATGG |
| | | | TAATGTTTAT |
| 63 | Lactobacillus | Pilin subunit | ACCACCTATGAACGTACCTTTGTTAAAA |
| | rhamnosus GG | (SpaA) domain 3 | AAGACGCCGAAACCAAAGAAGTTCTGGA |
| | | DNA | AGGCGCAGGCTTTAAAATCAGCAATAGT |
| | | | GATGGCAAATTCCTGAAACTGACCGATA |
| | | | AAGATGGTCAGAGCGTTAGCATTGGTGA |
| | | | AGGTTTTATTGATGTTCTGGCCAATAAC |
| | | | TATCGTCTGACCTGGGTTGCAGAAAGTG |
| | | | ATGCAACCGTTTTTACCAGCGATAAAAG |
| | | | CGGCAAATTTGGTCTGAATGGTTTTGCA |
| | | | GATAATACCACCACCTATACCGCAGTTG |
| | | | AAACCAATGTTCCGGATGGTTATGATGC |
| | | | AGCAGCAAACACCGATTTCAAAGCCGAT |
| | | | AATAGC |
| 64 | Streptococcus | Surface protein | GGTCAGATTACCATCAAAAAAATCGATG |
| | agalactiae A909 | Spb1 domain 3 | GTAGCACCAAAGCAAGCCTGCAGGGTGC |
| | | DNA | AATTTTTGTTCTGAAAAATGCAACCGGT |
| | | | CAGTTCCTGAATTTTAACGATACCAATA |
| | | | ATGTTGAATGGGGCACCGAAGCAAATGC |
| | | | CACCGAATATACCACCGGTGCAGATGGT |
| | | | ATTATTACCATTACCGGTCTGAAAGAAG |
| | | | GCACCTATTACCTGGTTGAAAAAAAAGC |
| | | | ACCGCTGGGTTATAATCTGCTGGATAAT |
| | | | TCACAGAAAGTGATTTTAGGTGATGGTG |
| | | | CAACCGATACCACCAATAGCGATAACCT |
| | | | GCTGGTTAATCCG |
| 65 | Streptococcus | PsCsCatcher | GAACAGGATGTTGTGTTTAGCAAAGTTA |
| | intermedius | (LPXTG cell wall | ATGTTGCCGGTGAAGAAATTGCGGGTGC |
| | | anchor domain- | AAAAATCCAGCTGAAAGATGCACAGGGT |
| | | containing | CAAGTTGTTCATAGCTGGACCAGCAAAG |
| | | protein) DNA | CAGGTCAGAGCGAAACCGTTAAACTGAA |
| | | | AGCAGGCACCTATACCTTTCATGAAGCA |
| | | | AGCGCACCGACCGGTTATCTGGCAGTTA |
| | | | CCGATATTACCTTTGAAGTTGATGTTCA |
| | | | GGGTAAAGTGACCGTTAAAGATGCAAAT |
| | | | GGTAATGGTGTGAAAGCCGAC |
| 66 | Streptococcus | RgA Catcher DNA | AAACTGGGTGATATTGAGTTCATCAAAG |
| | pneumoniae | | TGAACAAAAACGATAAAAAACCGCTGCG |
| | | | TGGTGCAGTTTTTAGCCTGCAGAAACAG |
| | | | CATCCGGATTACCCGGATATTTATGGTG |
| | | | CAATTGATCAGAATGGCACCTATCAGAA |
| | | | TGTTCGTACCGGTGAAGATGGTAAACTG |
| | | | ACCTTTAAAAACCTGAGCGACGGTAAAT |
| | | | ATCGCCTGTTTGAAAATAGCGAACCGGC |
| | | | AGGTTATAAACCGGTTCAGAATAAACCG |
| | | | ATTGTGGCCTTTCAGATTGTTAATGGTG |
| | | | AAGTTCGTGATGTGACCAGCATTGTTCC |
| | | | GCAG |
| 67 | Corynebacterium | Major Pilin SpaD | GGTAGCGAACGTAAAGGTAGTCTGACCC |
| | diphtheriae | Domain 1 DNA | TGCATAAAAAGAAAGGTGCAGAAAGCGA |
| | NCTC 13129 | | AAAACGTGCAACCGGTAAAGAAATGGAT |
| | | | GATGTTGCCGGTGAACCGCTGAATGGTG |
| | | | TTACCTTTAAAATCACCAAACTGAACTT |
| | | | CGATCTGCAGAATGGTGATTGGGCAAAA |
| | | | TTTCCGAAAACCGCAGCAGATGCAAAAG |
| | | | GTCATGAAACCAGCACCACCAAAGAAGT |
| | | | GGAAACCAGCGGTAATGGCACCGCAGTT |
| | | | TTTGATAATCTGGATCTGGGTATTTACC |
| | | | TGGTGGAAGAAACCAAAGCACCGGATGG |
| | | | TATTGTTACAGGTGCACCGTTTATTGTT |
| | | | AGCATTCCGATGGTTAATGAAGCAAGTG |
| | | | ATGCCTGGAATTATAACGTTGTTGCA |
| 68 | Clostridium | cpe0147(uniprot: | AACCTGCCCGAGGTGAAGGACGGCACCC |
| | perfringens B | B1R775) aa 439- | TGAGGACCACCGTGATCGCCGACGGCGT |
| | str. ATCC 3626 | 587 (esterbond | GAACGGCAGCAGCGAGAAGGAGGCCCTG |
| | | forming catcher) | GTGAGCTTCGAGAACAGCAAGGACGGCG |
| | | DNA | TGGACGTGAAGGACACCATCAACTACGA |
| | | | GGGCCTGGTGGCCAACCAGAACTACACC |
| | | | CTGACCGGCACCCTGATGCACGTGAAGG |
| | | | CCGACGGCAGCCTGGAGGAGATCGCCAC |
| | | | CAAGACCACCAACGTGACCGCCGGCGAG |
| | | | AACGGCAACGGCACCTGGGGCCTGGACT |
| | | | TCGGCAACCAGAAGCTGCAGGTGGGCGA |
| | | | GAAGTACGTGGTGTTCGAGAACGCCGAG |
| | | | AGCGTGGAGAACCTGATCGACACCGACA |
| | | | AGGACTACAACCTGGACACCAAGCAGGT |
| | | | GGTGAAGCACGAGGACAAGAACGACAAG |
| | | | GCCCAGACCCTGGTGGTGGAGAAGCCC |
| 69 | Homo sapiens | Ferritin, aa 5-174 | RMLKALNDQLNRELYSAYLYFAMAAYFE |
| | | | DLGLEGFANWMKAQAEEEIGHALRFYNY |
| | | | IYDRNGRVELDEIPKPPKEWESPLKAFE |
| | | | AAYEHEKFISKSIYELAALAEEEKDYST |
| | | | RAFLEWFINEQVEEEASVKKILDKLKFA |
| | | | KDSPQILFMLDKELSARAPKLPGLLMQG |
| | | | GE |
| 70 | Homo sapiens | Lumazine | MQIYEGKLTAEGLRFGIVASRFNHALVD |
| | | synthase | RLVEGAIDCIVRHGGREEDITLVRVPGS |
| | | | WEIPVAAGELARKEDIDAVIAIGVLIRG |
| | | | ATPHFDYIASEVSKGLANLSLELRKPIT |
| | | | FGVITADTLEQAIERAGTKHGNKGWEAA |
| | | | LSAIEMANLFKSLR |
| 71 | Homo sapiens | NSP10 | PANSTVLSFCAFAVDPAKAYKDYLASGG |
| | | | QPITNCVKMLCTHTGTGQAITVTPEANM |
| | | | DQESFGGASCCLYCRCHIDHPNPKGFCD |
| | | | LKGKYVQIPTTCANDPVGFTLRNTVCTV |
| | | | CGMWKGYGCS |
| 72 | Homo sapiens | 1301 | MKMEELFKKHKIVAVLRANSVEEAKKKA |
| | | | LAVFLGGVHLIEITFTVPDADTVIKELS |
| | | | FLKEMGAIIGAGTVTSVEQARKAVESGA |
| | | | EFIVSPHLDEEISQFAKEKGVFYMPGVM |
| | | | TPTELVKAMKLGHTILKLFPGEVVGPQF |
| | | | VKAMKGPFPNVKFVPTGGVNLDNVCEWF |
| | | | KAGVLAVGVGSALVKGTPVEVAEKAKAF |
| | | | VEKIRGCTE |
| 73 | Thermotoga | 5KP9 | MGARASGSKSGSGSDSGSKMEELFKKHK |
| | maritima | | IVAVLRANSVEEAKKKALAVFLGGVHLI |
| | | | EITFTVPDADTVIKELSFLKEMGAIIGA |
| | | | GTVTSVEQCRKAVESGAEFIVSPHLDEE |
| | | | ISQFCKEKGVFYMPGVMTPTELVKAMKL |
| | | | GHTILKLFPGEVVGPQFVKAMKGPFPNV |
| | | | KFVPTGGVNLDNVCEWFKAGVLAVGVGS |
| | | | ALVKGTPVEVAEKAKAFVEKIRGCTEQK |
| | | | LISEEDLQSRPEPTAPPEESFRSGVETT |
| | | | TPPQKQEPIDKELYPLTSLRSLFGNDPS |
| | | | SQ |
| 74 | Homo sapiens | Ferritin DNA | AGAATGCTGAAGGCCCTGAACGACCAGC |
| | | sequence | TGAACAGAGAGCTGTACTCCGCCTACCT |
| | | covering aa 5-174 | GTACTTCGCTATGGCCGCCTACTTCGAG |
| | | | GACCTGGGCCTTGAGGGATTCGCCAACT |
| | | | GGATGAAGGCTCAGGCCGAGGAAGAGAT |
| | | | CGGCCACGCTCTGAGATTCTACAACTAC |
| | | | ATCTACGACAGAAACGGCCGCGTGGAAC |
| | | | TGGACGAGATCCCCAAGCCTCCTAAAGA |
| | | | GTGGGAGAGCCCTCTGAAGGCTTTCGAG |
| | | | GCTGCTTACGAGCACGAGAAGTTCATCA |
| | | | GCAAGAGCATCTACGAGCTGGCCGCTCT |
| | | | GGCCGAAGAGGAAAAGGACTACTCTACC |
| | | | AGAGCCTTCCTGGAATGGTTCATCAACG |
| | | | AACAGGTGGAAGAGGAAGCCAGCGTCAA |
| | | | GAAGATCCTGGACAAGCTGAAGTTCGCC |
| | | | AAGGACAGCCCTCAGATCCTGTTCATGC |
| | | | TGGACAAAGAGCTGAGCGCCAGGGCTCC |
| | | | TAAACTGCCTGGACTGCTTATGCAAGGC |
| | | | GGCGAA |
| 75 | Homo sapiens | Lumazine | ATGCAGATCTACGAGGGCAAGCTGACCG |
| | | synthase DNA | CCGAGGGCCTGAGGTTCGGCATCGTGGC |
| | | | CAGCAGGTTCAACCACGCCCTGGTGGAC |
| | | | AGGCTGGTGGAGGGCGCCATCGACTGCA |
| | | | TCGTGAGGCACGGCGGCAGGGAGGAGGA |
| | | | CATCACCCTGGTGAGGGTGCCCGGCAGC |
| | | | TGGGAGATCCCCGTGGCCGCCGGCGAGC |
| | | | TGGCCAGGAAGGAGGACATCGACGCCGT |
| | | | GATCGCCATCGGCGTGCTGATCAGGGGC |
| | | | GCCACCCCCCACTTCGACTACATCGCCA |
| | | | GCGAGGTGAGCAAGGGCCTGGCCAACCT |
| | | | GAGCCTGGAGCTGAGGAAGCCCATCACC |
| | | | TTCGGCGTGATCACCGCCGACACCCTGG |
| | | | AGCAGGCCATCGAGAGGGCCGGCACCAA |
| | | | GCACGGCAACAAGGGCTGGGAGGCCGCC |
| | | | CTGAGCGCCATCGAGATGGCCAACCTGT |
| | | | TCAAGAGCCTGAGG |
| 76 | Homo sapiens | NSP10 DNA | CCCGCCAACAGCACCGTGCTGAGCTTCT |
| | | | GCGCCTTCGCCGTGGACCCCGCCAAGGC |
| | | | CTACAAGGACTACCTGGCCAGCGGCGGC |
| | | | CAGCCCATCACCAACTGCGTGAAGATGC |
| | | | TGTGCACCCACACCGGCACCGGCCAGGC |
| | | | CATCACCGTGACCCCCGAGGCCAACATG |
| | | | GACCAGGAGAGCTTCGGCGGCGCCAGCT |
| | | | GCTGCCTGTACTGCAGGTGCCACATCGA |
| | | | CCACCCCAACCCCAAGGGCTTCTGCGAC |
| | | | CTGAAGGGCAAGTACGTGCAGATCCCCA |
| | | | CCACCTGCGCCAACGACCCCGTGGGCTT |
| | | | CACCCTGAGGAACACCGTGTGCACCGTG |
| | | | TGCGGCATGTGGAAGGGCTACGGCTGCA |
| | | | GC |
| 77 | Homo sapiens | I301 DNA | ATGAAGATGGAGGAGCTGTTCAAGAAGC |
| | | sequence | ACAAGATCGTGGCCGTGCTGAGGGCCAA |
| | | (optimized for | CAGCGTGGAGGAGGCCAAGAAGAAGGCC |
| | | human | CTGGCCGTGTTCCTGGGCGGCGTGCACC |
| | | expression) | TGATCGAGATCACCTTCACCGTGCCCGA |
| | | | CGCCGACACCGTGATCAAGGAGCTGAGC |
| | | | TTCCTGAAGGAGATGGGCGCCATCATCG |
| | | | GCGCCGGCACCGTGACCAGCGTGGAGCA |
| | | | GGCCAGGAAGGCCGTGGAGAGCGGCGCC |
| | | | GAGTTCATCGTGAGCCCCCACCTGGACG |
| | | | AGGAGATCAGCCAGTTCGCCAAGGAGAA |
| | | | GGGCGTGTTCTACATGCCCGGCGTGATG |
| | | | ACCCCCACCGAGCTGGTGAAGGCCATGA |
| | | | AGCTGGGCCACACCATCCTGAAGCTGTT |
| | | | CCCCGGCGAGGTGGTGGGCCCCCAGTTC |
| | | | GTGAAGGCCATGAAGGGCCCCTTCCCCA |
| | | | ACGTGAAGTTCGTGCCCACCGGCGGCGT |
| | | | GAACCTGGACAACGTGTGCGAGTGGTTC |
| | | | AAGGCCGGCGTGCTGGCCGTGGGCGTGG |
| | | | GCAGCGCCCTGGTGAAGGGCACCCCCGT |
| | | | GGAGGTGGCCGAGAAGGCCAAGGCCTTC |
| | | | GTGGAGAAGATCAGGGGCTGCACCGAG |
| 78 | Thermotoga | 5KP9 DNA | ATGGGCGCCAGGGCCAGCGGCAGCAAGA |
| | maritima | | GCGGCAGCGGCAGCGACAGCGGCAGCAA |
| | | | GATGGAGGAGCTGTTCAAGAAGCACAAG |
| | | | ATCGTGGCCGTGCTGAGGGCCAACAGCG |
| | | | TGGAGGAGGCCAAGAAGAAGGCCCTGGC |
| | | | CGTGTTCCTGGGCGGCGTGCACCTGATC |
| | | | GAGATCACCTTCACCGTGCCCGACGCCG |
| | | | ACACCGTGATCAAGGAGCTGAGCTTCCT |
| | | | GAAGGAGATGGGCGCCATCATCGGCGCC |
| | | | GGCACCGTGACCAGCGTGGAGCAGTGCA |
| | | | GGAAGGCCGTGGAGAGCGGCGCCGAGTT |
| | | | CATCGTGAGCCCCCACCTGGACGAGGAG |
| | | | ATCAGCCAGTTCTGCAAGGAGAAGGGCG |
| | | | TGTTCTACATGCCCGGCGTGATGACCCC |
| | | | CACCGAGCTGGTGAAGGCCATGAAGCTG |
| | | | GGCCACACCATCCTGAAGCTGTTCCCCG |
| | | | GCGAGGTGGTGGGCCCCCAGTTCGTGAA |
| | | | GGCCATGAAGGGCCCCTTCCCCAACGTG |
| | | | AAGTTCGTGCCCACCGGCGGCGTGAACC |
| | | | TGGACAACGTGTGCGAGTGGTTCAAGGC |
| | | | CGGCGTGCTGGCCGTGGGCGTGGGCAGC |
| | | | GCCCTGGTGAAGGGCACCCCCGTGGAGG |
| | | | TGGCCGAGAAGGCCAAGGCCTTCGTGGA |
| | | | GAAGATCAGGGGCTGCACCGAGCAGAAG |
| | | | CTGATCAGCGAGGAGGACCTGCAGAGCA |
| | | | GGCCCGAGCCCACCGCCCCCCCCGAGGA |
| | | | GAGCTTCAGGAGCGGCGTGGAGACCACC |
| | | | ACCCCCCCCCAGAAGCAGGAGCCCATCG |
| | | | ACAAGGAGCTGTACCCCCTGACCAGCCT |
| | | | GAGGAGCCTGTTCGGCAACGACCCCAGC |
| | | | AGCCAG |
| 79 | Homo sapiens | SP of azurocidin | MTRLTVLALLAGLLASSRA |
| 80 | Homo sapiens | Serum albumin | MKWVTFISLLFLFSSAYS |
| 81 | Homo sapiens | Modified serum | MKWVTFISLLFLFSSSSRA |
| | | albumin | |
| 82 | Cricetulus | Ig kappa chain V | MGSAALLLWVLLLWVPGSNG |
| | griseus | Ill region | |
| 83 | Cricetulus | Modified Ig kappa | MGSAALLLWVLLLWVPSSRA |
| | griseus | chain V III region | |
| | | MOPC 63 like | |
| | | (mIgK C) | |
| 84 | Homo sapiens | Modified Ig kappa | MEAPAQLLFLLLLWLPSSRA |
| | | chain V III region | |
| | | VG (mIgK H) | |
| 85 | Artificial | pVax1_SpyTag- | AHIVMVDAYKPTKGSGTAGGGSGSANKP |
| | | AP205 (amino | MQPITSTANKIVWSDPTRLSTTFSASLL |
| | | acid) | RQRVKVGIAELNNVSGQYVSVYKRPAPK |
| | | | PEGCADACVIMPNENQSIRTVISGSAEN |
| | | | LATLKAEWETHKRNVDTLFASGNAGLGF |
| | | | LDPTAAIVSSDTTA |
| 86 | Artificial | pVAX1_HBc- | MDIDPYKEFGASVELLSFLPSDFFPSIR |
| | | SpyTag (amino | DLLDTASALYREALESPEHVSPHHTALR |
| | | acid) | QAILCWGELMNLATWVGSNLEDPASREL |
| | | | VVSYVNVNMGLKLRQILWFHISCLTFGR |
| | | | ETVLEYLVSFGVWIRTPTAYRPPNAPIL |
| | | | STLPETTVVGGGGGSPGGGTPSPGGGGS |
| | | | QSPGGGGSQSGESQCGSAHIVMVDAYKP |
| | | | TK |
| 87 | Artificial | pVAX1- | GAMVDTLSGLSSEQGQSGDMTIEEDSAT |
| | | SPYCEGFP | HIKFSKRDEDGKELAGATMELRDSSGKT |
| | | (amino acid) | ISTWISDGQVKDFYLYPGKYTFVETAAP |
| | | | DGYEVATAITFTVNEQGQVTVNGKATKG |
| | | | DAHIGGSGSMVSKGEELFTGVVPILVEL |
| | | | DGDVNGHKFSVSGEGEGDATYGKLTLKF |
| | | | ICTTGKLPVPWPTLVTTLTYGVQCFSRY |
| | | | PDHMKQHDFFKSAMPEGYVQERTIFFKD |
| | | | DGNYKTRAEVKFEGDTLVNRIELKGIDF |
| | | | KEDGNILGHKLEYNYNSHNVYIMADKQK |
| | | | NGIKVNFKIRHNIEDGSVQLADHYQQNT |
| | | | PIGDGPVLLPDNHYLSTQSALSKDPNEK |
| | | | RDHMVLLEFVTAAGITLGMDELYK |
| 88 | Artificial | pVax1_SpyTag- | GCTCACATCGTGATGGTGGACGCCTACA |
| | | AP205 (DNA) | AGCCCACAAAAGGCTCTGGAACAGCTGG |
| | | | CGGCGGATCTGGCTCTGCCAACAAACCT |
| | | | ATGCAGCCCATCACCAGCACCGCCAACA |
| | | | AGATCGTTTGGAGCGACCCCACCAGACT |
| | | | GAGCACCACATTCAGCGCTAGCCTGCTG |
| | | | AGACAGCGCGTGAAAGTGGGAATCGCCG |
| | | | AGCTGAACAATGTGTCCGGCCAGTACGT |
| | | | GTCCGTGTACAAGAGGCCTGCTCCTAAG |
| | | | CCTGAGGGCTGTGCCGATGCCTGTGTGA |
| | | | TCATGCCCAACGAGAACCAGAGCATCAG |
| | | | AACCGTGATCAGCGGCAGCGCCGAGAAC |
| | | | CTGGCTACACTGAAGGCTGAGTGGGAGA |
| | | | CACACAAGAGAAACGTGGACACCCTGTT |
| | | | CGCCTCTGGCAACGCTGGACTGGGCTTC |
| | | | CTGGATCCTACAGCCGCTATCGTGTCTA |
| | | | GCGACACCACAGCCTGA |
| 89 | Artificial | pVAX1_HBc- | ATGGACATCGACCCCTACAAAGAATTTG |
| | | SpyTag (DNA) | GCGCCAGCGTGGAACTGCTGAGCTTCCT |
| | | | GCCTAGCGACTTCTTCCCTTCCATCCGG |
| | | | GACCTGCTGGATACAGCCAGCGCACTGT |
| | | | ATAGAGAGGCCCTGGAATCTCCCGAGCA |
| | | | CGTGTCACCTCACCACACAGCTCTGAGA |
| | | | CAGGCCATCCTGTGTTGGGGCGAGCTGA |
| | | | TGAACCTGGCCACATGGGTCGGAAGCAA |
| | | | CCTGGAAGATCCCGCCAGCAGAGAACTG |
| | | | GTGGTGTCCTACGTGAACGTGAACATGG |
| | | | GCCTGAAGCTGAGACAGATCCTGTGGTT |
| | | | CCACATCAGCTGCCTGACCTTCGGCAGA |
| | | | GAAACCGTGCTGGAATACCTGGTGTCCT |
| | | | TCGGCGTGTGGATCAGAACCCCTACCGC |
| | | | CTACAGACCTCCTAACGCTCCCATCCTG |
| | | | AGCACCCTGCCTGAGACAACAGTTGTTG |
| | | | GCGGAGGCGGAGGATCTCCTGGCGGAGG |
| | | | AACACCTTCTCCAGGTGGTGGTGGATCT |
| | | | CAAAGTCCTGGTGGTGGCGGTTCTCAGA |
| | | | GCGGCGAATCTCAGTGTGGCTCTGCCCA |
| | | | CATCGTGATGGTGGACGCCTACAAGCCC |
| | | | ACCAAATGA |
| 90 | Artificial | pVAX1- | GAGCCATGGTGGATACACTGTCTGGACT |
| | | SPYCEGFP | GTCTAGCGAGCAGGGCCAGAGCGGCGAC |
| | | (DNA) | ATGACAATCGAGGAAGATAGCGCCACAC |
| | | | ACATCAAGTTCAGCAAGCGCGACGAGGA |
| | | | CGGCAAAGAACTGGCTGGCGCTACCATG |
| | | | GAACTGAGAGACAGCAGCGGCAAGACCA |
| | | | TCAGCACCTGGATCTCTGACGGCCAAGT |
| | | | GAAGGACTTCTATCTGTACCCCGGCAAG |
| | | | TACACCTTCGTGGAAACAGCCGCTCCTG |
| | | | ACGGATACGAGGTGGCCACAGCCATCAC |
| | | | CTTCACCGTGAACGAGCAGGGACAAGTG |
| | | | ACAGTGAACGGCAAGGCCACAAAGGGCG |
| | | | ACGCTCACATTGGCGGCTCTGGCTCCAT |
| | | | GGTGTCCAAGGGCGAAGAACTGTTCACC |
| | | | GGCGTGGTGCCCATTCTGGTGGAACTGG |
| | | | ATGGGGATGTGAACGGCCACAAGTTCTC |
| | | | CGTGTCTGGCGAAGGCGAAGGGGATGCC |
| | | | ACATACGGCAAGCTGACCCTGAAGTTCA |
| | | | TCTGCACCACCGGAAAGCTGCCCGTGCC |
| | | | TTGGCCTACACTGGTCACCACACTGACA |
| | | | TACGGCGTGCAGTGCTTCAGCAGATACC |
| | | | CCGACCATATGAAGCAGCACGACTTCTT |
| | | | CAAGAGCGCCATGCCTGAGGGCTACGTG |
| | | | CAAGAGAGAACCATCTTCTTTAAGGACG |
| | | | ACGGCAACTACAAGACCAGGGCCGAAGT |
| | | | GAAGTTCGAGGGCGACACCCTGGTCAAC |
| | | | AGAATCGAGCTGAAGGGCATCGACTTCA |
| | | | AAGAGGATGGCAACATCCTGGGCCACAA |
| | | | GCTCGAGTACAACTACAACAGCCACAAC |
| | | | GTGTACATCATGGCCGACAAGCAGAAAA |
| | | | ACGGCATCAAAGTGAACTTCAAGATCCG |
| | | | GCACAACATCGAGGACGGAAGCGTGCAG |
| | | | CTGGCCGATCACTACCAGCAGAACACAC |
| | | | CTATCGGCGACGGACCTGTGCTGCTGCC |
| | | | TGATAACCACTACCTGAGCACACAGAGC |
| | | | GCCCTGAGCAAGGACCCTAACGAGAAGA |
| | | | GGGACCACATGGTGCTGCTGGAATTTGT |
| | | | GACCGCCGCTGGCATCACACTCGGCATG |
| | | | GACGAGCTGTACAAATGA |
| 91 | Myxococcus | Encapsulin | MPDFLGHAENPLREEEWARLNETVIQVA |
| | xanthus | (EncA) | RRSLVGRRILDIYGPLGAGVQTVPYDEF |
| | | | QGVSPGAVDIVGEQETAMVFTDARKFKT |
| | | | IPIIYKDFLLHWRDIEAARTHNMPLDVS |
| | | | AAAGAAALCAQQEDELIFYGDARLGYEG |
| | | | LMTANGRLTVPLGDWTSPGGGFQAIVEA |
| | | | TRKLNEQGHFGPYAVVLSPRLYSQLHRI |
| | | | YEKTGVLEIETIRQLASDGVYQSNRLRG |
| | | | ESGVVVSTGRENMDLAVSMDMVAAYLGA |
| | | | SRMNHPFRVLEALLLRIKHPDAICTLEG |
| | | | AGATERR |
| 92 | Geobacillus sp. | 2-oxo acid | MAFEFKLPDIGEGIHEGEIVKWFVKPGD |
| | | dehydrogenase | EVNEDDVLCEVQNDKAVVEIPSPVKGKV |
| | | subunit | LEILVPEGTVATVGQMLITLDAPGYENM |
| | | | MFKGQEQEEAKKEEKTETVSKEEKVDAV |
| | | | APNAPAAEAEAGPNRRVIAMPSVRKYAR |
| | | | EKGVDIRLVQGTGKNGRVLKEDIDAFLA |
| | | | GGAKPAPAAAEEKAAPAAAKPATTEGEF |
| | | | PETREKMSGIRRAIAKAMVHSKHTAPHV |
| | | | TLMDEADVTKLVAHRKKFKAIAAEKGIK |
| | | | LTFLPYVVKALVSALREYPVLNTSIDDE |
| | | | TEEIIQKHYYNIGIAADTDRGLLVPVIK |
| | | | HADRKPIFALAQEINELAEKARDGKLTP |
| | | | GEMKGASCTITNIGSAGGQWFTPVINHP |
| | | | EVAILGIGRIAEKPIVRDGEIVAAPMLA |
| | | | LSLSFDHRMIDGATAQKALNHIKRLLSD |
| | | | PELLLMEA |
| 93 | Actinomyces | FimP domain 3 | GSLSKYGKVILTKTGTDDLADKTKYNGA |
| | viscosus | | QFQVYECTKTASGATLRDSDPSTQTVDP |
| | | | LTIGGE |
| 94 | Actinomyces | FimP domain 3 | ggcagcctgagcaaatatggcaaagtga |
| | viscosus | DNA | ttctgaccaaaaccggcaccgatgatct |
| | | | ggcggataaaaccaaatataacggcgcg |
| | | | cagtttcaggtgtatgaatgcaccaaaa |
| | | | ccgcgagcggcgcgaccctgcgcgatag |
| | | | cgatccgagcacccagaccgtggatccg |
| | | | ctgaccattggcggcgaa |
| 95 | Streptococcus | Streptococcal | GSTTKVKLIKVDQDHNRLEGVGFKLVSV |
| | pneumonia | ancillary pilin | ARDVSAAAVPLIGEYRYSSSGQVGRTLY |
| | serotype 4 | Domain 2 | TDKNGEIFVTNLPLGNYRFKEVEPLAGY |
| | (strain ATCC | | AVTTLDTDVQLVDHQLVT |
| | BAA- | | |
| | 334/TIGR4) | | |
| 96 | Streptococcus | Streptococcal | GGCAGCACCACCAAGGTGAAGCTGATCA |
| | pneumonia | ancillary pilin | AGGTGGACCAGGACCACAACAGGCTGGA |
| | serotype 4 | Domain 2 DNA | GGGCGTGGGCTTCAAGCTGGTGAGCGTG |
| | (strain ATCC | | GCCAGGGACGTGAGCGCCGCCGCCGTGC |
| | BAA- | | CCCTGATCGGCGAGTACAGGTACAGCAG |
| | 334/TIGR4) | | CAGCGGCCAGGTGGGCAGGACCCTGTAC |
| | | | ACCGACAAGAACGGCGAGATCTTCGTGA |
| | | | CCAACCTGCCCCTGGGCAACTACAGGTT |
| | | | CAAGGAGGTGGAGCCCCTGGCCGGCTAC |
| | | | GCCGTGACCACCCTGGACACCGACGTGC |
| | | | AGCTGGTGGACCACCAGCTGGTGACC |
| 97 | Streptococcus | Streptococcal | PRGNVDFMKVDGRTNTSLQGAMFKVMKE |
| | pneumonia | ancillary pilin | ESGHYTPVLQNGKEVVVTSGKDGRFRVE |
| | serotype 4 | Domain 3 | GLEYGTYYLWELQAPTGYVQLTSPVSFT |
| | (strain ATCC | | IGKDTRKELV |
| | BAA- | | |
| | 334/TIGR4) | | |
| 98 | Streptococcus | Streptococcal | CCCAGGGGCAACGTGGACTTCATGAAGG |
| | pneumonia | ancillary pilin | TGGACGGCAGGACCAACACCAGCCTGCA |
| | serotype 4 | Domain 3 DNA | GGGCGCCATGTTCAAGGTGATGAAGGAG |
| | (strain ATCC | | GAGAGCGGCCACTACACCCCCGTGCTGC |
| | BAA- | | AGAACGGCAAGGAGGTGGTGGTGACCAG |
| | 334/TIGR4) | | CGGCAAGGACGGCAGGTTCAGGGTGGAG |
| | | | GGCCTGGAGTACGGCACCTACTACCTGT |
| | | | GGGAGCTGCAGGCCCCCACCGGCTACGT |
| | | | GCAGCTGACCAGCCCCGTGAGCTTCACC |
| | | | ATCGGCAAGGACACCAGGAAGGAGCTGG |
| | | | TG |
| 99 | Corynebacterium | Major Pilin SpaD | VVTYHGKLKVVKKDGKEAGKVLKGAEFE |
| | diphtheriae | Domain 3 | LYQCTSAAVLGKGPLTVDGVKKWTTGDD |
| | NCTC 13129 | | GTFTIDGLHVTDFEDGKEAAPATKKFCL |
| | | | KETKAPAGYALPDPNVTEIEFTRAKISE |
| | | | KDKFEGDDEVT |
| 100 | Corynebacterium | Major Pilin SpaD | GTGGTGACCTACCACGGCAAGCTGAAGG |
| | diphtheriae | Domain 3 DNA | TGGTGAAGAAGGACGGCAAGGAGGCCGG |
| | NCTC 13129 | | CAAGGTGCTGAAGGGCGCCGAGTTCGAG |
| | | | CTGTACCAGTGCACCAGCGCCGCCGTGC |
| | | | TGGGCAAGGGCCCCCTGACCGTGGACGG |
| | | | CGTGAAGAAGTGGACCACCGGCGACGAC |
| | | | GGCACCTTCACCATCGACGGCCTGCACG |
| | | | TGACCGACTTCGAGGACGGCAAGGAGGC |
| | | | CGCCCCCGCCACCAAGAAGTTCTGCCTG |
| | | | AAGGAGACCAAGGCCCCCGCCGGCTACG |
| | | | CCCTGCCCGACCCCAACGTGACCGAGAT |
| | | | CGAGTTCACCAGGGCCAAGATCAGCGAG |
| | | | AAGGACAAGTTCGAGGGCGACGACGAGG |
| | | | TGACC |
| 101 | Lactobacillus | Pilin subunit | STNDTTTQNVVLTKYGFDKDVTAIDRAT |
| | rhamnosus GG | (SpaA) domain 2 | DQIWTGDGAKPLQGVDFTIYNVTANYWA |
| | | | SPKDYKGSFDSAPVAATGTTNDKGQLTQ |
| | | | ALPIQSKDASGKTRAAVYLFHETNPRAG |
| | | | YNTSADFWLTLPAKAAADGNVY |
| 102 | Lactobacillus | Pilin subunit | AGCACCAACGACACCACCACCCAGAACG |
| | rhamnosus GG | (SpaA) domain 2 | TGGTGCTGACCAAGTACGGCTTCGACAA |
| | | DNA | GGACGTGACCGCCATCGACAGGGCCACC |
| | | | GACCAGATCTGGACCGGCGACGGCGCCA |
| | | | AGCCCCTGCAGGGCGTGGACTTCACCAT |
| | | | CTACAACGTGACCGCCAACTACTGGGCC |
| | | | AGCCCCAAGGACTACAAGGGCAGCTTCG |
| | | | ACAGCGCCCCCGTGGCCGCCACCGGCAC |
| | | | CACCAACGACAAGGGCCAGCTGACCCAG |
| | | | GCCCTGCCCATCCAGAGCAAGGACGCCA |
| | | | GCGGCAAGACCAGGGCCGCCGTGTACCT |
| | | | GTTCCACGAGACCAACCCCAGGGCCGGC |
| | | | TACAACACCAGCGCCGACTTCTGGCTGA |
| | | | CCCTGCCCGCCAAGGCCGCCGCCGACGG |
| | | | CAACGTGTAC |
| 103 | Lactobacillus | Pilin subunit | TTYERTFVKKDAETKEVLEGAGFKISNS |
| | rhamnosus GG | (SpaA) domain 3 | DGKFLKLTDKDGQSVSIGEGFIDVLANN |
| | | | YRLTWVAESDATVFTSDKSGKFGLNGFA |
| | | | DNTTTYTAVETNVPDGYDAAANTDFKAD |
| | | | NS |
| 104 | Lactobacillus | Pilin subunit | ACCACCTACGAGAGGACCTTCGTGAAGA |
| | rhamnosus GG | (SpaA) domain 3 | AGGACGCCGAGACCAAGGAGGTGCTGGA |
| | | DNA | GGGCGCCGGCTTCAAGATCAGCAACAGC |
| | | | GACGGCAAGTTCCTGAAGCTGACCGACA |
| | | | AGGACGGCCAGAGCGTGAGCATCGGCGA |
| | | | GGGCTTCATCGACGTGCTGGCCAACAAC |
| | | | TACAGGCTGACCTGGGTGGCCGAGAGCG |
| | | | ACGCCACCGTGTTCACCAGCGACAAGAG |
| | | | CGGCAAGTTCGGCCTGAACGGCTTCGCC |
| | | | GACAACACCACCACCTACACCGCCGTGG |
| | | | AGACCAACGTGCCCGACGGCTACGACGC |
| | | | CGCCGCCAACACCGACTTCAAGGCCGAC |
| | | | AACAGC |
| 105 | Streptococcus | Surface protein | GQITIKKIDGSTKASLQGAIFVLKNATG |
| | agalactiae A909 | Spb1 domain 3 | QFLNFNDTNNVEWGTEANATEYTTGADG |
| | | | IITITGLKEGTYYLVEKKAPLGYNLLDN |
| | | | SQKVILGDGATDTTNSDNLLVNP |
| 106 | Streptococcus | Surface protein | GGCCAGATCACCATCAAGAAGATCGACG |
| | agalactiae A909 | Spb1 domain 3 | GCAGCACCAAGGCCAGCCTGCAGGGCGC |
| | | DNA | CATCTTCGTGCTGAAGAACGCCACCGGC |
| | | | CAGTTCCTGAACTTCAACGACACCAACA |
| | | | ACGTGGAGTGGGGCACCGAGGCCAACGC |
| | | | CACCGAGTACACCACCGGCGCCGACGGC |
| | | | ATCATCACCATCACCGGCCTGAAGGAGG |
| | | | GCACCTACTACCTGGTGGAGAAGAAGGC |
| | | | CCCCCTGGGCTACAACCTGCTGGACAAC |
| | | | AGCCAGAAGGTGATCCTGGGCGACGGCG |
| | | | CCACCGACACCACCAACAGCGACAACCT |
| | | | GCTGGTGAACCCC |
| 107 | Streptococcus | PsCsCatcher | EQDVVFSKVNVAGEEIAGAKIQLKDAQG |
| | intermedius | | QVVHSWTSKAGQSETVKLKAGTYTFHEA |
| | | | SAPTGYLAVTDITFEVDVQGKVTVKDAN |
| | | | GNGVKAD |
| 108 | Streptococcus | PsCsCatcher | GAGCAGGACGTGGTGTTCAGCAAGGTGA |
| | intermedius | (LPXTG cell wall | ACGTGGCCGGCGAGGAGATCGCCGGCGC |
| | | anchor domain- | CAAGATCCAGCTGAAGGACGCCCAGGGC |
| | | containing | CAGGTGGTGCACAGCTGGACCAGCAAGG |
| | | protein) DNA | CCGGCCAGAGCGAGACCGTGAAGCTGAA |
| | | | GGCCGGCACCTACACCTTCCACGAGGCC |
| | | | AGCGCCCCCACCGGCTACCTGGCCGTGA |
| | | | CCGACATCACCTTCGAGGTGGACGTGCA |
| | | | GGGCAAGGTGACCGTGAAGGACGCCAAC |
| | | | GGCAACGGCGTGAAGGCCGAC |
| 109 | Streptococcus | RgA Catcher | KLGDIEFIKVNKNDKKPLRGAVFSLQKQ |
| | pneumoniae | | HPDYPDIYGAIDQNGTYQNVRTGEDGKL |
| | | | TFKNLSDGKYRLFENSEPAGYKPVQNKP |
| | | | IVAFQIVNGEVRDVTSIVPQ |
| 110 | Streptococcus | RgA Catcher (cell | AAGCTGGGCGACATCGAGTTCATCAAGG |
| | pneumoniae | wall surface | TGAACAAGAACGACAAGAAGCCCCTGAG |
| | | anchor family | GGGCGCCGTGTTCAGCCTGCAGAAGCAG |
| | | protein) DNA | CACCCCGACTACCCCGACATCTACGGCG |
| | | | CCATCGACCAGAACGGCACCTACCAGAA |
| | | | CGTGAGGACCGGCGAGGACGGCAAGCTG |
| | | | ACCTTCAAGAACCTGAGCGACGGCAAGT |
| | | | ACAGGCTGTTCGAGAACAGCGAGCCCGC |
| | | | CGGCTACAAGCCCGTGCAGAACAAGCCC |
| | | | ATCGTGGCCTTCCAGATCGTGAACGGCG |
| | | | AGGTGAGGGACGTGACCAGCATCGTGCC |
| | | | CCAG |
| 111 | Corynebacterium | Major Pilin SpaD | GSERKGSLTLHKKKGAESEKRATGKEMD |
| | diphtheriae | Domain 1 | DVAGEPLNGVTFKITKLNFDLQNGDWAK |
| | NCTC 13129 | | FPKTAADAKGHETSTTKEVETSGNGTAV |
| | | | FDNLDLGIYLVEETKAPDGIVTGAPFIV |
| | | | SIPMVNEASDAWNYNVVA |
| 112 | Corynebacterium | Major Pilin SpaD | GGCAGCGAGAGGAAGGGCAGCCTGACCC |
| | diphtheriae | Domain 1 DNA | TGCACAAGAAGAAGGGCGCCGAGAGCGA |
| | NCTC 13129 | | GAAGAGGGCCACCGGCAAGGAGATGGAC |
| | | | GACGTGGCCGGCGAGCCCCTGAACGGCG |
| | | | TGACCTTCAAGATCACCAAGCTGAACTT |
| | | | CGACCTGCAGAACGGCGACTGGGCCAAG |
| | | | TTCCCCAAGACCGCCGCCGACGCCAAGG |
| | | | GCCACGAGACCAGCACCACCAAGGAGGT |
| | | | GGAGACCAGCGGCAACGGCACCGCCGTG |
| | | | TTCGACAACCTGGACCTGGGCATCTACC |
| | | | TGGTGGAGGAGACCAAGGCCCCCGACGG |
| | | | CATCGTGACCGGCGCCCCCTTCATCGTG |
| | | | AGCATCCCCATGGTGAACGAGGCCAGCG |
| | | | ACGCCTGGAACTACAACGTGGTGGCC |
| 113 | Artificial | QueenCatcher | IDTMSGLSGETGQSGNTTIEEDSTTHVK |
| | sequence | | FSKRDSNGKELAGAMIELRNLSGQTIQS |
| | | | WVSDGTVKDFYLMPGTYQFVETAAPEGY |
| | | | ELAAPITFTVNEQGQVTVNGKATKGDAH |
| | | | I |
| 114 | Artificial | QueenCatcher | ATCGACACCATGAGCGGCCTGAGCGGCG |
| | sequence | DNA | AGACCGGCCAGAGCGGCAACACCACCAT |
| | | | CGAGGAGGACAGCACCACCCACGTGAAG |
| | | | TTCAGCAAGAGGGACAGCAACGGCAAGG |
| | | | AGCTGGCCGGCGCCATGATCGAGCTGAG |
| | | | GAACCTGAGCGGCCAGACCATCCAGAGC |
| | | | TGGGTGAGCGACGGCACCGTGAAGGACT |
| | | | TCTACCTGATGCCCGGCACCTACCAGTT |
| | | | CGTGGAGACCGCCGCCCCCGAGGGCTAC |
| | | | GAGCTGGCCGCCCCCATCACCTTCACCG |
| | | | TGAACGAGCAGGGCCAGGTGACCGTGAA |
| | | | CGGCAAGGCCACCAAGGGCGACGCCCAC |
| | | | ATC |
| 115 | Artificial | MoonCake | IDTMSGLSGETGQSGNTTIEEDSTTHVK |
| | sequence | | FSKRDSNGKELAGAMIELRNLSGQTIQS |
| | | | WVSDGTVKDFYLMPGTYQFVETAAPEGY |
| | | | ELAAPITFTVQDNGEVIIQGRLTRGDVH |
| | | | I |
| 116 | Artificial | MoonCake DNA | ATCGACACCATGAGCGGCCTGAGCGGCG |
| | sequence | | AGACCGGCCAGAGCGGCAACACCACCAT |
| | | | CGAGGAGGACAGCACCACCCACGTGAAG |
| | | | TTCAGCAAGAGGGACAGCAACGGCAAGG |
| | | | AGCTGGCCGGCGCCATGATCGAGCTGAG |
| | | | GAACCTGAGCGGCCAGACCATCCAGAGC |
| | | | TGGGTGAGCGACGGCACCGTGAAGGACT |
| | | | TCTACCTGATGCCCGGCACCTACCAGTT |
| | | | CGTGGAGACCGCCGCCCCCGAGGGCTAC |
| | | | GAGCTGGCCGCCCCCATCACCTTCACCG |
| | | | TGCAGGACAACGGCGAGGTGATCATCCA |
| | | | GGGCAGGCTGACCAGGGGCGACGTGCAC |
| | | | ATC |
| 117 | Artificial | Kat I | IDTMSGLSGETGQSGNTTIEEDSTTHVK |
| | sequence | | FSKRDSNGKELAGAMIELRNLSGQTIQS |
| | | | WVSDGTVKDFYLMPGTYQFVETAAPEGY |
| | | | ELAAPITFTVQDNGEVQIQGKATRGDVP |
| | | | I |
| 118 | Artificial | Kat I DNA | ATCGACACCATGAGCGGCCTGAGCGGCG |
| | sequence | | AGACCGGCCAGAGCGGCAACACCACCAT |
| | | | CGAGGAGGACAGCACCACCCACGTGAAG |
| | | | TTCAGCAAGAGGGACAGCAACGGCAAGG |
| | | | AGCTGGCCGGCGCCATGATCGAGCTGAG |
| | | | GAACCTGAGCGGCCAGACCATCCAGAGC |
| | | | TGGGTGAGCGACGGCACCGTGAAGGACT |
| | | | TCTACCTGATGCCCGGCACCTACCAGTT |
| | | | CGTGGAGACCGCCGCCCCCGAGGGCTAC |
| | | | GAGCTGGCCGCCCCCATCACCTTCACCG |
| | | | TGCAGGACAACGGCGAGGTGCAGATCCA |
| | | | GGGCAAGGCCACCAGGGGCGACGTGCCC |
| | | | ATC |
| 119 | Artificial | Clib9 | VPTIVMVDCYKRYK |
| | sequence | | |
| 120 | Artificial | Clib9 DNA | GTGCCCACCATCGTGATGGTGGACTGCT |
| | sequence | | ACAAGAGGTACAAG |
| 121 | Artificial | TLR4 agonist | RYETMSIMIKSGGKY |
| | sequence | peptide | |
| 122 | Artificial | TLR4 agonist | AGGTACGAGACCATGAGCATCATGATCA |
| | sequence | peptide DNA | AGAGCGGCGGCAAGTAC |
| 123 | Mycobacterium | CD180/RP105 | CSSNKSTTGSGETTTA |
| | tuberculosis | agonist peptide | |
| 124 | Mycobacterium | CD180/RP105 | TGCAGCAGCAACAAGAGCACCACCGGCA |
| | tuberculosis | agonist peptide | GCGGCGAGACCACCACCGCC |
| | | DNA | |
| 125 | Mus musculus | Complement | KFLNTAKDRNRWEEPDQQLYNVEATSYA |
| | | receptor binding | |
| | | peptide (C3d) | |
| 126 | Mus musculus | Complement | AAGTTCCTGAACACCGCCAAGGACAGGA |
| | | receptor binding | ACAGGTGGGAGGAGCCCGACCAGCAGCT |
| | | peptide (C3d) | GTACAACGTGGAGGCCACCAGCTACGCC |
| | | DNA | |
| 127 | Artificial | Peptide binding to | NWYLPWLGTNDW |
| | sequence | Dendritic cell | |
| | | receptor | |
| 128 | Artificial | Peptide binding to | AACTGGTACCTGCCCTGGCTGGGCACCA |
| | sequence | Dendritic cell | ACGACTGG |
| | | receptor (DNA) | |
| 129 | Vespula lewisii | Mastoparan-7 (1) | INLKALAALAKALL |
| 130 | Vespula lewisii | Mastoparan-7 (1) | ATCAACCTGAAGGCCCTGGCCGCCCTGG |
| | | DNA | CCAAGGCCCTGCTG |
| 131 | Vespula lewisii | Mastoparan-7 (2) | INLKALAALAKKIL |
| 132 | Vespula lewisii | Mastoparan-7 (2) | ATCAACCTGAAGGCCCTGGCCGCCCTGG |
| | | DNA | CCAAGAAGATCCTG |
| 133 | Artificial | TLR2-activating | GC(S/T)GLXSIT |
| | sequence | peptide | |
| 134 | Artificial | TLR2-activating | GGCTGCAGCGGCCTGACCAGCATCACC |
| | sequence | peptide (DNA) | |
| 135 | Artificial | TH2-biasing | KWCEC |
| | sequence | Synthetic peptide | |
| | | 1 | |
| 136 | Artificial | TH2-biasing | AAGTGGTGCGAGTGC |
| | sequence | Synthetic peptide | |
| | | 1 (DNA) | |
| 137 | Artificial | TH2-biasing | KWCECKFFKFFG |
| | sequence | Synthetic peptide | |
| | | 2 | |
| 138 | Artificial | TH2-biasing | AAGTGGTGCGAGTGCAAGTTCTTCAAGT |
| | sequence | Synthetic peptide | TCTTCGGC |
| | | 2 (DNA) | |
| 139 | Artificial | TH2-biasing | KWCECEFFEFFG |
| | sequence | Synthetic peptide | |
| | | 3 | |
| 140 | Artificial | TH2-biasing | AAGTGGTGCGAGTGCGAGTTCTTCGAGT |
| | sequence | Synthetic peptide | TCTTCGGC |
| | | 3 (DNA) | |
| 141 | Pantoea sp. | Recombinant | AQVINTNSLSLLTQNNLNKSQSALGTAI |
| | | Flagellin | ERLSSGLRINSAKDDAAGQAIANRFTAN |
| | | | IKGLTQASRNANDGISIAQTTEGALNEI |
| | | | NNNLQRVRELAVQSANGTNSQSDLDSIQ |
| | | | AEITQRLNEIDRVSGQTQFNGVKVLAQD |
| | | | NTLTIQVGANDGETIDIDLKEISSKTLG |
| | | | LDGGSPLQKIDAALAQVDTLRSDLGAVQ |
| | | | NRFNSAITNLGNTVNNLSSARSRIEDSD |
| | | | YATEVSNMSRAQILQQAGTSVLAQANQV |
| | | | PQNVLSLLR |
| 142 | Pantoea sp. | Recombinant | GCACAGGTGATTAATACCAATAGCCTGA |
| | | Flagellin DNA | GCCTGCTGACCCAGAATAATCTGAATAA |
| | | | AAGCCAGAGCGCACTGGGCACCGCAATT |
| | | | GAACGTCTGAGCAGCGGTCTGCGTATTA |
| | | | ATAGCGCCAAAGATGATGCAGCCGGTCA |
| | | | GGCAATTGCAAATCGTTTTACCGCAAAC |
| | | | ATTAAAGGTCTGACCCAGGCAAGCCGTA |
| | | | ATGCAAATGATGGTATTAGCATTGCACA |
| | | | GACCACCGAAGGTGCACTGAATGAAATT |
| | | | AACAATAATCTGCAGCGTGTTCGTGAAC |
| | | | TGGCCGTTCAGAGCGCAAATGGTACAAA |
| | | | TAGCCAGAGTGATCTGGATAGCATTCAG |
| | | | GCAGAAATTACCCAGCGTCTGAACGAAA |
| | | | TTGATCGTGTTAGCGGTCAGACCCAGTT |
| | | | TAATGGTGTTAAAGTTCTGGCACAGGAT |
| | | | AACACCCTGACCATTCAGGTTGGTGCCA |
| | | | ATGATGGCGAAACCATTGATATTGATCT |
| | | | GAAAGAGATTAGCAGCAAAACCCTGGGT |
| | | | TTAGATGGTGGTAGTCCGCTGCAGAAAA |
| | | | TTGATGCAGCACTGGCCCAGGTTGATAC |
| | | | CCTGCGTAGCGATCTGGGTGCAGTTCAG |
| | | | AATCGCTTTAATAGTGCAATTACCAATC |
| | | | TGGGCAATACCGTTAATAATCTGTCAAG |
| | | | CGCACGTAGCCGTATTGAAGATAGCGAT |
| | | | TATGCAACCGAAGTTAGCAATATGAGCC |
| | | | GTGCACAGATTCTGCAGCAGGCAGGCAC |
| | | | CAGCGTTCTGGCGCAGGCAAATCAGGTT |
| | | | CCGCAGAATGTTCTGAGTCTGCTGCGT |
| 143 | Acinetobacter | AP205 | MANKPMQPITSTANKIVWSDPTRLSTTF |
| | phage AP205 | | SASLLRQRVKVGIAELNNVSGQYVSVYK |
| | | | RPAPKPEGCADACVIMPNENQSIRTVIS |
| | | | GSAENLATLKAEWETHKRNVDTLFASGN |
| | | | AGLGFLDPTAAIVSSDTTA |
| 144 | Acinetobacter | AP205 DNA | ATGGCGAACAAACCGATGCAGCCGATTA |
| | phage AP205 | | CCAGCACCGCGAACAAAATTGTGTGGAG |
| | | | CGATCCGACCCGCCTGAGCACCACCTTT |
| | | | AGCGCGAGCCTGCTGCGCCAGCGCGTGA |
| | | | AAGTGGGCATTGCGGAACTGAACAACGT |
| | | | GAGCGGCCAGTATGTGAGCGTGTATAAA |
| | | | CGCCCGGCGCCGAAACCGGAAGGCTGCG |
| | | | CGGATGCGTGCGTGATTATGCCGAACGA |
| | | | AAACCAGAGCATTCGCACCGTGATTAGC |
| | | | GGCAGCGCGGAAAACCTGGCGACCCTGA |
| | | | AAGCGGAATGGGAAACCCATAAACGCAA |
| | | | CGTGGATACCCTGTTTGCGAGCGGCAAC |
| | | | GCGGGCCTGGGCTTTCTGGATCCGACCG |
| | | | CGGCGATTGTGAGCAGCGATACCACCGC |
| | | | G |
| 145 | SARS-COV-2 | Spike protein | RVQPTESIVRFPNITNLCPFGEVFNATR |
| | | RBD | FASVYAWNRKRISNCVADYSVLYNSASF |
| | | | STFKCYGVSPTKLNDLCFTNVYADSFVI |
| | | | RGDEVRQIAPGQTGKIADYNYKLPDDFT |
| | | | GCVIAWNSNNLDSKVGGNYNYLYRLFRK |
| | | | SNLKPFERDISTEIYQAGSTPCNGVEGF |
| | | | NCYFPLQSYGFQPTNGVGYQPYRVVVLS |
| | | | FELLHAPATVCGPKKSTNLVKNKCVNEN |
| | | | FNGLTGTGVLTESNKKELPFQQFGRDIA |
| | | | DTTDAVRDPQTLEILDITPCS |
| 146 | SARS-COV-2 | Spike protein | CGCGTGCAGCCCACCGAGAGCATCGTGC |
| | | RBD (DNA) | GCTTCCCCAACATCACCAACCTGTGCCC |
| | | | CTTCGGCGAGGTGTTCAACGCCACCCGC |
| | | | TTCGCCAGCGTGTACGCCTGGAACCGCA |
| | | | AGCGCATCAGCAACTGCGTGGCCGATTA |
| | | | CAGCGTGCTGTACAACAGCGCCAGCTTC |
| | | | AGCACCTTCAAGTGCTACGGCGTGAGCC |
| | | | CCACCAAGCTGAACGATCTGTGCTTCAC |
| | | | CAACGTGTACGCCGATAGCTTCGTGATC |
| | | | CGCGGCGATGAGGTGCGCCAGATCGCCC |
| | | | CCGGCCAGACCGGCAAGATCGCCGATTA |
| | | | CAACTACAAGCTGCCCGATGATTTCACC |
| | | | GGCTGCGTGATCGCCTGGAACAGCAACA |
| | | | ACCTGGATAGCAAGGTGGGCGGCAACTA |
| | | | CAACTACCTGTACCGCCTGTTCCGCAAG |
| | | | AGCAACCTGAAGCCCTTCGAGCGCGATA |
| | | | TCAGCACCGAGATCTACCAGGCCGGCAG |
| | | | CACCCCCTGCAACGGCGTGGAGGGCTTC |
| | | | AACTGCTACTTCCCCCTGCAGAGCTACG |
| | | | GCTTCCAGCCCACCAACGGCGTGGGCTA |
| | | | CCAGCCCTACCGCGTGGTGGTGCTGAGC |
| | | | TTCGAGCTGCTGCACGCCCCCGCCACCG |
| | | | TGTGCGGCCCCAAGAAGAGCACCAACCT |
| | | | GGTGAAGAACAAGTGCGTGAACTTCAAC |
| | | | TTCAACGGCCTGACCGGCACCGGCGTGC |
| | | | TGACCGAGAGCAACAAGAAGTTCCTGCC |
| | | | CTTCCAGCAGTTCGGCCGCGATATCGCC |
| | | | GATACCACCGATGCCGTGCGCGATCCCC |
| | | | AGACCCTGGAGATCCTGGATATCACCCC |
| | | | CTGCAGC |
| 147 | Streptococcus | SpyLigase | DYDGQSGDGKELAGATMELRDSSGKTIS |
| | pyogenes | | TWISDGQVKDFYLYPGKYTFVETAAPDG |
| | | | YEVATAITFTVNEQGQVTVNGKATKGGS |
| | | | GGSGGSGEDSATHI |
| 148 | Streptococcus | SpyLigase DNA | gattacgacggtcagtccggtgacggca |
| | pyogenes | | aagagttagctggtgcaactatggagtt |
| | | | gcgtgattcatctggtaaaactattagt |
| | | | acatggatttcagatggacaagtgaaag |
| | | | atttctacctgtatccaggaaaatatac |
| | | | atttgtcgaaaccgcagcaccagacggt |
| | | | tatgaggtagcaactgctattaccttta |
| | | | cagttaatgagcaaggtcaggttactgt |
| | | | aaatggcaaagcaactaaaggtgggagt |
| | | | ggtggcagcggaggtagtggcgaggaca |
| | | | gcgctacccatatt |
| 149 | Artificial | Scfv DEC-205 | EVKLQQSGTEVVKPGASVKLSCKASGYI |
| | sequence | | FTSYDIDWVRQTPEQGLEWIGWIFPGEG |
| | | | STEYNEKFKGRATLSVDKSSSTAYMELT |
| | | | RLTSEDSAVYFCARGDYYRRYFDLWGQG |
| | | | TTVTVSSGGGGSGGGGSGGGGSDIQMTQ |
| | | | SPSFLSTSLGNSITITCHASQNIKGWLA |
| | | | WYQQKSGNAPQLLIYKASSLQSGVPSRF |
| | | | SGSGSGTDYIFTISNLQPEDIATYYCQH |
| | | | YQSFPWTFGGGTKLEIKRAA |
| 150 | Artificial | Scfv DEC-205 | GAAGTGAAGCTGCAGCAGAGCGGAACCG |
| | sequence | DNA | AGGTTGTGAAGCCAGGTGCCAGTGTGAA |
| | | | GCTGAGCTGCAAGGCCAGCGGCTACATC |
| | | | TTCACCAGCTACGACATCGATTGGGTCC |
| | | | GACAGACCCCAGAGCAAGGCCTGGAATG |
| | | | GATCGGCTGGATTTTCCCAGGCGAGGGC |
| | | | AGCACCGAGTACAACGAGAAGTTTAAGG |
| | | | GACGCGCCACGCTGTCCGTGGATAAGAG |
| | | | TAGTAGCACCGCCTACATGGAACTGACC |
| | | | CGCCTGACAAGCGAAGATAGCGCCGTGT |
| | | | ACTTTTGCGCCCGTGGCGATTACTACCG |
| | | | CCGCTACTTTGATCTGTGGGGCCAGGGA |
| | | | ACCACCGTGACAGTTAGTTCAGGTGGCG |
| | | | GAGGTAGCGGAGGTGGTGGATCAGGTGG |
| | | | TGGCGGAAGCGATATTCAGATGACCCAG |
| | | | TCGCCGAGCTTCCTGAGCACAAGCCTGG |
| | | | GCAACAGCATCACCATTACCTGCCACGC |
| | | | CAGCCAGAACATCAAAGGCTGGCTGGCC |
| | | | TGGTATCAGCAGAAGTCCGGAAACGCCC |
| | | | CACAGCTGCTGATCTACAAGGCTAGCAG |
| | | | CCTGCAAAGCGGAGTGCCAAGCCGTTTT |
| | | | AGCGGAAGTGGAAGCGGCACCGATTACA |
| | | | TTTTCACCATCAGCAATCTGCAGCCCGA |
| | | | GGACATTGCCACCTACTACTGCCAGCAC |
| | | | TACCAGAGCTTCCCGTGGACATTCGGCG |
| | | | GAGGCACCAAGCTGGAAATCAAGCGTGC |
| | | | TGCC |
| 151 | Thermotoga | Encapsulin | MEFLKRSFAPLTEKQWQEIDNRAREIFK |
| | maritima | | TQLYGRKFVDVEGPYGWEYAAHPLGEVE |
| | | | VLSDENEVVKWGLRKSLPLIELRATFTL |
| | | | DLWELDNLERGKPNVDLSSLEETVRKVA |
| | | | EFEDEVIFRGCEKSGVKGLLSFEERKIE |
| | | | CGSTPKDLLEAIVRALSIFSKDGIEGPY |
| | | | TLVINTDRWINFLKEEAGHYPLEKRVEE |
| | | | CLRGGKIITTPRIEDALVVSERGGDFKL |
| | | | ILGQDLSIGYEDREKDAVRLFITETFTF |
| | | | QVVNPEALILLKF |
| 152 | Pyrococcus | 2E0Z | MLSINPTLINRDKPYTKEELMEILRLAI |
| | furiosus | | IAELDAINLYEQMARYSEDENVRKILLD |
| | | | VAREEKAHVGEFMALLLNLDPEQVTELK |
| | | | GGFEEVKELTGIEAHINDNKKEESNVEY |
| | | | FEKLRSALLDGVNKGRSLLKHLPVTRIE |
| | | | GQSFRVDIIKFEDGVRVVKQEYKPIPLL |
| | | | KKKFYVGIRELNDGTYDVSIATKAGELL |
| | | | VKDEESLVIREILSTEGIKKMKLSSWDN |
| | | | PEEALNDLMNALQEASNASAGPFGLIIN |
| | | | PKRYAKLLKIYEKSGKMLVEVLKEIFRG |
| | | | GIIVTLNIDENKVIIFANTPAVLDVVVG |
| | | | QDVTLQELGPEGDDVAFLVSEAIGIRIK |
| | | | NPEAIVVLE |
| 153 | E. coli | Biotin acceptor | GLNDIFEAQKIEWHE |
| | | peptide | |
| 154 | E. coli | Biotin acceptor | GGCCTGAACGACATCTTCGAGGCCCAGA |
| | | peptide (DNA) | AGATCGAGTGGCACGAG |
| 155 | Streptomyces | Monomeric | AEAGITGTWYNQHGSTFTVTAGADGNLT |
| | avidinii | Streptavidin | GQYENRAQGTGCQNSPYTLTGRYNGTKL |
| | | (mSA) 2 | EWRVEWNNSTENCHSRTEWRGQYQGGAE |
| | | | ARINTQWNLTYEGGSGPATEQGQDTFTK |
| | | | VKPSAASGS |
| 156 | Streptomyces | Monomeric | GCCGAGGCCGGCATCACCGGCACCTGGT |
| | avidinii | Streptavidin | ACAACCAGCACGGCAGCACCTTCACCGT |
| | | (mSA) 2 (DNA) | GACCGCCGGCGCCGACGGCAACCTGACC |
| | | | GGCCAGTACGAGAACAGGGCCCAGGGCA |
| | | | CCGGCTGCCAGAACAGCCCCTACACCCT |
| | | | GACCGGCAGGTACAACGGCACCAAGCTG |
| | | | GAGTGGAGGGTGGAGTGGAACAACAGCA |
| | | | CCGAGAACTGCCACAGCAGGACCGAGTG |
| | | | GAGGGGCCAGTACCAGGGCGGCGCCGAG |
| | | | GCCAGGATCAACACCCAGTGGAACCTGA |
| | | | CCTACGAGGGCGGCAGCGGCCCCGCCAC |
| | | | CGAGCAGGGCCAGGACACCTTCACCAAG |
| | | | GTGAAGCCCAGCGCCGCCAGCGGCAGC |
| 157 | Artificial | I53-50A | EKAAKAEEAARKMEELFKKHKIVAVLRA |
| | sequence | | NSVEEAIEKAVAVFAGGVHLIEITFTVP |
| | | | DADTVIKALSVLKEKGAIIGAGTVTSVE |
| | | | QARKAVESGAEFIVSPHLDEEISQFAKE |
| | | | KGVFYMPGVMTPTELVKAMKLGHTILKL |
| | | | FPGEVVGPQFVKAMKGPFPNVKFVPTGG |
| | | | VNLDNVAEWFKAGVLAVGVGSALVKGTP |
| | | | DEVREKAKAFVEKIRGATE |
| 158 | Artificial | I53-50A (DNA) | GAAAAAGCGGCGAAAGCGGAAGAAGCGG |
| | sequence | | CGCGCAAAATGGAAGAACTGTTTAAAAA |
| | | | ACATAAAATTGTGGCGGTGCTGCGCGCG |
| | | | AACAGCGTGGAAGAAGCGATTGAAAAAG |
| | | | CGGTGGCGGTGTTTGCGGGCGGCGTGCA |
| | | | TCTGATTGAAATTACCTTTACCGTGCCG |
| | | | GATGCGGATACCGTGATTAAAGCGCTGA |
| | | | GCGTGCTGAAAGAAAAAGGCGCGATTAT |
| | | | TGGCGCGGGCACCGTGACCAGCGTGGAA |
| | | | CAGGCGCGCAAAGCGGTGGAAAGCGGCG |
| | | | CGGAATTTATTGTGAGCCCGCATCTGGA |
| | | | TGAAGAAATTAGCCAGTTTGCGAAAGAA |
| | | | AAAGGCGTGTTTTATATGCCGGGCGTGA |
| | | | TGACCCCGACCGAACTGGTGAAAGCGAT |
| | | | GAAACTGGGCCATACCATTCTGAAACTG |
| | | | TTTCCGGGCGAAGTGGTGGGCCCGCAGT |
| | | | TTGTGAAAGCGATGAAAGGCCCGTTTCC |
| | | | GAACGTGAAATTTGTGCCGACCGGCGGC |
| | | | GTGAACCTGGATAACGTGGCGGAATGGT |
| | | | TTAAAGCGGGCGTGCTGGCGGTGGGCGT |
| | | | GGGCAGCGCGCTGGTGAAAGGCACCCCG |
| | | | GATGAAGTGCGCGAAAAAGCGAAAGCGT |
| | | | TTGTGGAAAAAATTCGCGGCGCGACCGA |
| | | | A |
| 159 | Artificial | I53-50B | MDVNMKLTGRIMDAAKEVDHTCRSSTGV |
| | sequence | | PRDMLHRYAEGQTVDDDDFKCYLKCIMV |
| | | | EFNSLSDDGVFVLEEELENVPPEIKEEG |
| | | | HRVVHSCKHINHDEACETAYQIHQCYKQ |
| | | | SDPELYSLVVRAFDATIGD |
| 160 | Artificial | I53-50B (DNA) | ATGGATGTGAACATGAAACTGACCGGCC |
| | sequence | | GCATTATGGATGCGGCGAAAGAAGTGGA |
| | | | TCATACCTGCCGCAGCAGCACCGGCGTG |
| | | | CCGCGCGATATGCTGCATCGCTATGCGG |
| | | | AAGGCCAGACCGTGGATGATGATGATTT |
| | | | TAAATGCTATCTGAAATGCATTATGGTG |
| | | | GAATTTAACAGCCTGAGCGATGATGGCG |
| | | | TGTTTGTGCTGGAAGAAGAACTGGAAAA |
| | | | CGTGCCGCCGGAAATTAAAGAAGAAGGC |
| | | | CATCGCGTGGTGCATAGCTGCAAACATA |
| | | | TTAACCATGATGAAGCGTGCGAAACCGC |
| | | | GTATCAGATTCATCAGTGCTATAAACAG |
| | | | AGCGATCCGGAACTGTATAGCCTGGTGG |
| | | | TGCGCGCGTTTGATGCGACCATTGGCGA |
| | | | T |
| 161 | Streptococcus | PsCsTag | GNKLTVTDQAAPS |
| | intermedius | | |
| 162 | Streptococcus | PsCsTag (DNA) | GGCAACAAGCTGACCGTGACCGACCAGG |
| | intermedius | | CCGCCCCCAGC |
| 163 | Streptococcus | KTag | ATHIKFSKRD |
| | pyogenes | | |
| 164 | Streptococcus | KTag (DNA) | GCCACCCACATCAAGTTCAGCAAGAGGG |
| | pyogenes | | AC |
| 165 | Streptococcus | DogTag | DIPATYEFTDGKHYITNEPIPPK |
| | pneumoniae | | |
| 166 | Streptococcus | DogCatcher | GAMKLGEIEFIKVDKTDKKPLRGAVFSL |
| | pneumoniae | | QKQHPDYPDIYGAIDQNGTYQDVRTGED |
| | | | GKLIFTNLSDGKYRLIENSEPPGYKPVQ |
| | | | NKPIVSFRIVDGEVRDVTSIVPQ |
| 167 | Artificial | CL3 | GWKERLSSWNRF |
| | sequence | | |
| 168 | Artificial | Co1 | SFHQLPARSPLP |
| | sequence | | |
| 169 | Artificial | CKS9 | CKSTHPLSC |
| | sequence | | |
| 170 | Artificial | Peptide 3 | FYPSYHSTPQRP |
| | sequence | | |
| 171 | Artificial | Peptide 12 | AYYKTASLAPAE |
| | sequence | | |
| 172 | Artificial | Peptide 18 | SLSLLTMPGNAS |
| | sequence | | |
| 173 | Artificial | IRMM_01 | YTYQGKL |
| | sequence | | |
| 174 | Artificial | IRMM_02 | CYTYQGKLC |
| | sequence | | |
| 175 | Homo Sapiens | p17 | LNCSNCPQPQNSSLRTPLRQGGSAGSAG |
| | | | SAG |
| 176 | Homo Sapiens | p18 | SLERFTGLDLANVTLTYEFAAGPRDFWQ |
| | | | PGSAGSAGSAG |
| 177 | Artificial | p30 | GSGSGSGSCGRWSGWPADLC |
| | sequence | | |
| 178 | Artificial | PCP1 | HSIFTAFPGSRP |
| | sequence | | |
| 179 | Artificial | PCP3 | ATYSEFPGNLKP |
| | sequence | | |
| 180 | Artificial | PCP4 | SMPMAYIPFPGN |
| | sequence | | |
| 181 | Artificial | XS52.1 | GPEDTSRAPENQQKTFHRRW |
| | sequence | | |
| 182 | Artificial | IRMM_03 | FGPNLTGRW |
| | sequence | | |
| 183 | Artificial | IRMM_04 | DLXTSK(M/L)X(V/I/L) |
| | sequence | | |
| 184 | Artificial | IRMM_05 | HFYLPM |
| | sequence | | |
| 185 | Artificial | IRMM_06 | MFYLPM |
| | sequence | | |
| 186 | Artificial | IRMM_07 | (K/R/M)KYY(P/V/Y)M |
| | sequence | | |
| 187 | Artificial | C-tag purification | EPEA |
| | sequence | tag | |
| 188 | Artificial | Linker_01 | GSGS |
| | sequence | | |
| 189 | Artificial | Linker_02 | GSGTAGGGSGS |
| | sequence | | |

### REFERENCES

De Vincenzo, R.; Conte, C.; Ricci, C.; Scambia, G.; Capelli, G. Long-term efficacy and safety of human papillomavirus vaccination. Int. J. Womens. Health 2014, 6, 999-1010.
DiLillo D J, Tan G S, Palese P, Ravetch J V. Broadly neutralizing hemagglutinin stalk-specific antibodies require FcγR interactions for protection against influenza virus in vivo. Nat Med. 2014; 20(2):143-151. doi:10.1038/nm.3443
Fischinger S, Dolatshahi S, Jennewein M F, et al. IgG3 collaborates with IgG1 and IgA to recruit effector function in RV144 vaccinees. JCI Insight. 2020; 5(21):e140925. Published 2020 Nov 5. doi:10.1172/jci.insight.140925
Ilva Lieknina, Gints Kalninš, Ināra Akopjana, Jānis Bogans, Mihails Šišovs, Juris Jansons, Jānis Rūmnieks, Kaspars Tārs: Production and characterization of novel ssRNA bacteriophage virus-like particles from metagenomic sequencing data; J Nanobiotechnology. 2019; 17: 61.
Keeble A H, Yadav V K, Ferla M P, et al. DogCatcher allows loop-friendly protein-protein ligation [published online ahead of print, 2021 Jul 28]. Cell Chem Biol. 2021; S2451-9456(21)00315-9. doi:10.1016/j.chembiol.2021.07.005
Kumar S, Sunagar R, Gosselin E. Bacterial Protein Toll-Like-Receptor Agonists: A Novel Perspective on Vaccine Adjuvants. Front Immunol. 2019 May 29; 10:1144. doi: 10.3389/fimmu.2019.01144. PMID: 31191528; PMCID: PMC6549121.
Lieknina I, Černova D, Rǔmnieks J, Tārs K. Novel ssRNA phage VLP platform for displaying foreign epitopes by genetic fusion. Vaccine. 2020 Aug. 27; 38(38):6019-6026. doi: 10.1016/j.vaccine.2020.07.016. Epub 2020 Jul 24. PMID: 32713683.
Mendoza J L, Escalante N K, Jude K M, et al. Structure of the IFNγ receptor complex guides design of biased agonists. Nature. 2019; 567(7746):56-60. doi:10.1038/s41586-019-0988-7
Patterson D, Schwarz B, Avera J, et al. Sortase-Mediated Ligation as a Modular Approach for the Covalent Attachment of Proteins to the Exterior of the Bacteriophage P22 Virus-like Particle. Bioconjug Chem. 2017; 28(8):2114-2124. doi:10.1021/acs.bioconjchem.7b00296
Schiller, J.; Lowy, D. Explanations for the high potency of HPV prophylactic vaccines. Vaccine 2018, 36, 4768-4773.
Schiller, J. T.; Castellsagué, X.; Garland, S. M. A review of clinical trials of human papillomavirus prophylactic vaccines. Vaccine 2012, 30, F123-F138.
Tang S, Xuan B, Ye X, Huang Z, Qian Z. A Modular Vaccine Development Platform Based on Sortase-Mediated Site-Specific Tagging of Antigens onto Virus-Like Particles. Sci Rep. 2016; 6:25741. Published 2016 May 12. doi:10.1038/srep25741
Von Holle T A, Moody M A. Influenza and Antibody-Dependent Cellular Cytotoxicity. Front Immunol. 2019; 10:1457. Published 2019 Jun 25. doi:10.3389/fimmu.2019.01457
Wadhwa M, Knezevic I, Kang H N, Thorpe R. Immunogenicity assessment of biotherapeutic products: An overview of assays and their utility. Biologicals. 2015; 43(5):298-306. doi:10.1016/j.biologicals.2015.06.004
Zakeri, B. et al. J. Am. Chem. Soc., 2010, 132 (13), pp 4526-4527
Zakeri, B. et al. Proceedings of the National Academy of Sciences 109(12), E690-E697. 2012.

### Items

1. A method of modulating an immune response of a subject to an antigen, the method comprising a step of administering a composition to said subject at least once, wherein the composition comprises:
   i) a particle-forming protein fused to a first peptide tag and, optionally, to a third peptide tag, optionally by a linker;
   ii) said antigen fused to a second peptide tag, optionally by a linker; and
   iii) at least one immune response modulating moiety (IRMM),
wherein
a) i), ii) and iii) are provided as one or more polypeptides or as one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond or by affinity conjugation, optionally by a linker; or
   ii. is fused to a fourth peptide tag, optionally by a linker; and
c) the antigen and the particle-forming protein are capable of being linked via an isopeptide bond, by an ester bond, by chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
whereby i)-ii)-iii) form a particle displaying said antigen and said at least one IRMM.
2. A method of modulating an immune response of a subject to an antigen, the method comprising a step of administering a composition to said subject at least once, wherein the composition comprises:
   i) a particle-forming protein fused to said antigen, optionally by a linker, and, optionally, to a third peptide tag, optionally by a linker; and
   ii) at least one immune response modulating moiety (IRMM)
wherein
a) i) and ii) are provided as one or more polypeptides or as one or more polynucleotides encoding i) and ii);
b) said at least one IRMM
   i. is linked to said particle-forming protein or said antigen by fusion, by an isopeptide bond, by an ester bond or by affinity conjugation, optionally by a linker; or
   ii. is fused to a fourth peptide tag, optionally by a linker; and
c) the at least one IRMM and the particle-forming protein are capable of being linked via an isopeptide bond, via an ester bond, via chemical crosslinking or via affinity conjugation between the third and the fourth peptide tags when present,
whereby i)-ii) form a particle displaying said antigen and said at least one IRMM.
3. The method according to any one the preceding items, wherein i), ii) and iii) are provided as one or more polynucleotides encoding i), ii) and iii);
wherein said particle-forming protein fused to said first peptide tag, and optionally to said third peptide tag, is encoded by a first polynucleotide.
4. The method according to any one of items 1 or 3, wherein i), ii) and iii) are provided as one or more polynucleotides encoding i), ii) and iii);
wherein said antigen fused to said second peptide tag is encoded by a second polynucleotide.
5. The method according to any one of the preceding items, wherein i), ii) and iii) are provided as one or more polynucleotides encoding i), ii) and iii);
wherein said at least one IRMM fused to a fourth peptide tag is encoded by a third polynucleotide.
6. The method according to any one of items 3 to 4, wherein i), ii) and iii) are provided as one or more polynucleotides encoding i), ii) and iii);
wherein said first and/or second polynucleotide further encodes said at least one IRMM.
7. The method according to any one of the preceding items, wherein the particle is a virus-like particle.
8. The method according to any one of the preceding items, wherein the particle is a nanoparticle.
9. The method according to any one of the preceding items, wherein the particle displays the antigen on an internal or on an external surface, preferably wherein the particle displays the antigen on an external surface.
10. The method according to any one of items 3 to 8, wherein the first, second and/or third polynucleotide is DNA.
11. The method according to any one of items 3 to 10, wherein the first, second and/or third polynucleotide is RNA.
12. The method according to item 11, wherein the RNA is formulated in a lipid particle formulation.
13. The method according to any one of the preceding items, wherein the particle-forming protein is a viral capsid protein or a viral envelope protein, such as a glycoprotein.
14. The method according to any one of the preceding items, wherein the particle-forming protein is a viral capsid protein or a viral envelope protein from a mammalian virus, such as a human virus.
15. The method according to item 14, wherein the particle-forming protein is a particle-forming protein from a hepatitis virus such as hepatitis B or E, for example a core protein from hepatitis B virus; a particle-forming protein from a norovirus such as NoV; a particle-forming protein from a papilloma virus such as Human Papilloma Virus (HPV), preferably HPV16 or HPV18, such as HPV L1; a particle-forming protein from a polyomavirus such as polyomavirus vp1 (PyV); a particle-forming protein from a calicivirus such as feline calicivirus (FCV), preferably FCV VP1; a particle-forming protein from a circovirus such as a porcine circovirus (PCV), preferably PCV2 ORF2; a particle-forming protein from a nervous necrosis virus (NNV), such as NNV coat protein; a particle-forming protein from a parvovirus such as canine parvovirus (CVP), preferably CPV VP2, goose parvovirus (GPV) or porcine parvovirus (PPV), preferably structural proteins from GPV or PPV, or parvovirus B19; a particle-forming protein from a protoparvovirus such as an enteritis virus, for example mink enteritis virus (MEV), preferably MEV VP2, or duck plague virus (DPV), preferably a DPV structural protein, or a viral Gag protein, such as a HIV Gag.
16. The method according to any one of the preceding items, wherein the particle-forming protein is a particle-forming protein from a plant virus, such as a cowpea virus, a tobacco virus, a tomato virus, a cucumber virus or a potato virus.
17. The method according to item 16, wherein the plant virus is a mosaic virus, preferably Cowpea mosaic virus (CPMV); tobacco mosaic virus (TMV); tomato spotted wilt virus (TSWV); tomato yellow leaf curl virus (TYLCV); cucumber mosaic virus (CMV); or from potato virus Y (PVY).
18. The method according to any one of the preceding items, wherein the particle-forming protein is a bacteriophage protein, such as a particle-forming protein from Salmonella virus P22, from MS2, from QBeta, PRR1, PP7, bacteriophage R17, bacteriophage fr, bacteriophage GA, bacteriophage SP, bacteriophage M11, bacteriophage MX1, bacteriophage NL95, bacteriophage f2 or Cb5, or such as wherein the particle-forming protein is GA-879, NF-391, ESE002, or AP205.
19. The method according to any one of the preceding items, wherein the particle-forming protein is a ferritin (SEQ ID NO: 69), an i301 (SEQ ID NO: 72), a replicase polyprotein 1a (pp1a), a lumazine synthase (SEQ ID NO: 70), an encapsulin (SEQ ID NO: 91), 2-oxo acid dehydrogenase subunit E2 (SEQ ID NO: 92), an encapsulin (SEQ ID NO: 151), a 2E0Z (SEQ ID NO: 152), or respective homologues thereof having at least 60% sequence identity thereto.
20. The method according to any one of the preceding items, wherein the IRMM is fused at the N-terminus of the particle-forming protein, at the C-terminus of the particle-forming protein, or within an internal region, such as in an exposed loop, of the particle-forming protein, optionally by a linker.
21. The method according to any one of the preceding items, wherein a first and a second IRMMs are fused, optionally by one or more linkers, to one or two fusion locations selected from the group consisting of the N-terminus of the particle-forming protein, the C-terminus of the particle-forming protein, within an internal region, such as in an exposed loop, of the particle-forming protein, the first peptide tag, the second peptide tag and the antigen.
22. The method according to any one of the preceding items, wherein a first, a second and a third IRMMs are fused, optionally by one or more linkers, to one, two or three fusion locations selected from the group consisting of the N-terminus of the particle-forming protein, the C-terminus of the particle-forming protein, an internal region, such as an exposed loop, of the particle-forming protein, the first peptide tag, the second peptide tag and the antigen.
23. The method according to any one of the proceeding items, wherein the IRMM is fused to a further IRMM, optionally by a linker.
24. The method according to to any one of the proceeding items, wherein the particle-forming protein comprises an internal IRMM.
25. The method according to any one of the preceding items, wherein the IRMM and/or the further IRMM is an immune cell targeting moiety, such as wherein the IRMM and/or the further IRMM is capable of binding to a surface receptor of an immune cell, optionally wherein the further IRMM is the first, second or third IRMM.
26. The method according to any one of the preceding items, wherein the IRMM and/or the further IRMM is selected from the group consisting of a Toll-like receptor (TLR) agonist, a dendritic cell receptor binding moiety, a mast cell receptor binding moiety, a universal T cell epitope, a Complement component 3 (C3) binding moiety, an epitope recognized by a regulatory T cell, a Dectin-1 receptor targeting moiety, a cytokine or a chemokine, optionally wherein the further IRMM is the first, second or third IRMM.
27. The method according to item 26, wherein the TLR agonist is an agonist of TLR4, such as a peptide according to SEQ ID NO: 121 or a homologue thereof having at least 60% sequence identity thereto; an agonist of CD180, such as a peptide according to SEQ ID NO: 123 or a homologue thereof having at least 60% sequence identity thereto; an agonist of TLR5, such as a recombinant flagellin according to SEQ ID NO: 141 or a homologue thereof having at least 60% sequence identity thereto; or an agonist of TLR2, such as a peptide according to SEQ ID NO: 133 or a homologue thereof having at least 60% sequence identity thereto.
28. The method according to item 26, wherein the dendritic cell receptor binding moiety is a peptide according to SEQ ID NO: 127 or a homologue thereof having at least 60% sequence identity thereto, or DEC-205 (SEQ ID NO: 149) or a homologue thereof having at least 60% sequence identity thereto.
29. The method according to item 26, wherein the mast cell receptor binding moiety is a Mastoparan-7, such as a peptide according to SEQ ID NO: 129, SEQ ID NO: 131, or respective homologues thereof having at least 60% sequence identity thereto.
30. The method according to item 26, wherein the C3 binding moiety is a peptide according to SEQ ID NO: 125 or a homologue thereof having at least 60% sequence identity thereto.
31. The method according to any one the preceding items, wherein the IRMM is a T helper cell type 1 (Th1) response-biasing peptide.
32. The method according to any one the preceding items, wherein the IRMM is a T helper cell type 2 (Th2) response-biasing peptide.
33. The method according to item 32, wherein the Th2 response-biasing peptide is a synthetic Th2 response-biasing peptide, such as a peptide according to SEQ ID NO: 135, SEQ ID NO: 137, SEQ ID NO: 139, or respective homologues thereof having at least 60% sequence identity thereto.
34. The method according to any one of items 1 and 3 to 26, wherein the particle-forming protein is AP205 (SEQ ID NO: 143) or a homologue thereof having at least 60% sequence identity thereto, the first peptide tag is a RumTrunk tag (SEQ ID NO: 14) or a homologue thereof having at least 60% sequence identity thereto, the second peptide tag is a MoonCake (SEQ ID NO: 115) or a homologue thereof having at least 60% sequence identity thereto, and the IRMM is the CD180 agonist peptide according to SEQ ID NO: 123 or a homologue thereof having at least 60% sequence identity thereto, wherein
   i) the second peptide tag is fused to the antigen, optionally by a linker; and
   ii) the first peptide tag and the IRMM are fused to the particle-forming protein, optionally by separate linkers.
35. The method according to any one of items 1 and 3 to 26, wherein the particle-forming protein is AP205 (SEQ ID NO: 143) or a homologue thereof having at least 60% sequence identity thereto, the first peptide tag is a RumTrunk tag (SEQ ID NO: 14) or a homologue thereof having at least 60% sequence identity thereto, the second peptide tag is a MoonCake (SEQ ID NO: 115) or a homologue thereof having at least 60% sequence identity thereto, and the IRMM is the C3 binding moiety according to SEQ ID NO: 125 or a homologue thereof having at least 60% sequence identity thereto, wherein
   i) the second peptide tag is fused to the antigen, optionally by a linker; and
   ii) the first peptide tag and the IRMM are fused to the particle-forming protein, optionally by separate linkers.
36. The method according to any particle-forming one of items 1 and 3 to 26, wherein the particle-forming protein is AP205 (SEQ ID NO: 143) or a homologue thereof having at least 60% sequence identity thereto, the first peptide tag is a MoonCake (SEQ ID NO: 115) or a homologue thereof having at least 60% sequence identity thereto, and the IRMM is the CD180 agonist peptide according to SEQ ID NO: 123 or a homologue thereof having at least 60% sequence identity thereto, or the dendritic cell receptor binding peptide according to SEQ ID NO: 127 or a homologue thereof having at least 60% sequence identity thereto, wherein
   i) the first peptide tag is fused to the particle-forming protein, optionally by a linker; and
   ii) the IRMM is fused to the first peptide tag, optionally by a linker.
37. The method according to any one of items 1 and 3 to 26, wherein the particle-forming protein is AP205 (SEQ ID NO: 143) or a homologue thereof having at least 60% sequence identity thereto, the first peptide tag is a MoonCake (SEQ ID NO: 115) or a homologue thereof having at least 60% sequence identity thereto, and the IRMM is selected from the group consisting of a CD180 agonist peptide, such as the peptide according to SEQ ID NO: 123 or a homologue thereof having at least 60% sequence identity thereto, a dendritic cell receptor binding peptide, such as the peptide according to SEQ ID NO: 127 or a homologue thereof having at least 60% sequence identity thereto, a mast cell-activating peptide, such as mastoparan-7 (M7) according to SEQ ID NO: 129 or a homologue thereof having at least 60% sequence identity thereto, or SEQ ID NO: 131 or a homologue thereof having at least 60% sequence identity thereto, and a TLR2 agonist peptide, such as a peptide according to SEQ ID NO: 133 or a homologue thereof having at least 60% sequence identity thereto, wherein
   i) the first peptide tag is fused to the particle-forming protein, optionally by a linker; and
   ii) the IRMM is fused to the particle-forming protein, optionally by a linker.
38. The method according to any one of items 1 and 3 to 26, wherein the particle-forming protein is AP205 (SEQ ID NO: 143) or a homologue thereof having at least 60% sequence identity thereto, the first peptide tag is a RumTrunk D9N tag (SEQ ID NO: 13) or a homologue thereof having at least 60% sequence identity thereto, and the IRMM is selected from the group consisting of a CD180 agonist peptide, such as the peptide according to SEQ ID NO: 123 or a homologue thereof having at least 60% sequence identity thereto, a dendritic cell receptor binding peptide, such as the peptide according to SEQ ID NO: 127 or a homologue thereof having at least 60% sequence identity thereto, a mast cell-activating peptide, such as mastoparan-7 (M7) according to SEQ ID NO: 129 or a homologue thereof having at least 60% sequence identity thereto, or SEQ ID NO: 131 or a homologue thereof having at least 60% sequence identity thereto, and a TLR2 agonist peptide, such as a peptide according to SEQ ID NO: 133 or a homologue thereof having at least 60% sequence identity thereto, wherein
   i) the first peptide tag is fused to the particle-forming protein, optionally by a linker; and
   ii) the IRMM is fused to the particle-forming protein, optionally by a linker.
39. The method according to any one of the preceding items, wherein the composition comprises at least a first and a second IRMMs, wherein the first IRMM is an agonist of TLR4, such as a peptide according to SEQ ID NO: 121 or a homologue thereof having at least 60% sequence identity thereto, and the second IRMM is an agonist of CD180, such as a peptide according to SEQ ID NO: 123 or a homologue thereof having at least 60% sequence identity thereto.
40. The method according to any one of the preceding items, wherein the composition comprises at least a first and a second IRMMs, wherein the first IRMM is an agonist of TLR4, such as a peptide according to SEQ ID NO: 121 or a homologue thereof having at least 60% sequence identity thereto, and the second IRMM is an agonist of TLR2, such as a peptide according to SEQ ID NO: 133 or a homologue thereof having at least 60% sequence identity thereto.
41. The method according to any one of the preceding items, wherein administration of the composition increases or decreases an antibody immune response, such as by increasing, inhibiting or decreasing antigen-specific antibody production, compared to administering the same composition not comprising said IRMM.
42. The method according to any one of the preceding items, wherein administration of the composition increases total antigen-specific IgG production, compared to administering the same composition not comprising said IRMM.
43. The method according to any one of the preceding items, wherein administration of the composition increases or decreases a cellular immune response, such as by increasing, inhibiting or decreasing an antigen-specific cellular immune response, compared to administering the same composition not comprising said IRMM.
44. The method according to any one of the preceding items, wherein administration of the composition directs an antigen-specific immune response toward a specific IgG profile, such as an increased human IgG1 response or an increased human IgA response, compared to administering the same composition not comprising said IRMM.
45. The method according to any one of the preceding items, wherein administration of the composition leads to an altered ratio of induction of antigen-specific CD4⁺ T cells vs antigen-specific CD8⁺ T cells, such as an increased of induction of antigen-specific CD4⁺ T cells vs antigen-specific CD8⁺ T cells, or a decreased ratio of induction of antigen-specific CD4⁺ T cells vs antigen-specific CD8⁺ T cells, compared to administering the same composition not comprising said IRMM.
46. The method according to any one the preceding items, wherein the composition comprises at least two IRMMs, which synergistically modulate the immune response, such as by activation of multiple TLRs or by activation of surface receptors on multiple different types of immune cells.
47. The method according to any one of the preceding items, wherein the first, second, third and/or fourth peptide tag is derived from the splitting of a protein containing an intramolecular isopeptide bond, optionally identified from a library by using a known binding partner, or designed in silico, to obtain complementary binding partners each containing a reactive amino acid involved in isopeptide bond formation.
48. The method according to any one of the preceding items, wherein the first, second, third and/or fourth peptide tag comprises a tag selected from the group consisting of a SpyTag (SEQ ID NO: 1), a SdyTag (SEQ ID NO: 2), a SnoopTag (SEQ ID NO: 3), a PhoTag (SEQ ID NO: 4), an EntTag (SEQ ID NO: 5), a KTag, a BacTag (SEQ ID NO: 15), a Bac2Tag (SEQ ID NO: 16), a Bac3Tag (SEQ ID NO: 17), a Bac4Tag (SEQ ID NO: 18), a RumTrunk D9N tag (SEQ ID NO: 13), a RumTrunk tag SEQ ID NO: 14), a Rum7Tag (SEQ ID NO: 12), a RumTag (SEQ ID NO: 6), a Rum2Tag (SEQ ID NO: 7), a Rum3Tag (SEQ ID NO: 8), a Rum4Tag (SEQ ID NO: 9), a Rum5Tag (SEQ ID NO: 10), a Rum6Tag (SEQ ID NO: 11), a Bac5Tag (SEQ ID NO: 19), a PsCsTag (SEQ ID NO: 161), and respective homologues thereof having at least 60% sequence identity thereto.
49. The method according to any one of the preceding items, wherein the first, second, third and/or fourth peptide tag comprises a tag selected from the group consisting of a SpyCatcher (SEQ ID NO: 21) or a homologue thereof having at least 60% sequence identity thereto, a SdyCatcher (SEQ ID NO: 22) or a homologue thereof having at least 60% sequence identity thereto, a SnoopCatcher (SEQ ID NO: 23) or a homologue thereof having at least 60% sequence identity thereto, and an esther-forming split-protein pair.
50. The method according to any one of the preceding items, wherein the first, second, third and/or fourth peptide tag comprises a tag selected from the group consisting of FimP domain 3 (SEQ ID NO: 93), Streptococcal ancillary pilin Domain 2 (SEQ ID NO: 95), Streptococcal ancillary pilin Domain 3 (SEQ ID NO: 97), Major Pilin SpaD Domain 3 (SEQ ID NO: 99), Pilin subunit (SpaA) domain 2 (SEQ ID NO: 101), Pilin subunit (SpaA) domain 3 (SEQ ID NO: 103), Surface protein Spb1 domain 3 (SEQ ID NO: 105), PsCsCatcher (SEQ ID NO: 107), RgA Catcher (SEQ ID NO: 109), Major Pilin SpaD Domain 1 (SEQ ID NO: 111), QueenCatcher (SEQ ID NO: 113), MoonCake (SEQ ID NO: 115), Kat I (SEQ ID NO: 117) Clib9 (SEQ ID NO: 119), and respective homologues thereof having at least 60% sequence identity thereto.
51. The method according to any one of the preceding items, wherein the composition further comprises a SpyLigase (SEQ ID NO: 147) or a homologue thereof having at least 60% sequence identity thereto, whereby peptide tags may be locked together.
52. The method according to any one of the preceding items, wherein one of the first and second peptide tags is an SdyTag (SEQ ID NO: 2) or a homologue thereof having at least 60% sequence identity thereto and the other of the first and second peptide tags is an SdyCatcher (SEQ ID NO: 22) or a homologue thereof having at least 60% sequence identity thereto.
53. The method according to any one of the preceding items, wherein one of the first and second peptide tags is a SpyTag (SEQ ID NO: 1) or a homologue thereof having at least 60% sequence identity thereto and the other of the first and second peptide tags is a SpyCatcher (SEQ ID NO: 21) or a homologue thereof having at least 60% sequence identity thereto.
54. The method according to any one of the preceding items, wherein one of the first and second peptide tags is a SnoopTag (SEQ ID NO: 3) or a homologue thereof having at least 60% sequence identity thereto and the other of the first and second peptide tags is a SnoopCatcher (SEQ ID NO: 23) or a homologue thereof having at least 60% sequence identity thereto.
55. The method according to any one of the preceding items, wherein one of the first and second peptide tags is a RumTrunk D9N Tag (SEQ ID NO: 13) or a homologue thereof having at least 60% sequence identity thereto and the other of the first and second peptide tags is a MoonCake (SEQ ID NO: 115) or a homologue thereof having at least 60% sequence identity thereto.
56. The method according to any one of the preceding items, wherein one of the first and second peptide tags is a RumTrunk D9N Tag (SEQ ID NO: 13) or a homologue thereof having at least 60% sequence identity thereto and the other of the first and second peptide tags is a Kat I (SEQ ID NO: 117) or a homologue thereof having at least 60% sequence identity thereto.
57. The method according to any one of the preceding items, wherein one of the first and second peptide tags is a RumTag (SEQ ID NO: 6) or a homologue thereof having at least 60% sequence identity thereto and the other of the first and second peptide tags is a MoonCake (SEQ ID NO: 115) or a homologue thereof having at least 60% sequence identity thereto.
58. The method according to any one of the preceding items, wherein one of the first and second peptide tags is a RumTag (SEQ ID NO: 6) or a homologue thereof having at least 60% sequence identity thereto and the other of the first and second peptide tags is a Kat I (SEQ ID NO: 117) or a homologue thereof having at least 60% sequence identity thereto.
59. The method according to any one of the preceding items, wherein the first peptide tag, the second peptide tag, the third peptide tag and/or the fourth peptide tag is a first affinity tag, a second affinity tag, a third affinity tag, and/or a fourth affinity tag, respectively.
60. The method according to item 59, wherein one of the first and second affinity tags is a biotin and the other of the first and second affinity tags is a streptavidin, or wherein one of the third and fourth affinity tags is a biotin, such as the biotin according to SEQ ID NO: 153 or a homologue thereof having at least 60% sequence identity thereto, and the other of the third and fourth affinity tags is a streptavidin, such as the streptavidin according to SEQ ID NO: 155 or a homologue thereof having at least 60% sequence identity thereto.
61. The method according to any one of items 59 to 60, wherein one of the first and second affinity tags is a first component of a two-component nanoparticle and the other of the first and second affinity tags is a second component of said two-component nanoparticle, such as wherein one of the first and second affinity tags is 153-50A (SEQ ID NO: 157) or a homologue thereof having at least 60% sequence identity thereto and the other of the first and second affinity tags is 153-50B (SEQ ID NO: 159) or a homologue thereof having at least 60% sequence identity thereto.
62. The method according to any one of the preceding items, wherein one or more first and/or third peptide tags, or one or more first and/or second affinity tags, are fused to the N-terminal end of the particle-forming protein, to the C-terminal end of the particle-forming protein and/or inserted in-frame into the coding sequence of the particle-forming protein, optionally by a linker.
63. The method according to any one of the preceding items, wherein one or more second peptide tags, or one or more second affinity tags, are fused to the N-terminal end of the antigen, to the C-terminal end of the antigen and/or inserted in-frame into the coding sequence of the antigen, optionally by a linker.
64. The method according to any one of the preceding items, wherein said antigen and/or said IRMM does not comprise a solubility tag.
65. The method according to any one of the preceding items, wherein the composition is administered for prophylaxis and/or treatment of a disease and said disease is a cancer, a lipid disorder, a cardiovascular disease, an immune-inflammatory disease, a respiratory disease, a gastrointestinal disease, a chronic disease, a neurological disease, an infectious disease and/or an allergic reaction/disease.
66. The method according to any one of the preceding items, wherein the subject suffers from a disease selected from the group consisting of a cancer, a lipid disorder, a cardiovascular disease, an immune-inflammatory disease, a respiratory disease, a gastrointestinal disease, a chronic disease, a neurological disease, an infectious disease and an allergic reaction/disease.
67. The method according to item 65 or 66, wherein the cancer is selected from the group consisting of breast cancer, gastric cancer, ovarian cancer, glioblastoma, bone cancer, skin cancer and uterine serous carcinoma.
68. The method according to any one of items 65 to 67, wherein the lipid disorder is selected from the group consisting of a lipid disorder such as hyperlipidemia, type I, type II, type Ill, type IV, or type V hyperlipidemia, secondary hypertriglyceridemia, hypercholesterolemia, familial hypercholesterolemia, xanthomatosis and cholesterol acetyltransferase deficiency.
69. The method according to any one of items 65 to 68, wherein the cardiovascular disease is selected from the group consisting of dyslipidemia, atherosclerosis, a coronary artery disease and/or hypercholesterolemia.
70. The method according to any one of items 65 to 69, wherein the chronic or immune-inflammatory disease is selected from the group consisting of eosinophilic asthma, allergy, nasal polyposis, atopic dermatitis, eosinophilic esophagitis, hypereosinophilic syndrome, contact allergy and Churg-Strauss syndrome.
71. The method according to any one of items 65 to 70, wherein the neurological disease is Alzheimer's disease.
72. The method according to any one of items 65 to 71, wherein the infectious disease is selected from the group consisting of malaria, tuberculosis, schistosomiasis, and a disease caused by a virus, such as a coronavirus, for example SARS-CoV-2, malaria, tuberculosis, HIV, HCV, Dengue fever, Chikungunya, Yellow fever, HBV or influenza.
73. The method according to any one of the preceding items, wherein the antigen is a polypeptide, peptide and/or an antigenic fragment of a polypeptide associated with or specific for an abnormal physiological response.
74. The method according to item 73, wherein the abnormal physiological response is an autoimmune disease, an allergic reaction and/or a cancer.
75. The method according to any one of the preceding items, wherein said antigen is a protein, peptide and/or an antigenic fragment from the group consisting of cancer-specific polypeptides, polypeptides associated with cardiovascular diseases, polypeptides associated with asthma, polypeptides associated with nasal polyposis, polypeptides associated with atopic dermatitis, polypeptides associated with eosinophilic esophagitis, polypeptides associated with hypereosinophilic syndrome, polypeptides associated with Churg-Strauss syndrome and polypeptides associated with pathogenic organisms.
76. The method according to any one of the preceding items, wherein said antigen is a protein, peptide and/or an antigenic fragment of Severe acute respiratory syndrome coronavirus 2 (SARS-CoV-2).
77. The method according to any one of the preceding items, wherein the antigen is selected from the group consisting of: hemagglutinin, GD2, EGF, EGF-R, CEA, CD52, CD21, neuraminidase, human melanoma protein gp100, human melanoma protein melan-A/MART1, HIV envelope protein, M2e, VAR2CSA, ICAM1, CSP, Dengue virus NS1, Dengue virus envelope protein, Chikungunya virus envelope protein, tyrosinase, HCV E2, NA17-A nt, MAGE-3, HPV 16 E7, HPV L2, PD1, PD-L1, CTLA-4, p53, hCG, Fel d1, EGRFvlll, endoglin, ANGPTL-3, CSPG4, CTLA-4, HER2, IgE, IL-1 beta, IL-5, IL-13, IL-17, IL-22, IL-31, IL-33, TSLP, NGF, (IHNV) G-protein, a lymphotoxin such as lymphotoxin α or β, a lymphotoxin receptor, a receptor activator of nuclear factor kB ligand, vascular endothelial growth factor VEGF, a VEGF receptor, IL-23 p19, ghrelin, CCL21, CXCL12, SDF-1, M-CSF, MCP-1, endoglin, GnRH, TRH, eotaxin, bradykinin, BLC, TNF-α, amyloid β peptide A, angiotensin, gastrin, progastrin, CETP, CCR5, C5a, CXCR4, Des-Arg-bradykinin, GnRH peptide, angiotensin peptide, TNF peptide, or an antigenic fragment or antigenic variant thereof.
78. The method according to any one of the preceding items, wherein the antigen is selected from the group consisting of a SARS-CoV-2 envelope protein, a SARS-CoV-2 spike protein, such as the SARS-CoV-2 spike protein RBD (SEQ ID NO: 145) or a homologue thereof having at least 60% sequence identity thereto, a SARS-CoV-2 nucleocapsid protein and a SARS-CoV-2 envelope protein.
79. The method according to any one of the preceding items for use in treatment of an infectious disease, wherein the antigen is a protein or a peptide specific to the pathogenic organism which causes the infectious disease, or a fragment thereof.
80. The method according to any one of the preceding items, wherein the antigen is capable of eliciting an immune reaction in an animal, such as a mammal, such as a Homo sapiens, cow, pig, horse, sheep, goat, llama, mouse, rat, monkey, and/or a bird, such as a chicken and/or a fish, such as a salmon.
81. The method according to any one of the preceding items, wherein the antigen further comprises a polyhistidine tag.
82. The method according to any one of the preceding items, wherein the composition is boosted by administration in a form or body part different from the previous administration.
83. The method according to any one of the preceding items, wherein the composition is administered to the area most likely to be the receptacle of a given disease.
84. The method according to any one of the preceding items, wherein the subject is an animal, such as a mammal, such as a cow, pig, horse, sheep, goat, llama, mouse, rat, monkey, most preferably such as a human being; or a bird, such as a chicken or a fish, such as a salmon.
85. The method according to any one of the preceding items, wherein the composition is administered in combination with any other vaccine.
86. The method according to any one of the preceding items, wherein the composition forms a part of a vaccine cocktail.
87. A composition as defined in any one of the preceding items.
88. A system comprising:
   i) a particle-forming protein fused to a first peptide tag and, optionally, to a third peptide tag, optionally by a linker;
   ii) said antigen fused to a second peptide tag, optionally by a linker; and
   iii) at least one immune response modulating moiety (IRMM),
wherein
a) i), ii) and iii) are in the form of one or more polynucleotides encoding i), ii) and iii);
b) said at least one IRMM
   i. is linked to said particle-forming protein, said first peptide tag, said second peptide tag or said antigen by fusion, by an isopeptide bond, by an ester bond or by affinity conjugation, optionally by a linker; or
   ii. is fused to a fourth peptide tag, optionally by a linker; and
c) the antigen and the particle-forming protein are capable of being linked via an isopeptide bond, via an ester bond, via chemical crosslinking or via affinity conjugation between the first and the second peptide tags,
   and the at least one IRMM and the particle-forming protein are capable of being linked via an isopeptide bond, via an ester bond, via chemical crosslinking or via affinity conjugation between the third and the fourth peptide tags when present,
whereby i)-ii)-iii) form a particle displaying said antigen and said at least one IRMM.
89. A system comprising:
   i) a particle-forming protein fused to said antigen, optionally by a linker, and, optionally, to a third peptide tag, optionally by a linker; and
   ii) at least one immune response modulating moiety (IRMM)
wherein
a) i) and ii) are in the form of one or more polynucleotides encoding i) and ii);
b) said at least one IRMM
   i. is linked to said particle-forming protein or said antigen by fusion, by an isopeptide bond, by an ester bond or by affinity conjugation, optionally by a linker; or
   ii. is fused to a fourth peptide tag, optionally by a linker; and
c) the at least one IRMM and the particle-forming protein are capable of being linked via an isopeptide bond, via an ester bond, via chemical crosslinking or via affinity conjugation between the third and the fourth peptide tags when present,
whereby i)-ii) form a particle displaying said antigen and said at least one IRMM.
90. The system according to any one of items 88 to 89, wherein the first peptide tag, the second peptide tag, the third peptide tag, the fourth peptide tag, the particle-forming protein, the antigen and/or the IRMM are as defined in any one of items 1 to 81.
91. The system according to any one items 88 to 90, wherein said particle-forming protein fused to said first peptide tag and, optionally, to said third peptide tag, is encoded by a first polynucleotide.
92. The system according to any one of items 88 and 90 to 91, wherein said antigen fused to said second peptide tag is encoded by a second polynucleotide.
93. The system according to any one of items 88 to 92, wherein said at least one IRMM fused to a fourth peptide tag is encoded by a third polynucleotide.
94. The system according to any one of items 91 to 92, wherein said first and/or second polynucleotide further encodes said at least one IRMM.
95. The system according to any one of items 91 to 92, wherein the first polynucleotide, the second polynucleotide and the third polynucleotide are all DNA polynucleotides or RNA polynucleotides.
96. The system according to any one of items 91 to 95, wherein the first, second and third polynucleotides are comprised within one vector such as a viral vector or a plasmid, or wherein the first and second polynucleotides are comprised within two vectors such as two viral vectors; two plasmids; or one viral vector and one plasmid, or such as wherein the first, second and third polynucleotides are comprised within three vectors such as three viral vectors; three plasmids; or at least one viral vector and at least one plasmid.
97. The system according to item 96, wherein the viral vector is an adenoviral vector or a modified vaccinia Ankara (MVA) vector.
98. The system according to any one of items 88 to 96, wherein the system comprises or consists of a plasmid.
99. The system according to item 98, wherein said plasmid is pVAX1.
100. The system according to any one of items 88 to 99, wherein the system comprises or consists of an mRNA.
101. The system according to any one of items 91 to 100, wherein the first polynucleotide comprises or consists of a first polynucleotide encoding said particle-forming protein comprising said first peptide tag, optionally said third protein tag and optionally said at least one IRMM, the second polynucleotide comprises or consists of a second polynucleotide encoding said antigen comprising said second peptide tag and optionally said at least one IRMM, and the third polynucleotide comprises or consists of a third polynucleotide encoding said at least one IRMM fused to said fourth peptide tag.
102. The system according to any one of items 88 to 101, wherein the first polynucleotide, the second polynucleotide and, optionally, the third polynucleotides are comprised within the same nucleic acid molecule, or within two or three different nucleic acid molecules.
103. The system according to any one of items 88 to 102, further comprising a first promoter upstream of the first polynucleotide.
104. The system according to any one of items 88 to 103, further comprising a second promoter upstream of the second polynucleotide.
105. The system according to any one of items 89 to 103, further comprising a third promoter upstream of the third polynucleotide.
106. The system according to any one of items 88 to 104, wherein the first promoter is a constitutive promoter and the second promoter is an inducible promoter, such as a vitamin D-inducible promoter.
107. The system according to any one of items 88 to 104, wherein the first and the second promoters are constitutive promoters.
108. The system according to any one of items 88 to 107, wherein the first polynucleotide further comprises a secretion or excretion signal, whereby the particle-forming protein fused to the first peptide tag and, optionally, the third peptide tag, is secreted or excreted from the endoplasmic reticulum of the cell.
109. The system according to any one of items 88 to 108, wherein the second polynucleotide further comprises a secretion or excretion signal, whereby the antigen fused to the second peptide tag is secreted or excreted from the endoplasmic reticulum of the cell.
110. The system according to any one of items 88 to 109, wherein the third polynucleotide further comprises a secretion or excretion signal, whereby the IRMM fused to the fourth peptide tag is secreted or excreted from the endoplasmic reticulum of the cell.
111. The system according to any one of items 88 to 110, wherein the secretion or excretion signal comprises or consists of SEQ ID NO: 79, SEQ ID NO: 80, SEQ ID NO: 81, SEQ ID NO: 82, SEQ ID NO: 83 or SEQ ID NO: 84 or a variant thereof having at least 60% sequence identity thereto.
112. A system of cells expressing:
   i) a particle-forming protein fused to a first peptide tag and, optionally, to a third peptide tag, optionally by a linker;
   ii) said antigen fused to a second peptide tag, optionally by a linker; and
   iii) at least one immune response modulating moiety (IRMM),
wherein
a) i), ii) and iii) are in the form of one or more polypeptides or one or more polynucleotides encoding i), ii) and iii);
b) said at least one IRMM
   i. is linked to said particle-forming protein, said first peptide tag or said antigen by fusion, by an isopeptide bond or by affinity conjugation, optionally by a linker; or
   ii. is fused to a fourth peptide tag, optionally by a linker; and
c) the antigen and the particle-forming protein are capable of being linked via an isopeptide bond or via affinity conjugation between the first and the second peptide tags,
   and the at least one IRMM and the particle-forming protein are capable of being linked via an isopeptide bond or via affinity conjugation between the third and the fourth peptide tags when present,
whereby i)-ii)-iii) form a particle displaying said antigen and said at least one IRMM.
113. A system of cells expressing:
   i) a particle-forming protein fused to said antigen, optionally by a linker, and, optionally, to a third peptide tag, optionally by a linker; and
   ii) at least one immune response modulating moiety (IRMM)
      wherein
a) i) and ii) are in the form of one or more polypeptides or one or more polynucleotides encoding i) and ii);
b) said at least one IRMM
   i) is linked to said particle-forming protein or said antigen by fusion, by an isopeptide bond or by affinity conjugation, optionally by a linker; or
   ii) is fused to a fourth peptide tag, optionally by a linker; and
c) the at least one IRMM and the particle-forming protein are capable of being linked via an isopeptide bond or via affinity conjugation between the third and the fourth peptide tags when present,
whereby i)-ii) form a particle displaying said antigen and said at least one IRMM.
114. The system of cells according to any one of items 112 to 113, comprising the system according to any one of items 88 to 111.
115. A host cell, wherein the host cell comprises a system according to any one of items 88 to 111.
116. The host cell according to item 115, wherein the host cell is selected from the group consisting of a bacterial cell, a yeast cell, a fungal cell, a plant cell, a mammalian cell and an insect cell.
117. The composition according to item 87, the system according to any one of items 88 to 111, and/or the system of cells or host cell according to any one of items 112 to 116, for use in a method for inducing an immune response in a subject, the method comprising the step of administering said composition, polynucleotide or system to said subject at least once.
118. A kit of parts comprising
   i) a composition as defined in any one of the preceding items or a system according to any one of items 88 to 111, and
   ii) optionally, a medical instrument or other means for administering the composition, and
   iii) instructions for use.
119. The kit of parts according to item 118, comprising a second active ingredient.
120. A method of modulating an immune response of a subject to an antigen, the method comprising a step of administering a composition to said subject at least once, wherein the composition comprises:
   i) said antigen; and
   ii) at least one CD180 agonist,
wherein i), and ii) are provided as one or more polypeptides or as one or more polynucleotides encoding i), and ii).
121. The method according to item 120, wherein said CD180 agonist is a peptide with the sequence as set forth in SEQ ID NO: 123, or a CD180 agonist peptide with at least 80%, such as at least 85%, such as least 90%, or such as at least 95% sequence identity thereto.
122. The method according to any one of items 120 to 121, wherein the composition further comprises a particle-forming protein, wherein said particle-forming protein is provided as a polypeptide or as one or more polynucleotides encoding the particle-forming protein.

1. A method of modulating an immune response of a subject to an antigen, the method comprising a step of administering a composition to said subject at least once, wherein the composition comprises:
i) a particle-forming protein fused to a first peptide tag;
ii) said antigen, fused to a second peptide tag and
iii) at least one immune response modulating moiety (IRMM),
wherein
a) i), ii) and iii) are provided as one or more polypeptides or as one or more polynucleotides encoding i), ii) and iii);
b) said at least one IRMM
i. is linked to said particle-forming protein, said first peptide tag, said second peptide tag or said antigen by fusion or by an isopeptide bond; or
ii. is fused to a fourth peptide tag, and the particle-forming protein is fused to a third peptide, wherein the at least one IRMM and the particle-forming protein are capable of being linked via an isopeptide bond between the third and the fourth peptide tags; and
c) the antigen and the particle-forming protein are capable of being linked via an isopeptide bond between the first and the second peptide tags,
whereby i)-ii)-iii) form a particle displaying said antigen and said at least one IRMM, or
wherein the composition comprises:
i) a particle-forming protein, fused to said antigen; and
ii) at least one immune response modulating moiety (IRMM),
wherein
a) i) and ii) are provided as one or more polypeptides or as one or more polynucleotides encoding i) and ii);
b) said at least one IRMM
i. is linked to said particle-forming protein or said antigen by fusion or by an isopeptide bond; or
ii. is fused to a fourth peptide tag, and the particle-forming protein is fused to a third peptide, wherein the at least one IRMM and the particle-forming protein are capable of being linked via an isopeptide bond between the third and the fourth peptide tags;
whereby i)-ii) form a particle displaying said antigen and said at least one IRMM.

2. (canceled)

3. The method according to claim 1, wherein the IRMM is an immune cell targeting moiety, such as wherein the IRMM is capable of binding to a surface receptor of an immune cell.

4. The method according to claim 1, wherein the particle-forming protein comprises an internal IRMM.

5. The method according to claim 1, wherein the IRMM and/or the further IRMM is selected from the group consisting of a Toll-like receptor (TLR) agonist, a dendritic cell receptor binding moiety, a mast cell receptor binding moiety, a universal T cell epitope, a Complement component 3 (C3) binding moiety, an epitope recognized by a regulatory T cell, a Dectin-1 receptor targeting moiety, a cytokine or a chemokine, optionally wherein the further IRMM is the first, second or third IRMM.

6. The method according to claim 1, wherein the composition comprises at least two IRMMs, which synergistically modulate the immune response, such as by activation of multiple TLRs or by activation of surface receptors on multiple different types of immune cells.

7. The method according to claim 1, wherein said antigen and/or said IRMM does not comprise a solubility tag.

8. The method according to claim 1, wherein the composition is administered for prophylaxis and/or treatment of a disease and said disease is a cancer, a lipid disorder, a cardiovascular disease, an immune-inflammatory disease, a respiratory disease, a gastrointestinal disease, a chronic disease, a neurological disease, an infectious disease and/or an allergic reaction/disease.

9. The method according to claim 1, wherein the subject suffers from a disease selected from the group consisting of a cancer, a lipid disorder, a cardiovascular disease, an immune-inflammatory disease, a respiratory disease, a gastrointestinal disease, a chronic disease, a neurological disease, an infectious disease and an allergic reaction/disease.

10. The method according to claim 1, wherein the particle-forming protein is AP205 as set forth in SEQ ID NO: 143.

11. The method according to claim 1, wherein the particle-forming protein is fused to the first peptide tag by a linker.

12. The method according to claim 1 wherein the antigen is fused to the second peptide tag by a linker.

13. The method according to claim 1, wherein the at least one IRMM is linked to the particle-forming protein by a linker.

14. The method according to claim 1, wherein the at least one IRMM is linked to the antigen by a linker.

15. The method according to claim 1, wherein the at least one IRMM is linked to the fourth peptide by a linker.

16. The method according to claim 1, wherein the at least one IRMM is linked to the second peptide tag by a linker.

17. The method according to claim 1, wherein the third peptide tag is fused to the particle-forming protein by a linker.

18. A composition as defined in claim 1.

19. A system comprising:
i) a particle-forming protein fused to a first peptide tag;
ii) an antigen fused to a second peptide tag; and
iii) at least one immune response modulating moiety (IRMM),
wherein
a) i), ii) and iii) are in the form of one or more polynucleotides encoding i), ii) and iii);
b) said at least one IRMM
i. is linked to said particle-forming protein, said first peptide tag, said second peptide tag or said antigen by fusion or by an isopeptide bond; or
ii. is fused to a fourth peptide tag, and the particle-forming protein is fused to a third peptide, wherein the at least one IRMM and the particle-forming protein are capable of being linked via an isopeptide bond between the third and the fourth peptide tags; and
c) the antigen and the particle-forming protein are capable of being linked via an isopeptide bond between the first and the second peptide tags,
whereby i)-ii)-iii) form a particle displaying said antigen and said at least one IRMM, or
a system comprising:
i) a particle-forming protein fused to an antigen; and
ii) at least one immune response modulating moiety (IRMM)
wherein i) and ii) are provided as one or more polynucleotides encoding i) and ii); and
wherein:
a) said at least one IRMM
is linked to said particle-forming protein by fusion or by an isopeptide bond by an ester bond or by affinity conjugation;
b) said at least one IRMM is linked to an antigen by fusion or by an isopeptide bond, by an ester bond or by affinity conjugation; or
c) said particle-forming protein is fused to a third peptide tag, said at least one IRMM is fused to a fourth peptide tag, and said
at least one IRMM and the particle-forming protein are capable of being linked via an isopeptide bond, via an ester bond, via chemical crosslinking or via affinity conjugation, between the third and the fourth peptide tags,
whereby i)-ii) form a particle displaying said antigen and said at least one IRMM.

20. (canceled)

21. The system according to claim 19, wherein the first, second and third polynucleotides are comprised within one vector such as a viral vector or a plasmid, or wherein the first and second polynucleotides are comprised within two vectors such as two viral vectors; two plasmids; or one viral vector and one plasmid, or such as wherein the first, second and third polynucleotides are comprised within three vectors such as three viral vectors; three plasmids; or at least one viral vector and at least one plasmid.

22.-42. (canceled)
